(19) United States
(12) Patent Application Publication
(10) Pub. No.: US 2009/0069741 A1
(43) Pub. Date: Mar. 12, 2009

(54) Methods And Devices For Fractional Ablation Of Tissue For Substance Delivery

(75) Inventors: Gregory B. Altshuler, Lincoln, MA (US); Valery V. Tuchin, Saratov (RU); Ilya Yaroslavsky, North Andover, MA (US); James Childs, Bolton, MA (US); David Tabatadze, Worcester, MA (US); Andrei V. Erofeev, North Andover, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 12/206,426

(22) Filed: Sep. 8, 2008

Related U.S. Application Data
(63) Continuation-in-part of application No. 11/966,468, filed on Dec. 28, 2007.
Application No. 11/966,468 is a continuation-in-part of application No. 11/966,538, filed on Dec. 28, 2007.
Application No. 11/966,538 is a continuation-in-part of application No. 11/966,597, filed on Dec. 28, 2007.
Application No. 11/966,597 is a continuation-in-part of application No. 11/966,625, filed on Dec. 28, 2007.
Application No. 11/966,468 is a continuation-in-part of application No. 11/097,841, filed on Apr. 1, 2005.
Application No. 11/097,841 is a continuation-in-part of application No. 11/098,000, filed on Apr. 1, 2005.
Application No. 11/097,841 is a continuation-in-part of application No. 11/098,036, filed on Apr. 1, 2005.
Application No. 11/966,468 is a continuation-in-part of application No. 11/098,015, filed on Apr. 1, 2005.
Application No. 11/966,538 is a continuation-in-part of application No. 11/097,841, filed on Apr. 1, 2005.
Application No. 11/097,841 is a continuation-in-part of application No. 11/098,000, filed on Apr. 1, 2005.
Application No. 11/098,000 is a continuation-in-part of application No. 11/098,036, filed on Apr. 1, 2005.
Application No. 11/098,036 is a continuation-in-part of application No. 11/098,015, filed on Apr. 1, 2005.
Application No. 11/966,597 is a continuation-in-part of application No. 11/097,841, filed on Apr. 1, 2005.
Application No. 11/097,841 is a continuation-in-part of application No. 11/098,000, filed on Apr. 1, 2005.
Application No. 11/098,000 is a continuation-in-part of application No. 11/098,036, filed on Apr. 1, 2005.
Application No. 11/098,036 is a continuation-in-part of application No. 11/098,015, filed on Apr. 1, 2005.
Application No. 11/966,625 is a continuation-in-part of application No. 11/097,841, filed on Apr. 1, 2005.
Application No. 11/097,841 is a continuation-in-part of application No. 11/098,000, filed on Apr. 1, 2005.
Application No. 11/098,000 is a continuation-in-part of application No. 11/098,036, filed on Apr. 1, 2005.
Application No. 11/098,036 is a continuation-in-part of application No. 11/098,015, filed on Apr. 1, 2005.
Application No. 11/097,841 is a continuation-in-part of application No. 60/561,052, filed on Apr. 9, 2004.
Application No. 11/098,000 is a continuation-in-part of application No. 60/641,616, filed on Jan. 5, 2005.
Application No. 11/098,036 is a continuation-in-part of application No. 60/641,616, filed on Jan. 5, 2005.
Application No. 11/098,015 is a continuation-in-part of application No. 60/641,616, filed on Jan. 5, 2005.
(60) Provisional application No. 60/970,420, filed on Sep. 6, 2007.
(60) Provisional application No. 60/877,826, filed on Dec. 29, 2006.
(60) Provisional application No. 60/561,052, filed on Apr. 9, 2004.
(60) Provisional application No. 60/561,052, filed on Apr. 9, 2004.
(60) Provisional application No. 60/561,052, filed on Apr. 9, 2004.
(60) Provisional application No. 60/614,382, filed on Sep. 29, 2004.
(60) Provisional application No. 60/614,382, filed on Sep. 29, 2004.
(60) Provisional application No. 60/614,382, filed on Sep. 29, 2004.
(60) Provisional application No. 60/614,382, filed on Sep. 29, 2004.
(60) Provisional application No. 60/641,616, filed on Jan. 5, 2005.
(60) Provisional application No. 60/620,374, filed on Oct. 20, 2004.
(60) Provisional application No. 60/620,734, filed on Oct. 21, 2004.
(60) Provisional application No. 60/620,734, filed on Oct. 21, 2004.
(60) Provisional application No. 60/620,734, filed on Oct. 21, 2004.

(51) Int. Cl. A61M 37/00; A61B 18/18

(52) U.S. Cl. 604/22; 606/33

(57) ABSTRACT

Methods and devices for ablating portions of a tissue volume with electromagnetic radiation (EMR) to produce lattices of EMR-treated ablation islets in the tissue are disclosed, including lattices of micro-holes, micro-grooves, and other structures. Also, methods and devices for using the ablated islets are disclosed, including to deliver chromophores, filler, drugs and other substances to the tissue volume.

### RELATED APPLICATIONS

This application claims the benefit of U.S. Provisional Application No. 60/970,420 filed Sep. 6, 2007. This application is a continuation-in-part of U.S. application Ser. Nos. 11/966,468, 11/966,538, 11/966,597, and 11/966,625 each of which was filed on Dec. 28, 2007 and which claim priority to U.S. Provisional Application No. 60/877,826, filed Dec. 29, 2006, and each of which is a continuation-in-part application of U.S. application Ser. Nos. 11/097,841, 11/098,000, 11/098,036, and 11/098,015, each of which was filed Apr. 1, 2005 and entitled “Methods and products for producing lattices of EMR-treated islets in tissues, and uses therefore” and each of which claims priority to U.S. Provisional Application No. 60/561,052, filed Apr. 9, 2004, U.S. Provisional Application No. 60/614,382, filed Sep. 29, 2004, U.S. Provisional Application No. 60/641,616, filed Jan. 5, 2005, and U.S. Provisional Application No. 60/620,734, filed Oct. 21, 2004.

Each of the applications and provisional applications identified above is incorporated herein by reference in its entirety.

### BACKGROUND OF THE INVENTION

1. Field of the Invention

The devices and methods disclosed herein relate to the ablation of soft and hard tissues with electromagnetic energy generally, including, without limitation, optical energy having wavelengths in the ultraviolet, visible and infrared ranges. Some embodiments relate to devices and methods that are used to ablate micro-holes in the treated tissue.

2. Description of the Related Art

Electromagnetic radiation, particularly in the form of laser light, has been used in a variety of cosmetic and medical applications, including uses in dermatology, dentistry, ophthalmology, gynecology, otorhinolaryngology and internal medicine. For most dermatological applications, the EMR treatment can be performed with a device that delivers the EMR to the surface of the targeted tissues. For applications in internal medicine, the EMR treatment is typically performed with a device that works in combination with an endoscope or catheter to deliver the EMR to internal surfaces and tissues.

As a general matter, existing EMR treatments are typically designed to (a) deliver one or more particular wavelengths (or a range (or ranges) of wavelengths) of EMR to a tissue to induce a particular chemical reaction, (b) deliver EMR energy to a tissue to cause an increase in temperature, or (c) deliver EMR energy to a tissue to damage or destroy cellular or extra cellular structures, such as for skin remodeling.

For skin remodeling, absorption of optical energy by water is widely used in two approaches: ablative skin resurfacing, typically performed with either CO2 (10.6 μm) or Er:YAG (2.94 μm) lasers, and non-ablative skin remodeling using a combination of deep skin heating with light from Nd:YAG (1.34 μm), Er:glass (1.56 μm) or diode laser (1.44 μm) and skin surface cooling for selective damage of sub-epidermal tissue. Non-ablative techniques offer considerably reduced risk of side effects and are much less demanding on post-operative care. However, clinical efficacy of the non-ablative procedure has not been satisfactory.

In the cosmetic field for the treatment of various skin conditions, alternative methods and devices have been developed that irradiate or cause damage in a portion of the tissue area and/or volume being treated. These methods and devices have become known as fractional technology. Fractional technology is thought to be a safer method of treatment of skin for cosmetic purposes, because tissue damage occurs within smaller sub-volumes or islets within the larger volume of tissue being treated. The tissue surrounding the islets is spared from the damage. Because the resulting islets are surrounded by neighboring healthy tissue the healing process is thorough and fast. Furthermore, it is believed that the surrounding healthy tissue aids in healing and the treatment effects of the damaged tissue.

Examples of devices that have been used to treat the skin using non-ablative procedures such as skin resurfacing include the Palomar® 1540 Fractional Handpiece, the Reliant Fraxel® SR Laser and similar devices by ActiveFX, Alma Lasers, Iridex, and Reliant Technologies.

### SUMMARY OF THE INVENTION

The present invention uses ablative fractional methods and devices to perform cosmetic and other treatments and functions on hard and soft tissue, including skin tissue. In various embodiments, examples of which are described in greater detail below, improved devices and systems for ablating tissue by producing lattices of EMR-treated islets in tissues are provided as well as improved cosmetic and medical applications of such devices and systems. For example, in one embodiment, methods and devices are described for creating lattices of ablation islets. In some embodiments, methods and devices are described for selectively damaging a portion of a tissue volume being treated by applying EMR radiation to produce a lattice of EMR-treated islets, which absorb an amount of EMR sufficient to damage the tissue by killing cells at the surface of the tissue or otherwise causing ablation of the tissue in the EMR-treated islets, but not sufficient to cause bulk tissue damage.

Other embodiments include devices and methods that allow EMR to be precisely delivered such that uniform micro-holes and other types of EMR-treated islets having very small dimensions can be reliably formed. Methods and devices are described for ablating tissue to form micro-holes, micro-grooves, micro-voids and other micro-structures. For example, methods and devices are described for creating ablation islets that are small and precisely formed, for example, micro-holes in some embodiments having diameters of approximately 1-50 μm and micro-holes in other embodiments having diameters of a magnitude that is 10% or less of the wavelength used to create the micro-holes.

Other embodiments include various uses for ablated structures, including holes, grooves, voids, and various micro-structures. In some embodiments, ablative fractional treatments of tissue provide an alternative to non-ablative techniques that produces superior results. In other embodiments, ablative fractional methods and devices can be used to ablate holes, grooves, voids and other structures into tissue for various purposes, including, without limitation, skin tightening, wrinkle reduction, application of fillers, application of biologically inert materials, application of drugs, application of chromophores, application of optically transmissive substances, application of other substances to alter the optical characteristics of the tissue, application of drugs, and the application of other substances.

As examples, some of the embodiments described provide for one or more of the following:
1. The ability to perforate and/or form holes in tissue, such as, for example, by forming holes in the skin through which a substance can be passed;
2. The ability to form EMR-treated islets that are far smaller than can be created by previous fractional treatments, such as, for example, by forming islets of treated tissue, damaged tissue, perforated tissue, tissue with holes and/or similar structures in tissues that are on the order of approximately 1 μm or less in diameter, and that have a very small pitch, for example, pitches on the order of approximately 330 μm, 220 μm, 110 μm, 10 μm, or even less for correspondingly small micro-holes;
3. The ability to perform skin rejuvenation using pure light, other EMR, other types of energy or combinations of energy and that ability to perform other procedures, such as, for example, photobiomodulation, photodynamic therapies, and other forms of therapy;
4. The ability to inject materials into tissue, such as, for example, drugs and biologically inert materials, including, without limitation, collagen, fat, cosmetics, substances capable of providing permanent protection from ultraviolet (“UV”) radiation, and tattoos;
5. The ability to deliver EMR in a highly uniform manner, such as, for example, across a curved, leveled or flattened tissue surface;
6. The ability to control the dimensions of the islets of EMR-treated tissue, such as, for example, by tuning and/or adjusting the wavelength of EMR that is applied to the tissue to modulate the dimensions of the EMR-treated islets; and
7. The ability to form many different patterns of EMR-treated tissue, including, without limitation, very small islets of EMR-treated tissue, very small islets of non-EMR-treated tissue that are surrounded by EMR-treated tissue, and larger islets of non-EMR-treated tissue surrounded by very small bands or portions of EMR-treated tissue.

In one aspect, the invention discloses a method of trans-dermal delivery of substances comprising ablating a portion of the tissue with electromagnetic radiation to form a plurality of channels extending to a depth below a surface of the tissue; delivering a substance into one or more of the channels; and sealing the one or more channels, so as to embed the substance within the channels.

In some embodiments, the substance can be delivered using one of the delivery techniques selected from the group consisting of diffusion, vesicle/particle transporters, physical mechanisms, chemical mechanisms, electrical mechanisms, electroporation, iontoporation, sonophoresis, magnetophoresis, photomechanical waves, niosomes, and transfersomes. Sealing of the one or more channels can comprise applying a healing substance to the portion of tissue and/or covering the portion of tissue, such as by applying tape or strips, or applying sutures to the portion. In some embodiments, the substance can be a drug.

At least some of the substance can be delivered to a layer of fat tissue. The substance can be heated to a temperature sufficient to denature fat tissue in the layer. The substance can be encapsulated. The substance can be allowed to diffuse into the tissue prior to treatment.

The method can also include heating and/or cooling a volume of the tissue. For example, the tissue can be cooled to a temperature less than or equal to approximately 30 degrees Celsius, or more preferably to a temperature less than or equal to approximately 5 degrees Celsius.

The channels can extend to a depth in the range of approximately 5 micrometers to 5 millimeters. The channels can extend from the surface of the skin tissue to the epidermis, the dermis or below the dermis. The channels can have a width of approximately 1 micrometer to 1 millimeters, and more preferably between approximately 50 micrometers to 100 micrometers.

The fill-factor of the channels in a cross-sectional area can be between approximately 1 and 90 percent, or more preferably between approximately 25 and 40 percent. The center-to-center distance between adjacent channels can be between approximately 10 micrometers and 5 millimeters.

The tissue can be ablated with electromagnetic radiation having one or more wavelengths of between approximately 350 and 2000 nanometer, or more preferably between approximately 190 to 350 nanometers and/or 1.3 and 12 micrometers. The electromagnetic radiation applied to the chromophore can have one or more wavelengths in the visible spectrum and/or the infrared spectrum.

The method can further comprise ablating a second portion of the tissue with electromagnetic radiation to form at least one additional channel extending a distance from a surface of the tissue. The additional channels can have dimensions different than those of the first channels. The additional channels can have different dimensions that the first channels, such as different width, shape or depth.

Another embodiment is a method for treating a volume of tissue comprising: forming a channel in the tissue by applying a first dose of electromagnetic radiation having at least one wavelength component suitable for ablating the tissue; delivering a chromophore into the channel; and heating the chromophore with a second dose of electromagnetic radiation.

Preferred variations of this embodiment can include one or more of the following. The method can further include applying a second dose of energy to the tissue. The second dose can be applied to the entire surface of the volume of tissue. The second dose can be applied to portions of the volume of tissue to produce an array of treatment islets. The substance can be delivered into the channel or can be delivered into an through the channel. Some or all of the substance can remain in the channel prior to irradiation of the chromophore, depending on the application. Similarly, some or all of the substance can be forced into or diffuse into the tissue, depending on the application.

Another embodiment is a method for performing a cosmetic procedure comprising: creating a set of voids in skin tissue by applying electromagnetic radiation; introducing a substance into the voids; and applying energy to the substance to increase the temperature of the substance.

Preferred variations of this embodiment can include one or more of the following.

The substance can be introduced into the voids by many different mechanisms, including, without limitation, by forcing at least a portion of the substance through the voids and into the skin tissue, vibrating the tissue, applying positive pressure to the tissue, alternately applying positive and negative pressure to the tissue, applying an electrical field to the tissue, applying a magnetic field to the tissue, manually manipulating the tissue, massaging the tissue, stretching the tissue, compressing the tissue, alternately stretching and compressing the tissue, applying a pressure wave to the surface of the tissue, applying a jet containing the substance to the surface of the tissue, applying energy to the substance to cause cavitation within the tissue and applying ultrasound to the surface of the tissue.

The substance can be many different forms, including, without limitation, one or more of the following: elements, compounds, compositions, mixtures, suspensions, particulates, crystals, liquids, solids, and gasses.

Another embodiment is a device for treating soft tissue. The device includes a source of optical radiation configured to generate sufficient optical radiation to ablate tissue; an output aperture configured to emit the optical radiation onto the soft tissue in a pattern of treatment areas separated by untreated areas such that the optical radiation forms voids in the tissue at the treatment areas; an optical path extending from the source of the optical radiation to the output aperture, and configured to deliver the optical radiation to the soft tissue; a chromophore applicator configured to inject a chromophore into the voids formed by the optical radiation generated by the source during operation.

Preferred variations of this embodiment can include a jet configured to spray the chromophore into the voids.

### BRIEF DESCRIPTION OF THE DRAWINGS

The following drawings are illustrative and are not meant to limit the scope of the invention as encompassed by the claims.

FIG. 1 is a schematic view of various embodiments of micro-holes.

FIG. 2 is a schematic diagram showing EMR of a beam focused to a focal point.

FIG. 3 is a graphical representation of the distribution of power density as a function of the distance along a diameter of a focal point.

FIGS. 4A and 4B are semi-schematic perspective and side views respectively of a section of a patient's skin and of equipment positioned thereon for practicing one embodiment.

FIGS. 5A and 5B are top views of various matrix arrays of cylindrical lenses, some of which are suitable for providing a line focus for a plurality of targets.

FIGS. 6A and 6B are perspective views of sets of ablated islets in the form of micro-grooves.

FIGS. 6C-6G are alternative embodiments of micro-grooves.

FIG. 7 is a side perspective diagram of a device for forming micro-holes.

FIG. 8 is a front perspective diagram of the device of FIG. 7.

FIG. 9 is a schematic diagram of an optical system of the device of FIG. 7.

FIG. 10 is a schematic diagram of a pattern of beams created by the device of FIG. 7.

FIG. 11A is a schematic diagram of a beam focused on a tissue surface.

FIG. 11B is a schematic diagram of a beam focused on a tissue surface and through an aperture to flatten the surface.

FIG. 11C is a schematic diagram of an optical system that focuses an array of beams along a curved focal plane.

FIG. 12 is a photograph of a section of paper treated using the device of FIG. 7.

FIG. 13 is a schematic diagram of an alternate optical system for generating beams.

FIG. 14 is a graphical representation of the intensity of a set of beams as a function of distance.

FIG. 15 is a graphical representation of the intensity of a second set of beams as a function of distance.

FIG. 16 is a schematic diagram of alternate profiles for a beam used to generate a set of beams.

FIG. 17 is a schematic diagram of two exemplary embodiments of micro-holes.

FIG. 18 is a graphical representation of the coefficient of absorption of EMR in human skin tissue as a function of wavelength.

FIG. 19 is a side schematic view of components of an optical system that can be used in some embodiments.

FIG. 20 is a perspective view of another embodiment.

FIG. 21 is a perspective view of yet another embodiment.

FIG. 22 is a perspective view of another embodiment for creating treatment islets.

FIG. 23 is a bottom view of another embodiment, which uses one or more capacitive imaging arrays.

FIG. 24A is a side view of an embodiment using a diode laser bar.

FIG. 24B is a perspective view of a diode laser bar that can be used in the embodiment of FIG. 24A.

FIG. 24C is a side view of yet another embodiment, which uses multiple diode laser bars.

FIG. 24D is a side view of yet another embodiment, which uses multiple diode laser bars.

FIG. 24E is a side view of yet another embodiment, which uses multiple optical fibers to deliver EMR to the tissue.

FIG. 25 is a side view of another embodiment, which uses a motor capable of moving a diode laser bar within a hand piece.

FIG. 26 is a top view of one embodiment of a diode laser bar.

FIG. 27 is a side cross-sectional view of the diode laser bar of FIG. 26.

FIG. 28A is a side view of another embodiment, which uses a fiber bundle.

FIG. 28B is a side view of another embodiment, which uses a phase mask.

FIG. 28C is a side view of another embodiment, which uses multiple laser rods.

FIG. 29 is a graphical representation estimating based on experimental data the time for a micro-hole to close as a function of the diameter of the micro-hole.

FIG. 30 is a cross-sectional view of a section of skin tissue with micro-grooves having walls that are partially compressed together.

FIG. 31 is a cross-sectional view of the section of skin tissue of FIG. 30 with the micro-grooves having walls that are fully compressed together and fixed in place.

FIG. 32 is a cross-sectional view of a section of skin tissue having a set of micro-holes used to deliver a chromophore to subcutaneous fat.

FIG. 33 is a cross-sectional view of a section of skin tissue having a set of micro-holes used to deliver a filler to a dermal layer to obscure a tattoo.

FIG. 34 is a cross-sectional view of a section of skin tissue having a set of micro-holes used to deliver a filler to a dermal layer to serve as a sunscreen.

FIG. 35 is a cross-sectional view of a section of skin tissue having a set of micro-holes used to deliver a filler to an epidermal layer to serve as a sunscreen.

FIGS. 36A and 36B are cross-sectional views of a section of skin tissue illustrating the incorporation of a filler into a micro-groove.

FIGS. 37-46 are photographs taken of experimental results using devices similar to the device of FIG. 7.

FIG. 47 is a graphical representation of the depth of penetration of a micro-hole as a function of fluence per beam.

FIGS. 48-51 are photographs taken of experimental results using devices similar to the device of FIG. 7.

FIG. 52 is a graphical representation of the depth of penetration of a micro-hole as a function of fluence per beam and showing two curves for two different configurations of a device similar to the device of FIG. 7.

FIGS. 53-60 are photographs taken of experimental results using devices similar to the device of FIG. 7.

FIG. 61 is a graphical representation of the thickness or depth of a zone of coagulation around an ablated islet as a function of pulsewidth using a laser having a wavelength of 2780 nm.

FIG. 62 is a schematic representation of a skin model having ablation islets extending from a skin surface through the stratum corneum, the epidermis and the dermis of the skin tissue.

FIG. 63 is a schematic of a diffusion model of the agent A in a layer B.

FIG. 64 is a graph showing the distribution of glycerol concentration via sample depth.

FIG. 65 is a graph showing the dependence of glycerol concentration on time for different depths.

FIG. 66 is a graph showing the experimental temporal dependence of the transmitted intensity of ex vivo pig skin sample at 633 nm treated using technology of the lattice of limited thermal damage islets in epidermis only; experiments were carried out for a 40%-glycerol solution as a chemical agent applied after SC damaging; these data were used for reconstruction of glycerol spatial-temporal distributions in the skin.

FIG. 67 is a graph showing the total permeability of different skin models calculated using Eq. (11).

FIG. 68 is a graph showing the total permeability of different skin models calculated using approximate Eq. (15).

### DETAILED DESCRIPTION

When using electromagnetic radiation (EMR) and other forms of energy to treat tissues, there are substantial advantages to producing lattices of treated islets in the tissue rather than large, continuous regions of treated tissue. The lattices are periodic patterns of islets in one, two or three dimensions in which the islets correspond to local maxima of treated tissue. The islets are separated from each other by non-treated tissue (or differently- or less-treated tissue).

The EMR-treatment results in a lattice of EMR-treated islets which have been exposed to a particular wavelength or spectrum of EMR, and which is referred to herein as a lattice of “islets.” When the absorption of EMR energy results in significant temperature elevation in the EMR-treated islets, the lattice is referred to herein as a lattice of “thermal islets.” When an amount of energy is absorbed that is sufficient to significantly disrupt cellular or intercellular structures, the lattice is referred to herein as a lattice of “damage islets.” When an amount or energy is absorbed that is sufficient to ablate the tissue being treated, the lattice is referred to herein as a lattice of “ablated islets” or “ablation islets.” An extensive discussion of the various types of EMR-treated islets (such as damage, thermal and photochemical islets) as well as the parameters and specification of devices used for form such types of islets can be found in the applications incorporated by reference above, and the bulk of that disclosure is not repeated herein

The inventors have further discovered that when ablation islets are created on a small scale, the islets have many advantages, which are described below in conjunction with various embodiments. The inventors have also discovered devices and methods for creating such islets on a small scale, referred to herein as micro-islets. Although various forms of energy can be used, including ultrasound energy, the exemplary embodiments below are chiefly described with reference to using EMR to create EMR-treated islets.

### I. Creation of Islets by Ablating Tissue

A. Ablation Islets

One specific type of EMR-treated islet that is particularly useful is the ablation islet. An ablation islet in its simplest form is a void in tissue formed by ablation processes that remove a portion of the tissue for form the void. However, due to the complexity of EMR-tissue interactions and the dynamic nature of living tissue, the islet may be more complex. The damage to the tissue in the islet is to the degree that the tissue is vacated to form empty space or is altered in composition, such as, for example, in the case of a channel of tissue that is damaged such that the channel is vacated or primarily filled with water, other fluid and/or remnants or vestiges of the damaged tissue (e.g., tissue fibers or other substances). For example, during ablation, some or all of the tissue may not be removed from the “void” and may remain in the void as desiccated tissue and/or debris from ablation processes. Furthermore, the “void” may be filled with water or other substances as the tissue reacts to the ablative injury to the tissue. Similarly, the shape of the “void” may change. For example, the walls of the “void” may partially or completely collapse as a result of tissue that is removed or as a result of the healing process. Other processes may also be involved, such as cavitation within the tissue, that will result in an alteration in the size and shape of the void. Thus, a “void” resulting from an ablative process may not necessarily result in empty space or a particular shape being formed within the tissue.

B. Micro-Islets

When the ablated islets are sufficiently small, for example, on the order of approximately 2 mm or less, the islets are also referred to herein as a lattice of “micro-islets.” In some embodiments, an ablation islet is a small volume in the tissue in which the tissue has been damaged, ablated or otherwise treated to form small holes, channels, grooves, openings, chambers and/or similar structures in the tissue. (For convenience, such structures are referred to collectively as micro-islets, micro-voids and/or micro-structures. The term micro-hole is used extensively throughout the specification as an exemplary embodiment of a micro-islet, but many other embodiments are possible. For example, the shape of the micro-islets may have many forms, including, without limitation, micro-holes, micro-grooves, micro-voids and other micro-structures. While the term “micro” connotes that the resulting structure is significantly smaller in volume than the overall volume of the tissue being treated (or, similarly, that the area of tissue to which the energy is applied to form a micro-islet is significantly smaller than the total area of tissue being treated), it does not require that the resulting micro-islet be microscopic in size. Micro-holes can be various sizes, including, without limitation, micro-holes that are macroscopic or microscopic in size. For example, a lattice of micro-holes on nail tissue can have a diameter of 50 micrometers, but much smaller micro-holes are possible, and larger micro-holes are also possible. Additionally, the orientation of the islets can be varied from normal to a tissue surface to parallel with the surface or other angles or orientations, including islets that are curved or otherwise are not formed along a straight path.

Micro-islets can be used for a variety of purposes such as, for example, the application of drugs and medicines, the injection of fillers and other inert substances, and the removal of fat tissue or other substances, skin resurfacing, skin rejuvenation, skin tightening and wrinkle removal. Micro-holes can be used as channels for the local delivery of the desirable therapeutic compound(s) to the target (treated) anatomical areas by diffusion or by employing but not limited to the other approaches, such as vesicle/particle transporters, by physical, chemical or electrical manipulations (for instance electroporation, iontoporation, sonophoresis, magnetophoresis, photomechanical waves, niosomes, transfersomes etc.). Micro-holes can be created in any tissue, such as skin, nail, bone, muscle, etc., and at any anatomical location.

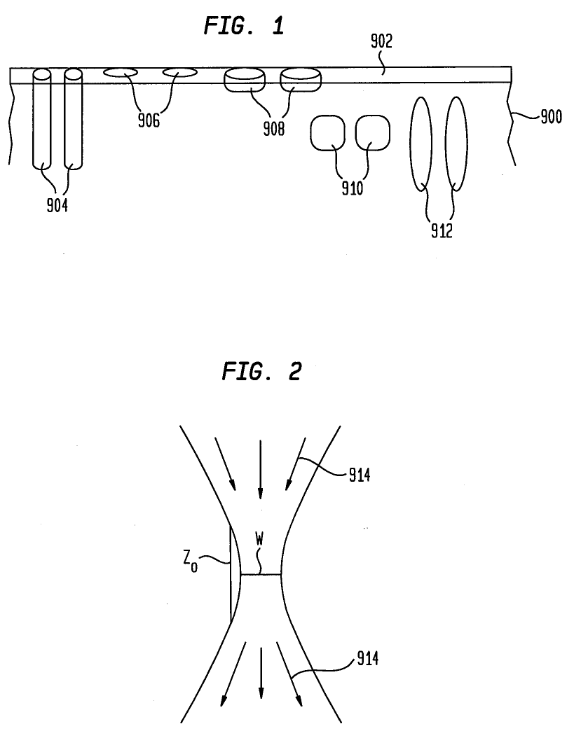

Referring to FIG. 1, examples of various micro-islet structures are shown. Micro-holes 904 are columns extending from a surface 902 of tissue 900 and into the tissue 900. Micro-holes 906 are openings that lie at the surface 902 of the tissue, but that do not extend deeply into the tissue. Micro-holes 908 are depressions that lie at the surface 902, but that extend slightly into the tissue 900 to a greater depth than micro-holes 906. Micro-holes 910 are chambers within the tissue 900 and below the surface 902. Similarly, micro-holes 912 are chambers that are elongated to for columns but that do not have an opening through the surface. The micro-holes shown in FIG. 1 are simplified for purposes of the above description. Depending on how a micro-hole is formed, its structure may be more complex. For example, a micro-hole formed by ablating tissue may have a zone or halo of damage surrounding the vacated hole. This is shown more clearly in conjunction with FIG. 17 discussed below.

In an ablative process in which micro-holes or other micro-structures are formed, treatment parameters can be chosen such that a relatively small volume or zone of coagulated tissue surrounds the volume of ablated tissue that results in a void that forms a micro-hole or other micro-structure. In other words, the ratio of the coagulated tissue volume to the ablated tissue volume can be controlled. The ratio can be very small (such as from about 10% to essentially zero), e.g., by choosing wavelengths that are highly absorbed, using short pulses of EMR, and/or quickly evacuating any ablated tissue such that heat from the tissue is not allowed to disperse to surrounding tissues. Conversely, the ratio can be made to be much larger, i.e., a relatively large volume of coagulated tissue surrounding the ablated tissue volume (such as 50% or greater), by choosing treatment parameters that allow heat to disperse into the surrounding tissue during ablation. For example, ablating tissue using wavelengths that are typically used in non-ablative processes, such as approximately 1320 nm, 1450 nm and 1540 nm, at intensities that will ablate tissue, typically would result in larger coagulation zones surrounding the volume of ablated tissue.

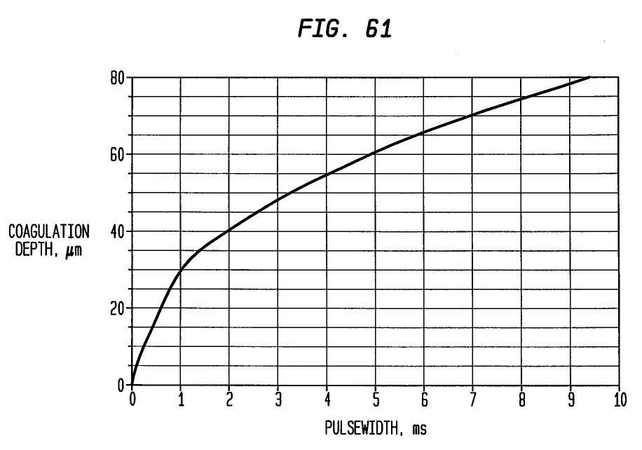

A typical zone of coagulation surrounding and/or adjacent to an ablation zone will have a thickness of approximately 5 μm to 100 μm, but other dimensions are possible. Referring to FIG. 61, the thickness of the layer of coagulated tissue surrounding the ablation void is shown as a function of pulsewidth, using the parameters listed in Table B for a laser operating at 2780 nm.

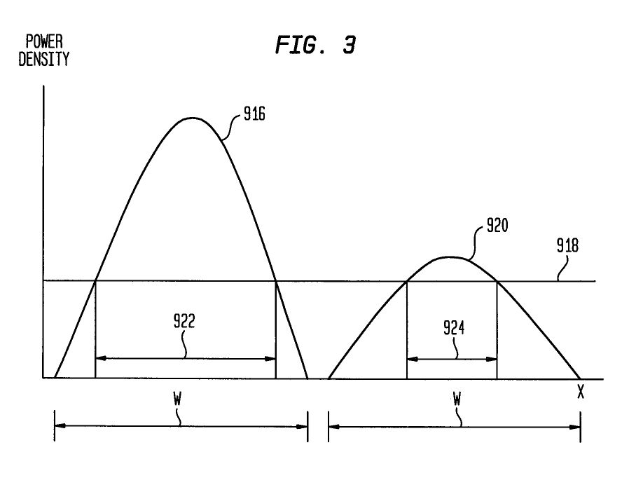

Referring to FIGS. 2 and 3, the size of a micro-hole is determined essentially by the spot size at which EMR is applied to the tissue, the power density of the EMR that is applied, the wavelength of EMR that is applied and the threshold of ablation in the tissue that is irradiated (or other thresholds, for example, the threshold of thermal damage in other embodiments). To maximize the intensity of the radiation, the spot size of a micro-hole is preferably the diameter of the focal point. Using currently available optics, therefore, micro-holes can be formed having a diameter of approximately 0.1×λ (i.e., 10% of the wavelength of the applied radiation). However, even smaller diameters are theoretically possible, depending on the quality of optics and the design of optics that are used.

The spot size that can be created (and, thus, the resulting micro-hole) is proportional to the wavelength: the smaller the wavelength, the smaller the micro-hole that can be created. FIG. 2 shows a focused beam of rays 914 of EMR in which the focal point has a diameter W greater than the wavelength of the EMR. Theoretically, the smallest spot size that is possible for an individual EMR beam is the smallest focal point that can be achieved. The smallest focal point that may be achieved has a diameter (W) that is approximately the wavelength (λ) of the EMR that is applied. (Although the term focal point is used, one skilled in the art will understand that light does not focus to a point and instead has an area with a diameter that is typically referred to as the waist of the beam.)

If non-coherent light is applied, the smallest spot size that is theoretically possible is the largest wavelength among the wavelengths that are applied to achieve a treatment effect on the tissue, such as an ablated micro-hole. This would not include longer wavelengths that do not ablate the tissue or otherwise have an effect that forms an EMR-treatment islet. For example, if one or more spectral bands of EMR are applied to the tissue, but only a subset, subsets, or sub-band(s) of the EMR are actually used to ablate or otherwise treat and form the islet, the smallest possible diameter of the resulting micro-hole will be the size of the largest wavelength in the sub-band(s) or subset(s) of EMR.

Because smaller focal areas are possible using shorter wavelengths, one effective means for creating very small micro-holes or other micro-islets is the use of an Eximer laser or another laser to produce EMR in the ultraviolet range.

The focal depth (Z₀) of the spot size is a function of the diameter of the focal point, which is determined by the following equation:

$${Z}_{0} = \frac{π * {W}^{2}}{λ} \qquad ( 1 )$$

Thus, in an example where the focal point has a diameter of 30 μm and the wavelength is 3 μm, the focal depth is approximately 943 μm.

FIG. 3 shows the power density of EMR as a function of distance across the focal point of the applied EMR. In the case shown, the EMR has been focused to an area having a diameter equal to the wavelength of the applied EMR. When EMR is applied to tissue, the power of the EMR has a roughly Gaussian distribution with the highest intensity at the mid-point of the focal point, in this case, the midpoint of the wavelength of EMR used. When the power of the applied EMR exceeds the threshold of ablation, a micro-hole is formed. (Although many embodiments include optical systems and/or elements to focus EMR at a focal point, such focusing is not required to practice many other embodiments.)

As seen in FIG. 3, the size of the holes 922 and 924 can be controlled by adjusting the power applied. In the example illustrated, a power distribution 916 exceeds a threshold of ablation 918 for approximately one-half of the focal point, i.e., the wavelength λ. However, when the power of the applied EMR is reduced, a power distribution 920 exceeds the threshold of ablation 918 over a much smaller portion of the focal point: in the case shown, approximately 0.1× the diameter of the focal point or approximately 0.1×λ. Theoretically, the micro-holes could be any non-zero number, but practically other factors may provide a lower limit to the size of the diameter of the micro-holes, such as the quality of the optics and other factors.

In other embodiments, the power density may be modulated during the formation of a single micro-hole. For example, a first pulse of EMR can be applied at a first power density and a second pulse can be applied at a different power density. If the power densities of multiple pulses are alternated in this fashion, micro-holes having varying diameters can be formed. Such micro-holes may have various benefits, for example, an increase in surface area that can be used to deliver substances such as drugs or clearing substances more effectively or at a faster rate. Similarly, the power density can be modulated, for example, between pulses, during pulses or during the application of EMR in a continuous or quasi-continuous wave, to form micro-holes of varying shapes, such as, for example a conical-like shape. A conical shape in which the narrow portion of the cone is at the surface of the tissue and in which the wider base of the cone lies within the tissue could be used to create a micro-hole having a relatively larger volume, which can be used, for example, to hold a substance, and also having a relatively small opening, which will close more quickly than a larger hole. (The closure rates of micro-holes are discussed in greater detail in conjunction with FIG. 29.)

When using ablation to form a micro-hole, the ablation is preferably performed in conjunction with a device to remove the ablated material, although this is not required. When tissue is ablated, remnants of the tissue generally remain in the micro-holes. This can increase the amount of refraction and otherwise decrease optimum performance of the device forming the micro-holes. The micro-holes are formed more precisely when the ablated material is removed. There are many embodiments possible of a system, device or method to remove tissue, such as, for example, a device that is synchronized to produce a short pulse of air at high pressure, which expels the ablated material immediately after a pulse of EMR is applied before the material has a chance to settle in the micro-hole that is being formed.

Many different embodiments are possible for removing tissue. For example, devices in which the EMR is delivered through an optical element such as a lens that is not in contact with the tissue can include a device that directs air or other gas into the space between the tissue and the optical element to remove the remnants of the ablated tissue. In embodiments where an optical element from which EMR is delivered is in contact with the tissue, other structures can be used. For example, the optical element may contain ribs, ridges, channels or other structures through which a high-pressure gas may be pulsed such that the remnants of ablated tissue are removed through those structures as the device is moved relative to the tissue during operation. Similarly, in still other embodiments, some or all of the remnants of the ablated tissue can be left within the micro-holes. However, if tissue is ablated and not subsequently vacated from the EMR-islet, additional factors will affect the characteristics of the resulting micro-hole. For example, scattering within the tissue, including the remnants of the ablated tissue, may increase and impact the size, shape and other characteristics of the micro-hole.

While the above has been discussed in terms of the threshold of ablation, the concept can be applied similarly to other types of EMR-Islets, for example, by using thresholds of damage instead of thresholds of ablation. Non-ablative techniques may be used to form similar micro-structures, such as zones of thermally damaged tissue or small zones of healthy tissue surrounded by zones of EMR-treated tissue, such as, for example, thermally treated tissue and/or ablated tissue.

C. The Shape of Ablated EMR-Islets

The optical islets can be formed essentially in any shape, limited only by the ability to control EMR beams within the tissue. Thus, depending upon the wavelength(s), temporal characteristics (e.g., continuous versus pulsed delivery), and fluence of the EMR; the geometry, incidence and focusing of the EMR beam; and the index of refraction, absorption coefficient, scattering coefficient, anisotropy factor (the mean cosine of the scattering angle), and the configuration of the tissue layers; and the presence or absence of exogenous chromophores and other substances, the islets can be variously-shaped volumes extending from the surface of the skin through one or more layers, or extending from beneath the surface of the skin through one or more layers, or within a single layer.

Micro-islets may extend relatively deeply into the tissue, for example, from the surface of the skin into the subcutaneous fat layer. There are several mechanisms available to create relatively deep micro-structures. For example, a device may have one or more of the following features: an optical system designed for irradiating tissue below the surface; a mechanism to adjust the focus deeper into the tissue as the micro-structure is formed; a high-aspect ratio; and a relatively longer focal length. Other mechanisms that may be employed include, without limitation, delivery of EMR via a micro-fiber that is inserted into the microstructure as it is sized to essentially form a channel or tunnel in the tissue during the ablation process; local freezing of tissue that is to be ablated; and mechanical stretching of the skin to decrease density and increase EMR penetration.

In other embodiments, repeated pulsing that ablates a sub-volume of tissue from the micro-structure during the ablation process. However, when a single pulse of EMR is applied in a system, for example, aligned such that a focal area of the EMR is at or just below a tissue surface, multiple pulses of energy will gradually have less intensity deeper in the tissue as the beam diverges (as shown in FIG. 2). At some point, the intensity at a given depth will not exceed the threshold of ablation, as discussed in conjunction with FIG. 3. Thus, if such a system is used to create micro-holes that extend more deeply, additional mechanisms may be used in conjunction with multiple pulses, such as those discussed in the prior paragraph.

If multiple pulses are used to create a micro-structure, the pulses can be timed to allow the following pulse to be most effective. For example, the parameters may be selected to create a shock wave that temporarily expands a micro-hole during ablation, and, in some embodiments, the second pulse may be ideally timed to occur when the micro-hole is expanded, especially in embodiments where the scattering effects of ablated material within the micro-holes can be used advantageously to create particular shapes or dimensions within the micro-holes.

Furthermore, negative pressure may be applied to the tissue during the formation of a micro-islet, which will decrease the temperature of vaporization of the tissue. Negative pressure can also be used to modulate or control the process of formation of all micro-structures, both shallow and deep, including the width, depth, and shape of the micro-structure. For example, decreasing the pressure will decrease the temperature at which tissue is ablated, while increasing the temperature will increase the temperature at which tissue is ablated. Thus, for example, by modulating the pressure during the formation of the microstructure, the amount of tissue that is ablated per pulse can be changed.

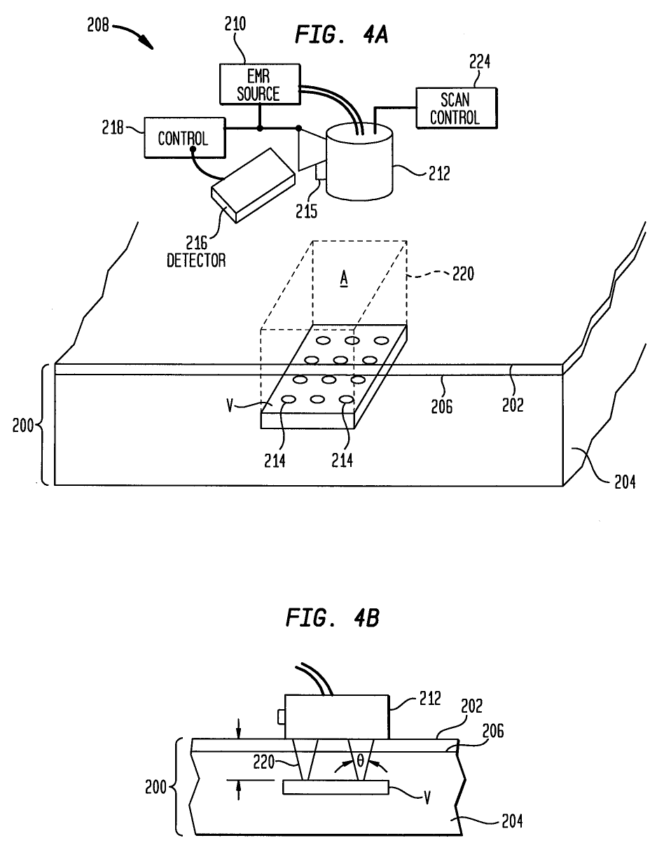

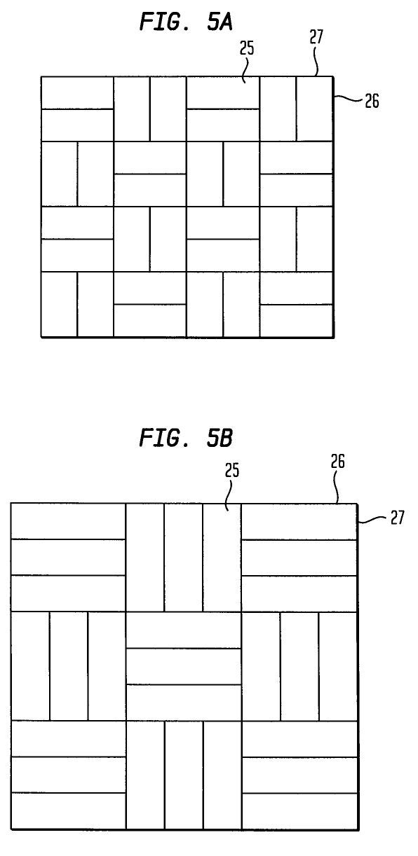

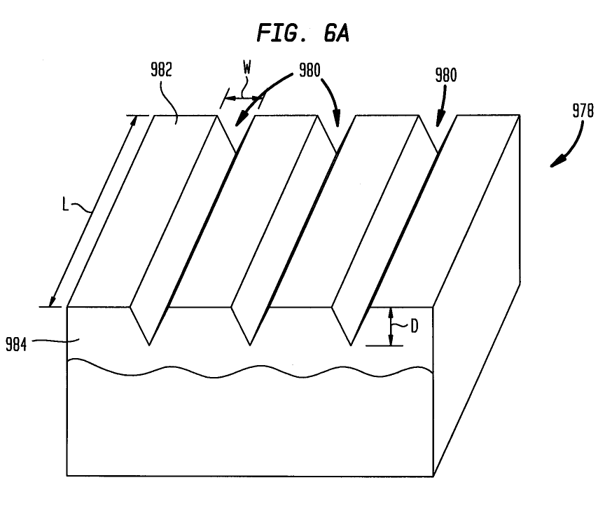

Referring to FIG. 4A, each of the treated volumes can be a relatively thin disk, as shown, a relatively elongated cylinder (e.g., extending from a first depth to a second depth), or a substantially linear volume having a length which substantially exceeds its width and depth, and which is oriented substantially parallel to the skin surface. The orientation of the lines for the islets 214 in a given application need not all be the same, and some of the lines may, for example, be at right angles to other lines (see for example FIGS. 5A and 5B). Lines also can be oriented around a treatment target for greater efficacy. For example, the lines can be perpendicular to a vessel or parallel to a wrinkle. Islets 214 can be subsurface volumes, such as spheres, ellipsoids, cubes or rectanguloids of selected thickness. The islets can also be substantially linear or planar volumes. The shapes of the islets are determined by the combined optical parameters of the beam, including beam size, amplitude and phase distribution, the duration of application and, to a lesser extent, the wavelength.

The parameters for obtaining a particular islet shape can be determined empirically with only routine experimentation. For example, a 2790 nm laser operating with a low conversion beam at approximately 0.005-2 J and a pulse width of 0.5-2 millisecond, can produce a generally cylindrically shaped islet. Alternatively, a 2940 nm laser operating with a highly converting beam at approximately 0.5-10 J and a pulse width of 0.5-2 millisecond, can produce a generally ellipsoid-shaped islet.

D. Grooves and Micro-Grooves

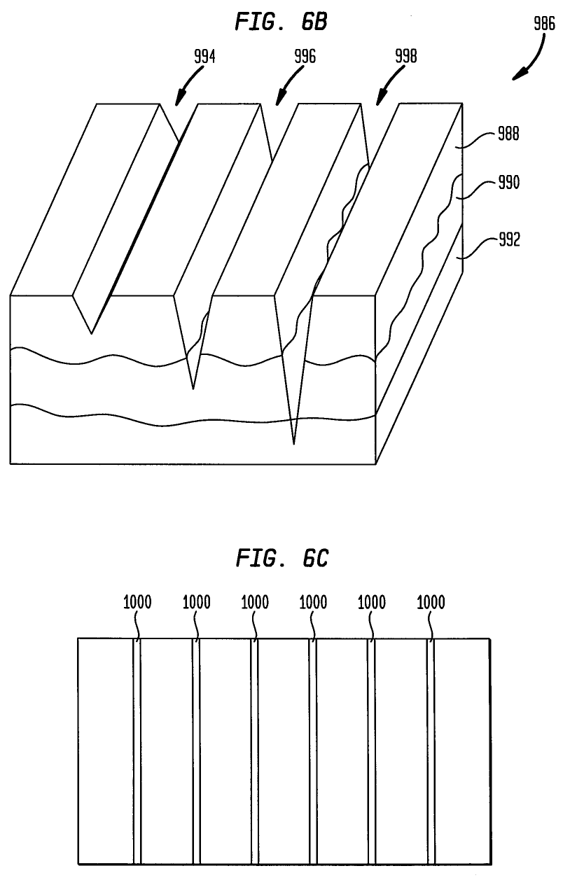

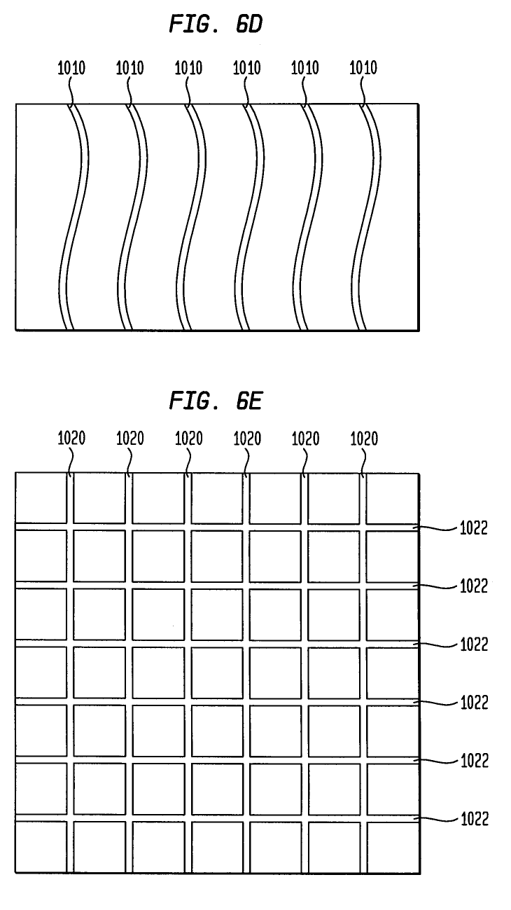

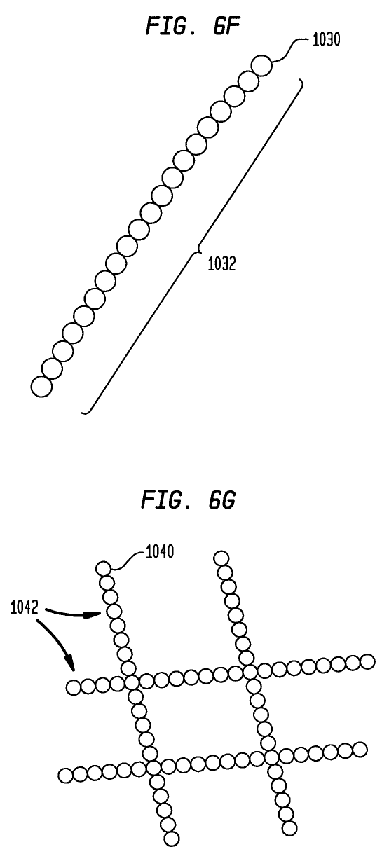

One form of an ablation islet that is particularly useful in certain applications is an ablated groove extending in a row some distance along the surface of the skin tissue. In particular, the ablated groove may be a micro-groove. For example, referring to FIG. 6A a section of skin tissue 978 is shown containing three ablated micro-grooves 980 that extend from a skin surface 982 into an epidermis 984 of the tissue 978. Each micro-groove 980 has a length (“L”), a width (“W”), and a depth (“D”). For example, the length L of each micro-groove 980 is 2 cm while the width W of each micro-groove is approximately 100 μm and the depth is approximately 150 μm. Many dimensions and shapes are possible, however. Exemplary ranges are lengths L of 500 μm to several feet, while widths may be from 10 μm to 500 μm or more preferable between 30 μm and 100 μm. Larger groove widths are possible, but the potential for bulk tissue damages increases with larger structures. Thus, as discussed below, micro-grooves are preferred over larger groove structures in most applications.

Grooves may also have a range of depths. For example, referring to FIG. 6B, a tissue section 986 includes an epidermal layer 988, a dermal layer 990 and a portion of a subcutaneous fat layer 992. Tissue section 986 further includes three micro-grooves 994, 996 and 998. Micro-groove 994 extends from a skin surface into the epidermis; micro-groove 996 extends from a skin surface into the dermis; and micro-groove 998 extends from a skin surface into the subcutaneous fat tissue. An exemplary range of depths for various micro-grooves is 100 μm to 5 mm, but other depths are possible depending on the application.

The fill factor (discussed in more detail below) can be from about 1% to about 90% and more preferably from about 1% to about 50%.

Groove structures may also take on many shapes and patterns. For example, referring to FIGS. 6C, 6D and 6E, arrays of micro-grooves illustrate regularly patterned and irregularly patterned arrays. Micro-grooves 1000 are straight and parallel rows as shown in FIG. 6C; micro-grooves 1010 are regularly-spaced curved rows or furrows as shown in FIG. 6D, and micro-grooves 1020 and 1022 are intersecting rows or furrows as shown in FIG. 6E. The grooves may be V-shaped as illustrated or have many different alternative configurations, including, without limitation, a U-shaped trough, a circular-shaped trough, a rectangular shape, a cross-section that is wider at the base of the groove than at the opening or surface of the groove, or a relatively narrow neck with a larger void below the opening or surface of the groove.

Furthermore, grooves can be formed by a number of different mechanisms. For example, a micro-groove can be formed by a single beam continuously scanned along a path to ablate tissue to form a groove along that path. Micro-grooves can be formed using a phase array. A cylindrical lens or similar lens may be used to focus EMR along a path on the tissue where the groove will be formed. Additionally, as shown in FIGS. 6F and 6G, a set of pulses of EMR may be generated either sequentially or simultaneously to form a set of spots 1030 and 1040 on the tissue. When tissue is ablated at the spots, the result is a single groove 1032 or a set of grooves 1042. In still other embodiments, the grooves may be circles, semicircles, and concentric circles or semi-circles. Additionally, combinations of grooves and other micro-structures or types of EMR-treated islets (both ablative and non-ablative) can be used, such as micro-holes in combination with a circular micro-groove or a damage EMR-treated islet in between intersecting grooves. Many other embodiments are possible, including other shapes, patterns, dimensions and combinations.

E. Fill Factor

In a given lattice of EMR-treated islets, the percentage of tissue volume which is EMR-treated is referred to as the “fill factor” or f, and can affect whether optical islets become thermal islets, damage islets or photochemical islets. The fill factor is defined by the volume of the islets with respect to a reference volume that contains all of the islets. The fill factor may be uniform for a periodic lattice of uniformly sized EMR-treated islets, or it may vary over the treatment area. Non-uniform fill factors can be created in situations including, but not limited to, the creation of thermal islets using topical application of EMR-absorbing particles in a lotion or suspension (see below). For such situations, an average fill factor (f_{avg}) can be calculated by dividing the volume of all EMR-treated islets Vᵢ^{islet} by the volume of all tissue Vᵢ^{tissue} in the treatment area,

$${f}_{avg} = {\sum }_{i} \frac{{V}_{i}^{islet}}{{V}_{i}^{tissue}} . \qquad ( 2 )$$

Generally, the fill factor can be decreased by increasing the center-to-center distance(s) of islets of fixed volume(s), and/or decreasing the volume(s) of islets of fixed center-to-center distance(s). Thus, the calculation of the fill factor will depend on volume of an EMR-treated islet as well as on the spacing between the islets. In a periodic lattice, where the centers of the nearest islets are separated by a distance d, the fill factor will depend on the ratio of the size of the islet to the spacing between the nearest islets d. For example, in a lattice of parallel cylindrical islets, the fill factor will be:

$$f = {π ( \frac{r}{d} )}^{2} , \qquad ( 3 )$$

where d is the shortest distance between the centers of the nearest islets and r is the radius of a cylindrical EMR-treated islet. In a lattice of spherical islets, the fill factor will be the ratio of the volume of the spherical islet to the volume of the cube defined by the neighboring centers of the islets:

$$f = \frac{4 π}{3} {( \frac{r}{d} )}^{3} , \qquad ( 4 )$$

where d is the shortest distance between the centers of the nearest islets and r is the radius of a spherical EMR-treated islet. Similar formulas can be obtained to calculate fill factors of lattices of islets of different shapes, such as lines, disks, ellipsoids, rectanguloids, or other shapes.

Because untreated tissue volumes act as a thermal sink, these volumes can absorb energy from treated volumes without themselves becoming thermal or damage islets. Thus, a relatively low fill factor can allow for the delivery of high fluence energy to some volumes while preventing the development of bulk tissue damage. The lattice thermal relaxation time (LTRT) may be defined as the characteristic cooling time when the maximum temperature within the islet reaches the intermediate value between the initial and stationary temperatures. Using this definition the LTRT of a very sparse lattice equals the thermal relaxation time (TRT) of an individual islet. Actually, for such a lattice each islet cools independently on the others. For denser lattices the temperature profiles from different islets overlap causing the LTRT to decrease. To estimate such cooperative effect, the ratio of LTRT to TRT as a function of the fill factor (f) for the particular case of the 2D lattice was calculated (FIG. 4). The LTRT decreases monotonically with the growth of the fill factor. Therefore, the denser is the islet lattice the smaller is the time while the lattice relaxes by coming down to the thermal equilibrium with the surrounding tissue. When the fill factor approaches unity, the LTRT approaches some limit close but somewhat larger than the TRT. As an estimate (where f>=0.1):

$$\frac{LTRT}{TRT} \approx \frac{1}{3 \cdot f} , \qquad ( 5 )$$

Finally, because the untreated tissue volumes act as a thermal sink, as the fill factor decreases, the likelihood of optical islets reaching threshold temperatures to produce thermal islets or damage islets also decreases (even if the EMR power density and total exposure remain constant for the islet areas).

The center-to-center spacing (i.e., pitch) of islets is determined by a number of factors, including the size of the islets and the treatment being performed. Generally, it is desired that the spacing between adjacent islets be sufficient to protect the tissues and facilitate the healing of any damage thereto, while still permitting the desired therapeutic effect to be achieved. In general, the fill factor can vary in the range of 0.1-90%, with ranges of 0.1-1%, 1-10%, 10-30% and 30-50% for different applications. The interaction between the fill factor and the thermal relaxation time of a lattice of EMR-treated islets is discussed in detail below. In some embodiments producing thermal islets, the fill factor may be sufficiently low to prevent excessive heating and damage to islets. In some embodiments producing damage islets, the fill factor may be sufficiently low to ensure that there is undamaged tissue around each of the damage islets sufficient to prevent bulk tissue damage and to permit the damaged volumes to heal. The specific parameters, such as the degree of separation and the ratio of the volume of islets to the volume of tissue that is treated but in which islets are not formed, will vary depending on the application. In some embodiments, for example, the entire treated tissue could be irradiated to some degree, such as to cause a thermal reaction in the tissue or a degree of damage in the tissue while the EMR-treated islets would be formed within that tissue and would have a greater degree of damage. For example, a lattice of damage islets could be formed within a volume of tissue that has been treated to provide an underlying bias of heat throughout the volume of tissue. As another example, a lattice of islets of ablative damage could be provided in a tissue volume that has been damaged to a lesser degree. Such an embodiment may be useful, for example, to create holes or channels in damaged fat tissue to insert or extract substances or for other purposes.

### II. Devices and Methods For Creating Micro-Holes and Other EMR-Treated Islets

A. Products and Methods for Ablating Tissue

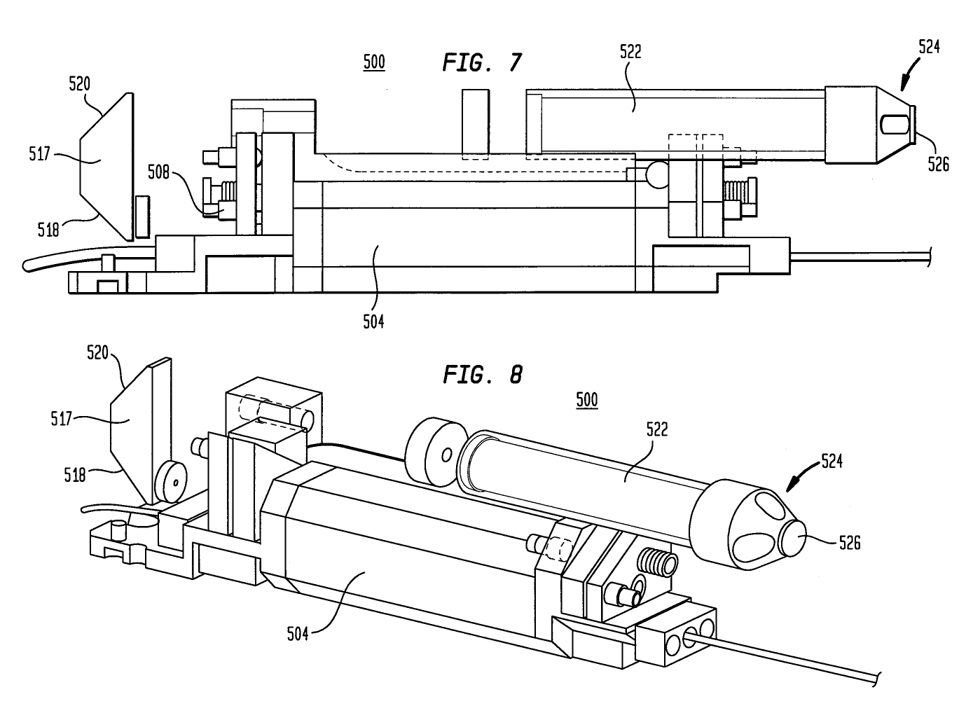

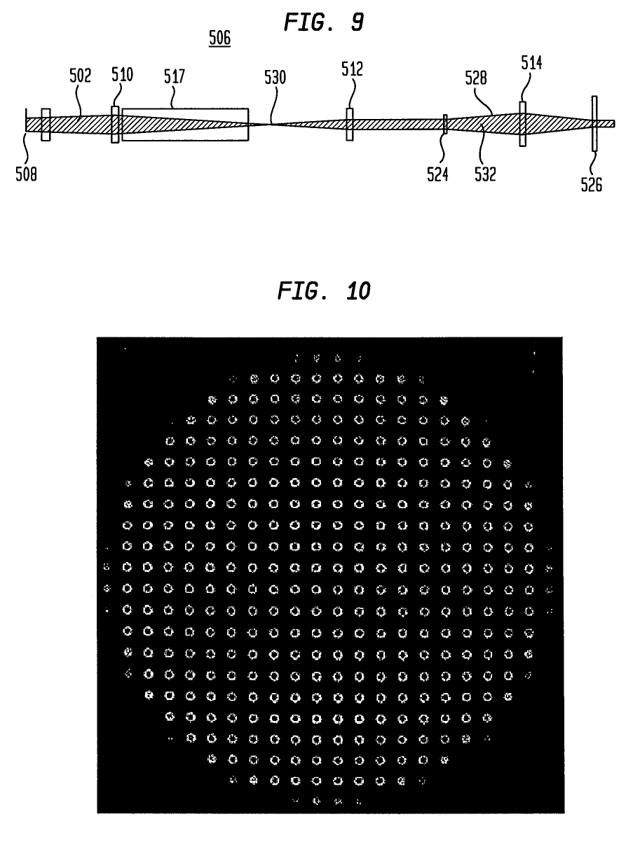

In one embodiment, referring to FIGS. 7-9, an ablation device 500, designed to ablate the surface of the skin or other tissues, passes electromagnetic radiation 502 from a radiation source 504, along an optical assembly 506 and out a radiation window 508 at an end of optical assembly 506.

Electromagnetic radiation 502 can be any radiation useful for ablating tissue, and, in this embodiment, is electromagnetic radiation having a wavelength of approximately 2940 nm (nanometers). Other wavelengths that are particularly useful in other embodiments similar to that shown in FIGS. 7-9 are wavelengths in the range of 2000 to 3500 nm and more particularly in the range of 2500 to 3100 nm. However, many other wavelengths can be used, including

In device 500, electromagnetic radiation (“EMR”) 502 is produced by radiation source 504, which in the present embodiment is a Q-switched YGG:YAG laser. However, any mechanism for producing EMR at the desired wavelength, power and duration may be used, including other lasers, flashlamps, other lamps, and other sources of EMR. Electromagnetic radiation 502 is emitted from an end 508 of radiation source 504.

Electromagnetic radiation 502 travels through optical assembly 506. Optical assembly 506 includes first, second, and third lenses 510, 512, and 514, prism 517, and transmission tube 522. Transmission tube 522 has a lens array 524 and an aperture 526 that serves as an opening through which the beams of EMR are transmitted. In operation, device 500 functions as a laser handpiece that is made relatively more compact by folding the path that electromagnetic radiation 502 back on itself via a 180 degree turn. Electromagnetic radiation 502 is emitted from radiation source 504 and passes through first lens 510. First lens 510 is a convergent imaging lens that focuses EMR 502 into prism 517. EMR 502 strikes a first reflective end 518 of prism 517. End 518 is oriented at an angle of 45 degrees relative to the line of travel of electromagnetic radiation 502, and causes electromagnetic radiation 502 to be reflected (via total internal reflection within prism 517) at a 90 degree angle toward a second reflective end 520 of prism 517. Second end 520 similarly is oriented at an angle of 45 degrees relative to the line of travel of electromagnetic radiation 502, and causes electromagnetic radiation 502 to be reflected again at a 90 degree angle toward and through transmission tube 522.

Many alternate embodiments are possible to achieve the result, including, for example, the use of reflective materials, coatings, and/or mirrors. Similarly, depending on the design considerations, other embodiment may have other configurations for the path that EMR travels, such as, for example, a straight path with no turn, an “L”-shaped path or other configurations. Similarly, the EMR could travel along an optical fiber from a source, which could be located in a handpiece, in a base unit, or other configuration.

After EMR 502 exits prism 517, EMR 502 is focused through focal spot 530 and begins to diverge. EMR 502 then travels through second lens 512, which is a convergent lens that makes the beam of EMR 502 less divergent after is exits prism 517. EMR 502 diverges until it reaches lens array 524. At that point, EMR 502 has a perpendicular cross-section that is circular in shape and that is smaller than the area of lens array 524, which is approximately square in shape. Lens array 524 is an array of micro-lenses that focus EMR beam 502 into an array of beams 528. One suitable lens array is manufactured by SUSS MicroOptics SA, #112-0571. Lens array 524 produces 770 beams each having a pitch of 360 microns and a beam diameter of 110 micrometers per beam. Lens array 524 produces a pattern of EMR-treated islets as shown in FIG. 10. The beams have a density of 770 beams per cm².

The array of beams 528 then travels through third lens 514. Lens 514 is a convergent imaging lens that re-images the array of beams along an imaging plane that corresponds to the location of an aperture 526. The imaging plane (and aperture 526) are located approximately 27 mm from lens 514.

Aperture 526 is a grating or mesh consisting of a metal surface having holes aligned with the position of the beams in the array 528. The holes of aperture 526 are configured as shown in FIG. 10. The holes in the grating allow the beams in the array 528 to pass to the tissue, while allowing the solid portion of the surface to press against the tissue such that it remains flat. By flattening the tissue, the aperture ensures that the tissue is at a uniform distance from the third lens 514, thereby improving the uniformity and precision of the energy delivered to the tissue. However, other configurations are possible, such as a transparent window having appropriate optical parameters that presses against the tissue to flatten it. Additionally, other embodiments could be curved or include additional elements such as a vacuum or mechanical device to stretch or otherwise move the skin.

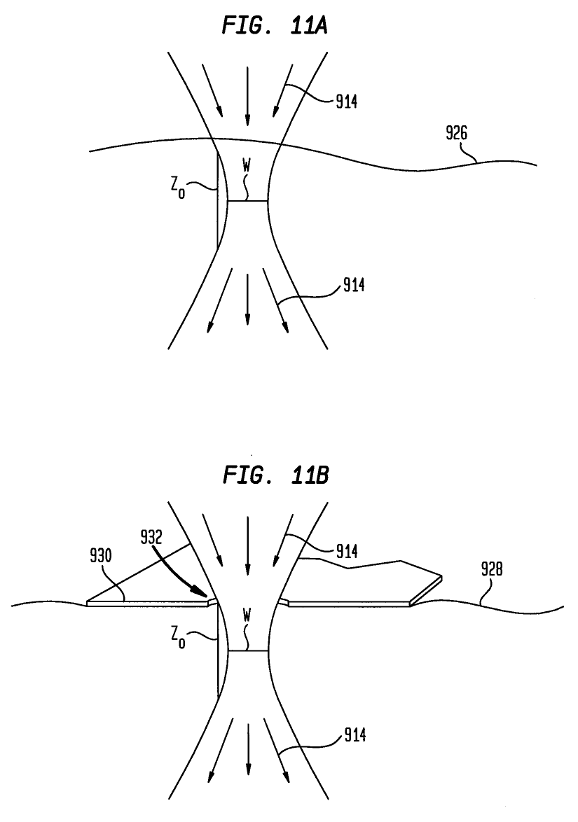

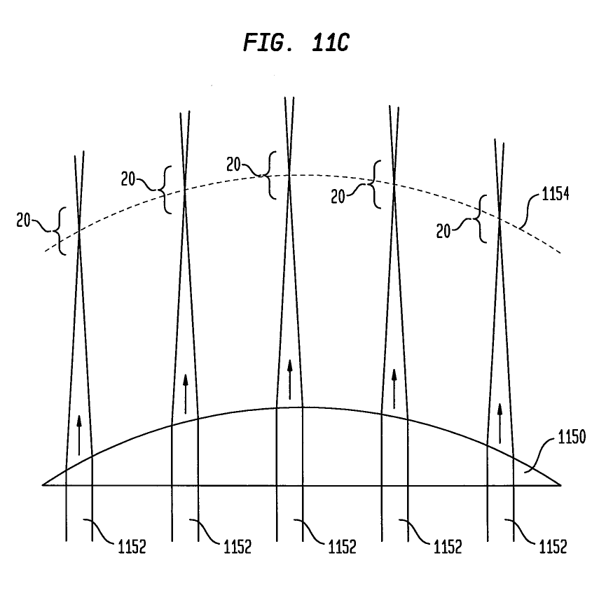

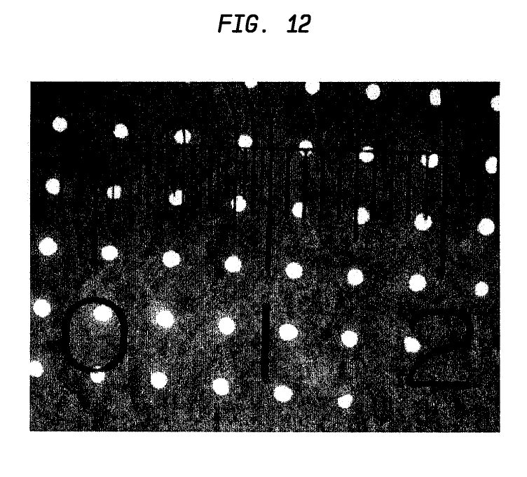

The use of the aperture and/or other mechanisms such as stretching the skin or conforming the skin surface to another surface allows the device to uniformly irradiate an area of tissue with an array of beams. Thus, the use of such a device improves the precision of the device and allows it to create even smaller holes on a consistent basis. For example, referring to FIG. 11A, even a relative flat area of skin will have significant variations in surface terrain 926 that can affect the alignment of the tissue surface relative to the focal plane W of one or more beams relative to the skin surface. The fluctuations in the surface terrain of the tissue will begin to be a greater percentage of the focal depth for smaller micro-holes. Thus, the variation in skin surface terrain will have a greater impact as the size of the diameter of the micro-holes decreases. Again (as discussed in conjunction with FIG. 3), a smaller micro-hole is made by decreasing the diameter of the focal area which exponentially reduces the focal depth. Thus, as the diameter of the micro-holes are reduced to very small sizes, the range of the focal depth is decreased and the margin in which the skin or other tissue surface is aligned with the volume of maximum intensity of EMR decreases. Although it is not essential that the skin surface be aligned within the range of the focal depth Z₀, it is preferable in some embodiments to align the skin within that range to more precisely control the formation of micro-holes.

By using a mesh grating with an even surface (which can be flat or contoured), the surface tissue can be precisely aligned with the focal plane of the beams to allow uniform micro-holes to be created. For example, referring to FIG. 11B, a portion of an aperture 930 is shown. The aperture contains an array of holes 932 through which EMR may pass. In FIG. 11B, one hole 932 is shown. The light rays 914 from a single beam are focused to a focal point having a diameter W of, e.g., 30 μm. In operation, the exterior surface of aperture 930 presses against tissue surface 928 and flattens it. In doing so, aperture 930 aligns the tissue surface near the upper boundary of the focus depth Z₀. Although other alignments are possible, this alignment of the tissue relative to the focus depth allows nearly the entire length of the focal depth to lie within the tissue, which allows the portion of the beam that has the greatest intensity to be directly incident on the tissue. With this alignment, the parameters of the system can be chosen such that only the portion of the beam within the focal plane has sufficient intensity to ablate or otherwise damage the tissue.

In an alternative embodiment, the surface of an aperture is curved to conform to optical characteristics of an optical system. For example, if an array of beams is imaged with an imaging lens, the focal plane of the imaging lens will have a contour that is not flat. In that case, the alignment device can be contoured to match the focal plane produced by the optical system to allow the tissue to be aligned precisely with the focal plane of the device. Referring to FIG. 11C, and exemplary imaging lens 1150 focuses an array of beams 1152 onto a focal plane 1154. The focal plane is not flat. Thus, to precisely align the tissue, the alignment device or surface should conform the surface of the tissue to the contour of the focal plane, preferably (but not necessarily) such that the tissue surface is aligned within the focal depths Z₀ of each beam 1152.

In some embodiments, the beam can have an intensity such that the ability to ablate, damage or otherwise treat the tissue extends to a portion that is less than or greater than the length of the focal depth. Furthermore, it should be noted that, regardless of the intensity of the beam, a micro-hole can be increased in size, including depth, by firing multiple pulses of EMR. Additionally, if required, the focal point can be adjusted, e.g., by repositioning the focal plane deeper into the tissue between pulses or in a continuous fashion during the pulse, or during the application of EMR, for example, if quasi-continuous wave or continuous wave modes of operation are used.

While such an aperture or similar structure is expected to produce superior results when forming small micro-holes, such a device or structure is not required. For example, a higher intensity pulse can be used to create micro-holes in embodiments where the variations in the tissue surface terrain exceed the focal depth of the device. Thus, in other embodiments, no such aperture, window or similar mechanism to ensure the uniformity of the distance of the optical elements to the tissue to be treated is included. However, when attempting to precisely create uniform holes on the order of approximately 50 μm or less, the better practice is thought to be to align the surface of the tissue to a uniform distance using a device or structure such as aperture 526 (or another device or structure that aligns the surface to the desired distance).

In operation, the surface of the tissue to be ablated will be pressed against aperture 526, and the array of beams will ablate the surface of the tissue. In the present embodiment, a safety mechanism such as a contact sensor preferably is included to prevent the laser from firing when the tissue is not in contact with the aperture 526. That will prevent, among other things, the condition where the laser is accidentally fired while the aperture 526 is off the surface of the tissue. (Many other configurations are possible. For example, an alternative optical assembly could result in the beams exiting the device in a parallel or a slightly divergent orientation, to prevent the array of beams from being applied to the surface at a greater intensity, thereby potentially damaging the tissue to an excessive degree) due to the convergence of the beams at the exit the device.)

During operation, referring again to FIGS. 7-9, lens array 524 focuses the radiation having a wavelength of approximately 2940 nm in an orthogonal pattern within a generally circular treatment region (or footprint on the treated tissue). The radiation is applied at a fluence of 5.5 mJ (millijoules) using a pulse width of 200 microseconds and a repetition rate of 0.5 Hz. Typically, a suitable optical impedance matching lotion or other suitable substance would be applied between aperture 526 and the tissue being treated to provide enhanced optical and thermal coupling, but such a lotion is not required.

FIG. 12 is an example of the pattern produced by device 500. In this case, the pattern was produced by irradiating one side of a piece of paper, which was photographed from the opposite side. The units of measure in the photograph are in millimeters.

Many other patterns, such as, for example, hexagonal, rectangular, circular, triangular, etc., could also be used. The various patters have different advantages. For example, a hexagonal pattern would be preferable for providing greater beam densities, while an orthogonal pattern allows comparatively greater regions of untreated tissue between the volumes of treated tissue and/or allows relatively larger beam diameters. Additionally, the pattern need not be uniform and patterns created by beams having varying relative cross-sectional areas and shapes can be used alone or in combination.

Many other embodiments are possible. For example, the specifications for devices similar to device 500 can include those listed in Table A below (although the specifications are exemplary only of such embodiments, and do not encompass all possible embodiments or all possible operating parameters for devices similar in structure to device 500).

TABLE A
Potential Operating Ranges For Certain Embodiments

| Parameter | Range |
|---|---|
| Wavelength | 2100-3100 nm, particularly 2690 nm, 2790 nm, |
| | 2810 nm and 2940 nm |
| Output Energy | Up to 3 J total, particularly 1.3 J total |
| Pulse Duration | 0.1-10,000 microseconds, particularly 200 |
| | microseconds |
| Beam Diameter | 10-200 micrometers, particularly 110 micrometers |
| Beam Density | 100-12,000/cm², particularly 770/cm² |
| Beam Pattern | Orthogonal or Hexagonal |
| Beam Pitch | 100-1,000 micrometers, particularly 360 micrometers |
| Energy per Beam | 0.1-50 J/cm², particularly 10 J/cm² |
| Repetition Rate | Single pulse, 0.1-10 Hz, particularly 0.5 |

Furthermore, in still other embodiments, the EMR from the energy source can be focused by an optical device and/or shaped by masks, filters, optics, or other elements in order to create islets of treatment on the subject's skin. In some embodiments, components found in device 500 may not be present, such as, for example, prism 517 or lens array 524. Other embodiments could include different combinations, types and number of optical components. Other embodiments could be configured to irradiate the tissue without the device being in contact with the tissue or by having an offset or spacer that spaces a transmission opening or other source of radiation some distance from the surface of the tissue during operation. In yet another embodiment, there is no cooling mechanism such that there is only passive cooling between the contact plate and the skin.

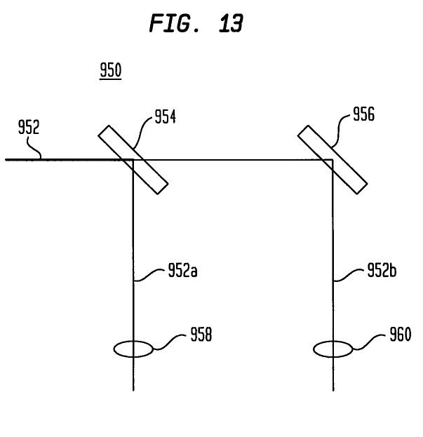

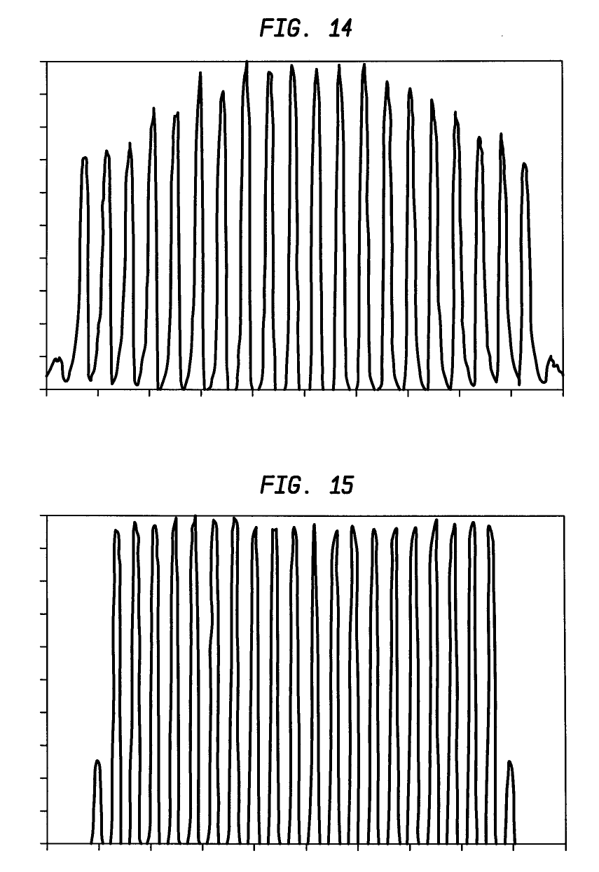

Additionally, other embodiments could include mechanisms other than lens arrays, such as scanning devices, partially reflective mirrors, etc. For example, one alternate embodiment could include a scanner that uses a single beam or several beams repeatedly to create the columns of damage in the tissue. Similarly, referring to FIG. 13, an array of mirrors 950 could be used. In this particular embodiment, a beam of EMR 952 passes through a set of mirrors that create a set of sub-beams 952a and 952b. EMR 952 passes through a first mirror 954 oriented at an angle of 45 degrees relative to the path of the EMR 952. Mirror 954 reflects 50 percent of EMR 952 at a ninety degree angle to form sub-beam 952a and allows the remaining portion of EMR 952 to pass through mirror 954 to create sub-beam 952b. Sub-beam 952b travels to a second mirror 956 that is 100 percent reflective. Second mirror 956 also is oriented at an angle of 45 degrees relative to the path of the EMR 952 and reflects sub-beam 952b at a ninety degree angle and parallel to sub-beam 952a. Both beams travel through lenses 958 and 960 respectively. Lenses 958 and 960 focus the sub-beams 952a and 952b onto the tissue. Although the present embodiment creates two sub-beams, many different configurations and combinations of configurations are possible.

Furthermore, the characteristics of the resulting columns can be controlled by modulating the pulses of the beams that are applied to the tissue. This can be done, for example, spatially or temporally. In some embodiments, the spatial geometry of the beams can be designed to create resulting columns having specific characteristics. In other words, by varying the geometry of the beams, including the overall pattern, the shape of the individual beams and/or the combination of differently shaped beams, the dimensions and other characteristics of the resulting columns of damage in the treated tissue can be controlled. For example, by increasing the relative cross-sectional area of the individual beams, the depth of the columns into the tissue can be increased.

As another example, the shape of the footprint that the EMR islets form on the tissue can be varied to suit a particular application. For example, the footprint of the array of beams 528 in device 500 is circular. There are various methods to control the shape of the footprint. In a scanning system, the system can be programmed to direct the beam in a pre-designated pattern. Similarly, in embodiments using an optical imaging system similar to that of device 500, the beam of EMR can be conditioned prior to passing through the lens array to have the desired cross-sectional shape.

One potential design consideration is the amount of blurring that occurs in the periphery of the array of beams 528. For example, in tests using device 500, some degree of blurring of individual beams occurs in the periphery of the array of beams 528. The blurring, which is illustrated in FIG. 10, is due to the fact that the optical assembly 506 is designed to image the beam of EMR 502. In such imaging systems, blurring increases with the distance from the center of the beam. Device 500 is designed to keep such blurring within acceptable limits such that each of the individual beams remain effective without unduly increasing the cost of the device by attempting to optimize the system to ideal parameters. In alternate embodiments, such blurring can be reduced or eliminated through other means, for example, by using a design having a optical path that is optimized to an even greater degree (although this may be more expensive), collimating the beam of EMR into parallel rays prior to passing the EMR through the lens array, or using a scanning system that reflects a single beam of EMR into various locations to form the islets of damage. Other embodiments are possible.

Referring to FIGS. 14 and 15, computer simulations of two sets of beams are compared. The relative intensity of the beams are plotted along the vertical axis. The horizontal axis measures the position of the beams through a center line that is perpendicular to the direction of the beams. The beams shown in FIG. 14 are similar to the distribution of the beams shown in FIG. 10. The array of beams have a Gaussian-like distribution in which the intensity of the beam decreases as a function of position from the center of the array. This decrease is due to the limitations of the optics that are used. If more precise optics are used and/or the beams are further processed, such as, for example, by collimating the beams, the distribution of the beam becomes more uniform, which will improve the optical quality of the beams that lie towards the periphery of the array. FIG. 15 is a graphical depiction of such beams based on a computer simulation. In FIG. 15, the horizontal and vertical axes depict the same information as in FIG. 14. There, the conditioned beam produces beams that have nearly equal intensities across the majority of the beam with a decrease in intensity only at the extreme edges of the array.

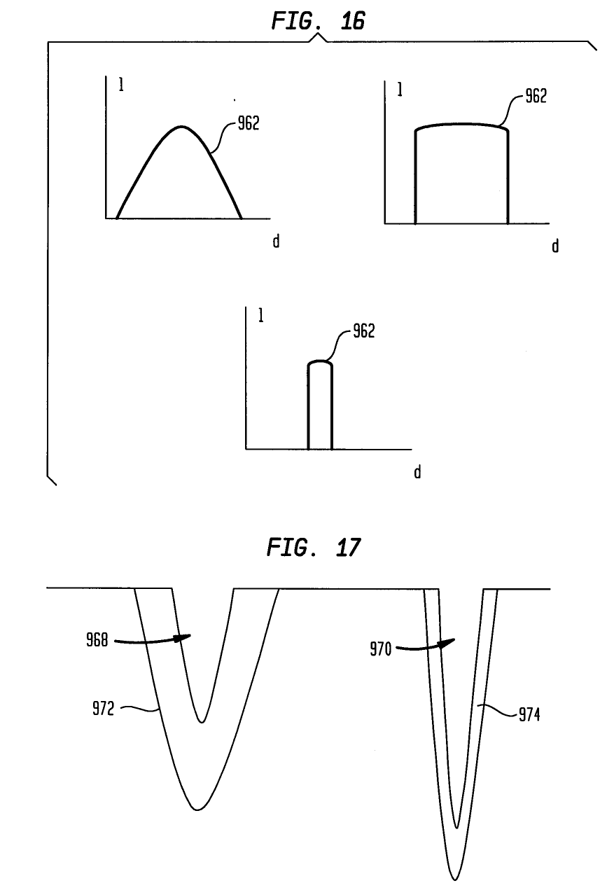

Referring to FIG. 16, if a beam of EMR 962 in an optical imaging system is not conditioned prior to passing through a micro-lens array, it likely will have a generally Gaussian distribution of intensity I of EMR relative to the position d within the cross-section of the beam of EMR. That is, the cross-section of the beam of EMR likely will be circular, but the intensity of the beam will not be uniform. Instead, the intensity of the beam will be greater in the center than at the edges. Such a device would tend to cause uneven applications of EMR on the surface of the tissue being treated, and could potentially burn one portion of the treated area and/or inadequately treat the periphery of the treated area. Thus, it may be preferable to condition the beam that forms the array of beams with optical elements, such as waveguides, lenses, and/or filters, etc. to provide a more uniform beam, such as beams 964 and 966, which are roughly rectangular and have a more uniform distribution of intensity. Such conditioning may not be required if, for example, multiple sources are used, such as an array of laser diodes in which each diode forms a separate beam.

In still other embodiments, additional sensing devices can be employed to control the treatment parameters. For example, referring again to FIGS. 7-9, device 500 could be equipped with a hydration sensor to monitor the amount of moisture in the tissue being treated. Ablating tissue with EMR having a wavelength above 2000 nm tends to be more efficacious than treating with EMR in lower ranges. However, there is a greater chance of damaging the tissue or inducing side-effects in these ranges. Additional precautions could be taken to improve the overall safety of such devices, such that they are as safe as devices using shorter wavelengths. One such precaution could be monitoring the moisture level in the tissue to ensure that the surface of the tissue does not become too dry, which would make the tissue more susceptible to damage. The device can also be equipped with a reservoir to apply a moisturizing fluid, such as water, lotion or appropriate fillers.

Still other embodiments can have a hyperbaric chamber in communication with the tissue to apply substances, for example, oxygen to help the wound healing process. In still other embodiments, a vacuum chamber can be provided that is used to clean the micro-hole of debris. In still other embodiments, combinations of capabilities are combined to, for example, clean the micro-holes and administer a substance to promote healing before, during or after treatment.

### B. Use of Various Wavelengths and Modulation of Wavelengths

In various embodiments, additional or other lasers or other EMR sources can be used to produce EMR of other wavelengths. In the case of non-coherent sources, various mechanisms can be used including the use of one or more filters, including adjustable or replaceable filters that allow the wavelength to be changed. In the case of coherent EMR sources, a tunable source can be used. When lasers specifically are used, the lasing medium may be altered, e.g., by employing different mediums and/or adjusting the doping of the lasing medium. For example, the following wavelengths could be used in other embodiments:

| Laser Type | | Wavelength |
|---|---|---|
| CTH: YAG | (yttrium aluminium garnet) | 2690 nm |
| Cr: Er: YAG | (yttrium aluminium garnet) | 2690 nm |
| YSGG | (yttrium scandium gadolinium garnet) | 2790 nm |
| YLF | (yttrium lithium fluoride) | 2890 nm |
| YGG: YAG | (yttrium gadolinium garnet) | 2940 nm |

The above table is exemplary only, and the various types and concentrations of dopants for the laser crystals that will produce various wavelengths are understood by those skilled in the art. Many other laser types and configurations are possible, including potentially other solid-state lasers as well as gas, eximer, dye, tunable, semiconductor and other types of lasers. Furthermore, other wavelengths could be generated using an optical parametrical oscillator to generate EMR having a wavelength in the range of approximately 2500-3100 nm as well as to generate even longer wavelengths, for example, by manipulating EMR at a particular wavelength, e.g., 690 nm, to generate EMR having a wavelength that is twice as long, e.g., 1380 nm. (This is similar to the concept of frequency doubling or tripling in which EMR of a particular wavelength, e.g., 1040 nm, is manipulated to generate EMR having a shorter wavelength, e.g., 520 nm.) Although certain wavelengths and combinations of wavelengths will be advantageous in particular applications, essentially any wavelength of EMR can be used. However, wavelengths above 0.29 μm are preferred due to the potentially hazardous impact smaller wavelengths may have on human tissue in vivo.

In still others embodiments, various sources or a tunable source can be used to modulate the parameters of the EMR that is applied, and, thus, control the resulting dimensions of the micro-holes that are created. In one such embodiment, wavelength can be modulated to control the dimensions of the micro-hole. Referring to FIGS. 17 and 18, different wavelengths of EMR will have different effects on the tissue to which they are applied. For example, EMR having a wavelength of 2650 nm has a coefficient of absorption in skin tissue of approximately 3,000 cm⁻¹. EMR having a wavelength of 2940 nm has a coefficient of absorption in skin tissue of approximately 10,000 cm⁻¹. Thus, when EMR at 2650 nm is used to produce a micro hole in the surface of the skin tissue, a resulting micro-hole 968 will be relatively shallower in comparison to a micro-hole 970 produced using EMR having a wavelength of 2940 nm, if the same power density is applied for the same amount of time. If a higher power density is applied or the EMR is applied for a longer period of time, the resulting micro-hole 968 may have a greater depth into the tissue, but the zone of damaged tissue 972 surrounding the micro-hole 968 will also increase. Thus, in comparison to micro-hole 970, the micro-hole 968 (including the surrounding zone of damaged tissue 972) resulting from the use of EMR at 2650 nm will tend to be more stout in shape than the micro-hole 970 (including the surrounding zone of damaged tissue 974) assuming that other parameters are the same or are similar.

This phenomenon can be used to control the shape of the resulting micro-holes. For example, referring to FIG. 18, the wavelength of EMR can be modulated to select the desired coefficient of absorption of the tissue being treated to control the dimensions of the resulting micro-hole. This can be done prior to applying the EMR or while applying the EMR. For example, a first pulse of EMR having a wavelength of 2650 nm can be applied to induce a larger damage zone. The wavelength can then be tuned to 2940 nm and a second pulse of EMR can be applied to create a deeper and narrower micro-hole. Also, the wavelength can be modulated continuously during application of EMR to various points on the curve, as illustrated by the arrows. Many other combinations are possible using the wavelengths shown in FIG. 18, other wavelengths and other parameters, such as power density, fluence, etc. The desired geometry and other characteristics of the resulting micro-holes will depend on the application, the type of tissue and other factors.

C. Alternate Embodiments For Creating Ablation Islets

In still other embodiments, the energy source may be any suitable optical energy source, including coherent and non-coherent sources, able to produce optical energy at a desired wavelength or a desired wavelength band or of multiple wavelengths or in multiple wavelength bands. For example, wavelengths that have complimentary physical characteristics can be used, such as one wavelength that is highly absorbed by a particular type of tissue in combination with or followed by a wavelength having a lower absorption, for example, to serve a hemostatic function and seal any bleeding blood vessels.

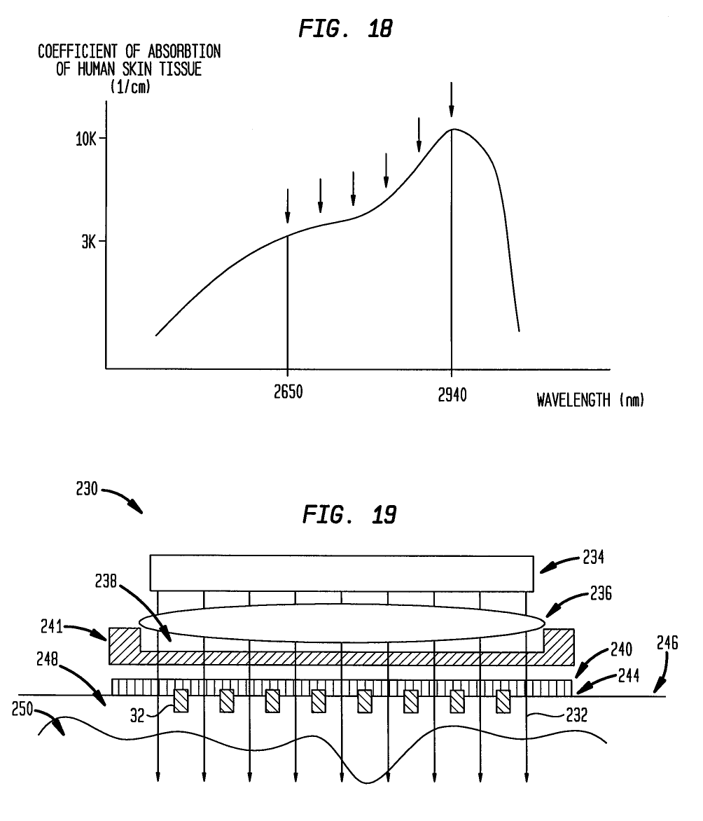

In another embodiment, FIG. 19 shows a broad overview schematic of an apparatus 100 that can be used to produce islets of treatment in the patient's skin. For this apparatus 230, optical energy 232 from a suitable energy source 234 passes through optical device 236, filter 238, cooling mechanisms 240, 242, and cooling or heating plate 244, before reaching tissue 246 (i.e., the subject's skin). Each of these components is described in greater detail below. Generally, however, the EMR from the energy source 234 is focused by the optical device 236 and shaped by masks, optics, or other elements in order to create islets of treatment on the subject's skin. In some embodiments, certain of these components, such as, for example, filter 238 where a monochromatic energy source is utilized or optics 236, may not necessarily be present. In other embodiments, the apparatus may not contact the skin. In yet another embodiment, there is no cooling mechanism 4 such that there is only passive cooling between the contact plate and the skin.

A suitable optical impedance matching lotion or other suitable substance would typically be applied between plate 244 and tissue 246 to provide enhanced optical and thermal coupling. Tissue 246, as shown in FIG. 19, is divided into an upper region 248, which, for applications where radiation is applied to the skin surface, would be the epidermis and dermis, and a lower region 250, which would be a subdermal region in the previous example. Region 250, for instance, can be the hypodermis.

FIGS. 4A and 4B show another schematic representation of a system 208 for creating islets of treatment. FIGS. 4A and 4B show a system for delivering optical radiation to a treatment volume V located at a depth d in the patient's skin and having an area A. FIGS. 4A and 4B also show treatment or target portions 214 (i.e., islets of treatment) in the patient's skin 200. A portion of a patient's skin 200 is shown, which portion includes an epidermis 202 overlying a dermis 204, the junction of the epidermis and dermis being referred to as the dermis-epidermis (DE) junction 206. The treatment volume may be at the surface of the patient's skin (i.e., d=0) such that islets of treatment are formed in the stratum corneum. In addition, the treatment volume V may be below the skin surface in one or more skin layers or the treatment volume may extend from the skin surface through one or more skin layers.

The system 208 of FIGS. 4A and 4B can be incorporated within a hand held device. System 208 includes an energy source 210 to produce electromagnetic radiation (EMR). The output from energy source 210 is applied to an optical system 212, which is preferably in the form of a delivery head in contact with the surface of the patient's skin, as shown in FIG. 4B. The delivery head can include, for example, a contact plate or cooling element 216 that contacts the patient's skin. The system 208 can also include detectors 216 and controllers 218. The detectors 216 can, for instance, detect contact with the skin and/or the speed of movement of the device over the patient's skin and can, for example, image the patient's skin. The controller 218 can be used, for example, to control the pulsing of an EMR source in relation to contact with the skin and/or the speed of movement of the hand piece.

The image can be used to control the ablation process.

Additionally or alternatively, the image can employ “cross-hairs” or other mechanisms to more precisely focus the beams of EMR. For example, in one embodiment, the device is properly focused when the “cross-hairs” or other image is sharp, and can be fired—either manually or automatically. If all or a portion of the “cross-hairs” or other image are blurred and appear out of focus, the operator has a visual indication that the device is not properly focused or is at an improper distance or alignment relative to the tissue being treated. The operator would then know not to fire the device and/or the device could be designed to automatically prevent firing while providing the visual indication to the operator to aide in properly positioning the device.

Throughout this specification, the terms “head”, “hand piece” and “hand held device” may be used interchangeably.

D. Electromagnetic Radiation Sources

The energy source 210 may be any suitable optical energy source, including coherent and non-coherent sources, able to produce optical energy at a desired wavelength or a desired wavelength band or of multiple wavelengths or in multiple wavelength bands. The exact energy source 210, and the exact energy chosen, may be a function of the type of treatment to be performed, the tissue to be heated, the depth within the tissue at which treatment is desired, and of the absorption of that energy in the desired area to be treated. For example, energy source 210 may be a radiant lamp, a halogen lamp, an incandescent lamp, an arc lamp, a fluorescent lamp, a light emitting diode, a laser (including diode and fiber lasers), the sun, or other suitable optical energy source. In addition, multiple energy sources may be used which are identical or different. For example, multiple laser sources may be used and they may generate optical energy having the same wavelength or different wavelengths. As another example, multiple lamp sources may be used and they may be filtered to provide the same or different wavelength band or bands. In addition, different types of sources may be included in the same device, for example, mixing both lasers and lamps.

Energy source 210 may produce electromagnetic radiation, such as near infrared or visible light radiation over a broad spectrum, over a limited spectrum, or at a single wavelength, such as would be produced by a light emitting diode or a laser. In certain cases, a narrow spectral source may be preferable, as the wavelength(s) produced by the energy source may be targeted towards a specific tissue type or may be adapted for reaching a selected depth. In other embodiments, a wide spectral source may be preferable, for example, in systems where the wavelength(s) to be applied to the tissue may change, for example, by applying different filters, depending on the application. Acoustic, RF or other EMF sources may also be employed in suitable applications.

For example, UV, violet, blue, green, yellow light or infrared radiation (e.g., about 290-600 nm, 1400-3000 nm) can be used for treatment of superficial targets, such as vascular and pigment lesions, fine wrinkles, skin texture and pores. Blue, green, yellow, red and near IR light in a range of about 450 to about 1300 nm can be used for treatment of a target at depths up to about 1 millimeter below the skin. Near infrared light in a range of about 800 to about 1400 nm, about 1500 to about 1800 nm or in a range of about 2050 nm to about 2350 nm can be used for treatment of deeper targets (e.g., up to about 3 millimeters beneath the skin surface).

1. Coherent Optical Sources

Two particularly effective sources for the fractional ablation of tissue include an Er:YAG Laser operating at 2940 nm and an Er:YSGG Laser operating at 2780 nm.

Exemplary treatment parameters for Er:YAG and Er:YSGG laser sources are shown in Table B below.

TABLE B
Exemplary Parameters For Ablative Coherent Sources

| Parameter | Er: YAG Laser | Er: YSGG Laser |
|---|---|---|
| Wavelength | 2940 nm | 2780 nm |
| Pulsewidth | 0.25, 2 ms | 0.25, 2, 5, 10 ms |
| Diameter of Treatment Area | 5-9 mm | 5-9 mm |
| Optical Beam | Flat Top | Flat Top |
| Beam Diameter | 75-125 μm | 75-125 μm |
| Pitch | 200-1000 μm | 200-1000 μm |
| Beam Density | 800 beams/cm² | 800 beams/cm² |
| Beam Energy | Up to 10 mJ | Up to 10 mJ |
| | e.g., 7 mJ/1 ms, | e.g., 7 mJ/1 ms, |
| | 7 mJ/5 ms, | 7 mJ/5 ms, |
| | 7 mJ/10 ms, | 7 mJ/10 ms, |
| | 12 mJ/5 ms, and | 12 mJ/5 ms, and |
| | 12 mJ/10 ms | 12 mJ/10 ms |

Lasers and other coherent light sources can be used to cover wavelengths within the 100 to 100,000 nm range. This includes wavelengths that are in wavelength ranges typically used for non-ablative procedures such as 1320 μm, 1450 nm and 1540 nm. Examples of coherent energy sources are solid state, dye, fiber, and other types of lasers. For example, a solid state laser with lamp or diode pumping can be used. The wavelength generated by such a laser can be in the range of 400-3,500 nm. This range can be extended to 100-20,000 nm by using non-linear frequency converting. One such laser is a 3 μm Erbium laser. Solid state lasers can provide maximum flexibility with pulse width range from femtoseconds to a continuous wave, preferably in a range of approximately 1 femtosecond to 100 milliseconds. When very short pulses of EMR are used to create micro-islets, the wavelength has a smaller effect. For example, when a pulse on the order of several femtoseconds is applied, the relationship between the wavelength and the focal area is less pronounced such that longer wavelengths may be used to create small structures.

Another example of a coherent source is a tunable laser. For example, a dye laser with non-coherent or coherent pumping, which provide wavelength-tunable light emission. Dye lasers can utilize a dye dissolved either in liquid or solid matrices. Typical tunable wavelength bands cover 400-1,200 nm and a laser bandwidth of about 0.1-10 nm. Mixtures of different dyes can provide multi wavelength emission. Dye laser conversion efficiency is about 0.1-1% for non-coherent pumping and up to about 80% with coherent pumping. Laser emission could be delivered to the treatment site by an optical waveguide, or, in other embodiments, a plurality of waveguides or laser media could be pumped by a plurality of laser sources (lamps) next to the treatment site. Such dye lasers can result in energy exposure up to several hundreds of J/cm², pulse duration from picoseconds to tens of seconds, and a fill factor from about 0.1% to 90%.

Another example of a coherent source is a fiber laser. Fiber lasers are active waveguides a doped core or undoped core (Raman laser), with coherent or non-coherent pumping. Rare earth metal ions can be used as the doping material. The core and cladding materials can be quartz, glass or ceramic. The core diameter could be from microns to hundreds of microns. Pumping light could be launched into the core through the core facet or through cladding. The light conversion efficiency of such a fiber laser could be up to about 80% and the wavelength range can be from about 1,100 to 3,000 nm. A combination of different rare-earth ions, with or without additional Raman conversion, can provide simultaneous generation of different wavelengths, which could benefit treatment results. The range can be extended with the help of second harmonic generation (SHG) or optical parametric oscillator (OPO) optically connected to the fiber laser output. Fiber lasers can be combined into the bundle or can be used as a single fiber laser. The optical output can be directed to the target with the help of a variety of optical elements described below, or can be directly placed in contact with the skin with or without a protective/cooling interface window. Such fiber lasers can result in energy exposures of up to about several hundreds of J/cm² and pulse durations from about picoseconds to tens of seconds.

Diode lasers can be used for the 400-100,000 nm range. Since many photodermatology applications require a high-power light source, the configurations described below using diode laser bars can be based upon about 10-100 W, 1-cm-long, cw diode laser bar. Note that other sources (e.g., LEDs and microlasers) can be substituted in the configurations described for use with diode laser bars with suitable modifications to the optical and mechanical sub-systems.

Other types of lasers (e.g., gas, excimer, etc.) can also be used.

2. Non-Coherent Light Sources

A variety of non-coherent sources of electromagnetic radiation (e.g., arc lamps, incandescence lamps, halogen lamps, light bulbs) can be used for the energy source 210. There are several monochromatic lamps available such as, for example, hollow cathode lamps (HCL) and electrodeless discharge lamps (EDL). HCL and EDL could generate emission lines from chemical elements. For example, sodium emits bright yellow light at 550 nm. The output emission could be concentrated on the target with reflectors and concentrators. Energy exposures up to about several tens of J/cm², pulse durations from about picoseconds to tens of seconds, and fill factors of about 1% to 90% can be achieved.

Linear arc lamps use a plasma of noble gases overheated by pulsed electrical discharge as a light source. Commonly used gases are xenon, krypton and their mixtures, in different proportions. The filling pressure can be from about several torr to thousands of torr. The lamp envelope for the linear flash lamp can be made from fused silica, doped silica or glass, or sapphire. The emission bandwidth is about 180-2,500 nm for clear envelope, and could be modified with a proper choice of dopant ions inside the lamp envelope, dielectric coatings on the lamp envelope, absorptive filters, fluorescent converters, or a suitable combination of these approaches.

In some embodiments, a Xenon-filled linear flash lamp with a trapezoidal concentrator made from BK7 glass can be used. As set forth in some embodiments below, the distal end of the optical train can, for example, be an array of micro-prisms attached to the output face of the concentrator. The spectral range of EMR generated by such a lamp can be about 300-2000 nm, energy exposure can be up to about 1,000 J/cm², and the pulse duration can be from about 0.1 ms to 10 s.

Incandescent lamps are one of the most common light sources and have an emission band from 300 to 4,000 nm at a filament temperature of about 2,500 C. The output emission can be concentrated on the target with reflectors and/or concentrators. Incandescent lamps can achieve energy exposures of up to about several hundreds of J/cm² and pulse durations from about seconds to tens of seconds.

Halogen tungsten lamps utilize the halogen cycle to extend the lifetime of the lamp and permit it to operate at an elevated filament temperature (up to about 3,500 C), which greatly improves optical output. The emission band of such a lamp is in the range of about 300 to 3,000 nm. The output emission can be concentrated on the target with reflectors and/or concentrators. Such lamps can achieve energy exposures of up to thousand of J/cm² and pulse durations from about 0.2 seconds to continuous emission.

Light-emitting diodes (LEDs) that emit light in the 290-2,000 nm range can be used to cover wavelengths not directly accessible by diode lasers.

Referring again to FIGS. 4A and 4B, the energy source 210 or the optical system 212 can include any suitable filter able to select, or at least partially select, certain wavelengths or wavelength bands from energy source 210. In certain types of filters, the filter may block a specific set of wavelengths. It is also possible that undesired wavelengths in the energy from energy source 210 may be wavelength shifted in ways known in the art so as to enhance the energy available in the desired wavelength bands. Thus, filter may include elements designed to absorb, reflect or alter certain wavelengths of electromagnetic radiation. For example, filter may be used to remove certain types of wavelengths that are absorbed by surrounding tissues. For instance, dermis, hypodermis and epidermis tissues are primarily composed of water, as is much of the rest of the human body. By using a filter that selectively removes wavelengths that excite water molecules, the absorption of these wavelengths by the body may be greatly reduced, which may contribute to a reduction in the amount of heat generated by light absorption in these molecules. Thus, by passing radiation through a water-based filter, those frequencies of radiation that may excite water molecules will be absorbed in the water filter, and will not be transmitted into tissue. Thus, a water-based filter may be used to decrease the amount of radiation absorbed in tissue above the treatment region and converted into heat. For other treatments, absorption of the radiation by water may be desired or required for treatment.

E. Alternate Embodiments of Optical Systems

Generally, optical system 212 of FIGS. 4A and 4B functions to receive radiation from the source 210 and to focus/concentrate such radiation to one or more beams 222 directed to a selected one or more treatment or target portions 214 of volume V, the focus being both to the depth d and spatially in the area A (see FIG. 4B). Some embodiments use such an optical system 212, and other embodiments do not use an optical system 212. In some embodiments, the optical system 212 creates one or more beams which are not focused or divergent. In embodiments with multiple sources, optical system 212 may focus/concentrate the energy from each source into one or more beams and each such beam may include only the energy from one source or a combination of energy from multiple sources.

If an optical system 212 is used, the energy of the applied light can be concentrated to deliver more energy to target portions 214. Depending on system parameters, portions 214 may have various shapes and depths as described above.

The optical system 212 as shown in FIGS. 4A and 4B may focus energy on portions 214 or a selected subset of portions 214 simultaneously. Alternatively, the optical system 212 may contain an optical or mechanical-optical scanner for moving radiation focused to depth d to successive portions 214. In another alternative embodiment, the optical system 212 may generate an output focused to depth d and may be physically moved on the skin surface over volume V, either manually or by a suitable two-dimensional or three-dimensional (including depth) positioning mechanism, to direct radiation to desired successive portions 214. For the two later embodiments, the movement may be directly from portion to portion to be focused on or the movement may be in a standard predetermined pattern, for example a grid, spiral or other pattern, with the EMR source being fired only when over a desired portion 214.

Where an acoustic, RF or other non-optical EMR source is used as energy source 210, the optical system 212 can be a suitable system for concentrating or focusing such EMR, for example a phased array, and the term “optical system” should be interpreted, where appropriate, to include such a system.

While as may be seen from Table C, depth d for volume V and the focal depth of optical system 212 are substantially the same when focusing to shallow depths, it is generally necessary in a scattering medium such as skin to focus to a greater depth, sometimes a substantially greater depth, in order to achieve a focus at a deeper depth d. The reason for this is that scattering prevents a tight focus from being achieved and results in the minimum spot size, and thus maximum energy concentration, for the focused beam being at a depth substantially above that at which the beam is focused. The focus depth can be selected to achieve a minimum spot size at the desired depth d based on the known characteristics of the skin.

Both scattering and absorption are wavelength dependent. Therefore, while for shallow depths a fairly wide band of wavelengths can be utilized while still achieving a focused beam, the deeper the focus depth, the more scattering and absorption become factors, and the narrower the band of wavelengths available at which a reasonable focus can be achieved. Table C indicates preferred wavelength bands for various depths, although acceptable, but less than optimal, results may be possible outside these bands.

TABLE C

| Depth of | Wavelength range, | Numerical Aperture |
|---|---|---|
| damage, μm | nm | range |
| 0-200 | 290-10000 | <3 |
| 200-300 | 400-1880 & 2050- | <2 |
| | 2350 | |
| 300-500 | 600--1850 & 2150- | <2 |
| | 2260 | |
| 500-1000 | 600-1370 & 1600- | <1.5 |
| | 1820 | |
| 1000-2000 | 670-1350 & 1650- | <1 |
| | 1780 | |
| 2000-5000 | 800-1300 | <1 |

Numerical aperture is a function of the angle 9 for the focused radiation beam 222 from optical device 212. It is preferable that this number, and thus the angle 9, be as large as possible so that the energy at portions 214 in volume V where radiation is concentrated is substantially greater than that at other points in volume V (and in region 220), thereby minimizing damage to tissue in region 220, and in portions of volume V other than portions 214, while still achieving the desired therapeutic effect in the portions 214 of volume V. Higher numerical aperture of the beam increases safety of the epidermis, but it is limited by scattering and absorption of higher incidence angle optical rays. As can be seen from Table C above, the possible numerical aperture decreases as the focus depth increases.

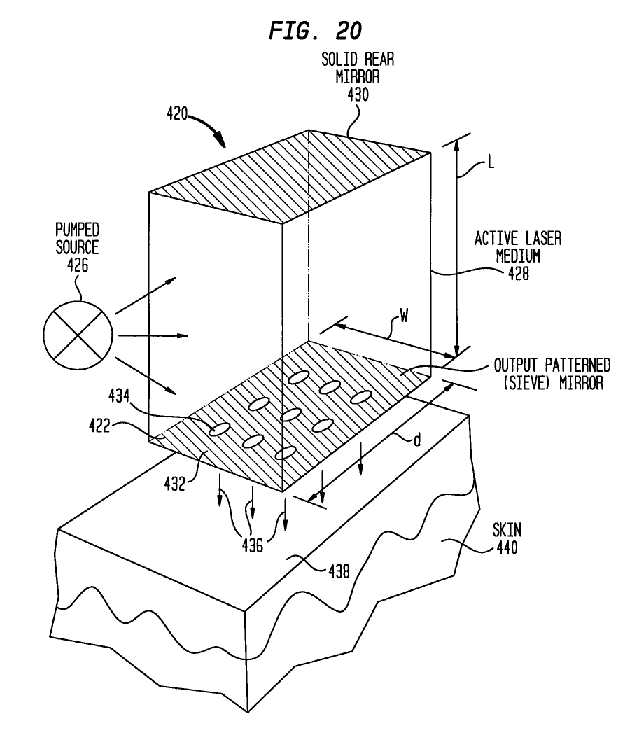

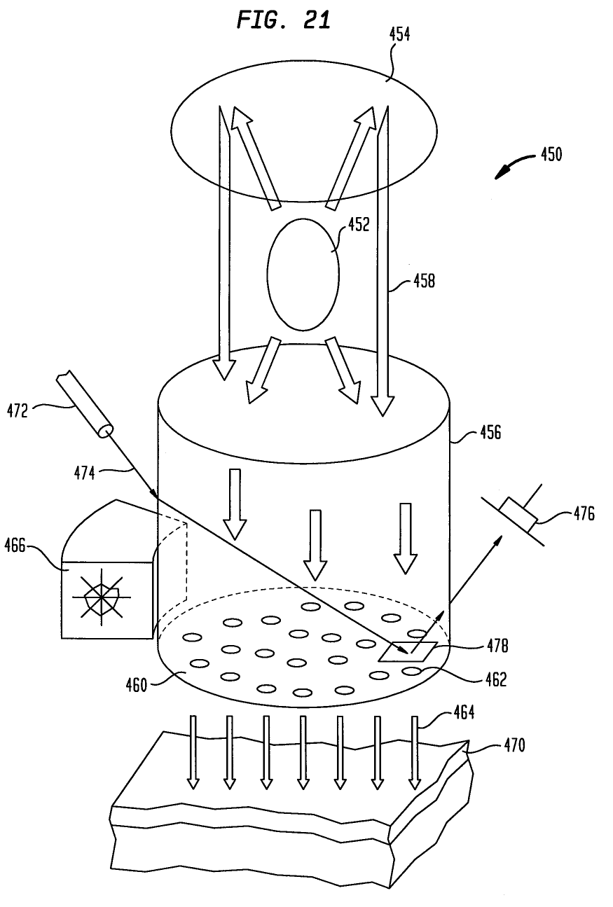

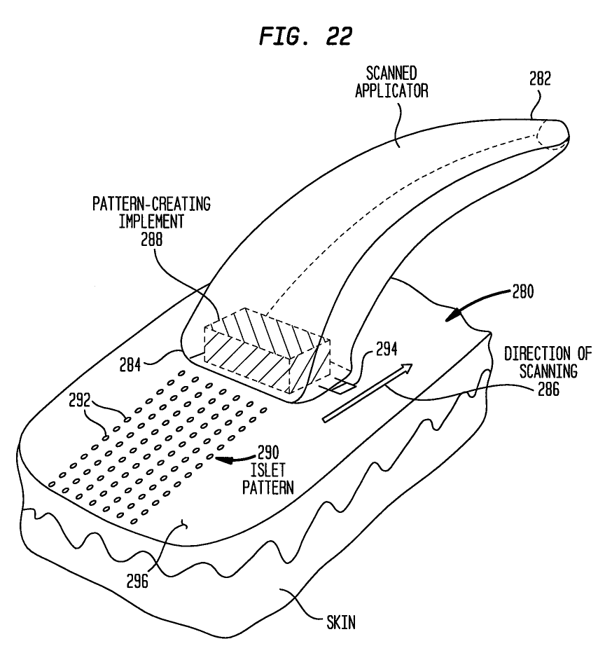

FIGS. 20 and 21 illustrate embodiments in which the islets of treatment are formed generally through the use of a mirror containing holes or other transmissive portions. Light passes through the holes in the mirror and strikes the patient's skin, creating islets of treatment. Light reflected by the mirror stays in the optical system and through a system of reflectors is re-reflected back toward the mirror which again allows light to pass through the holes. In this manner, the use of a mirror containing holes can be more efficient than the use of a mask with holes, where the mask absorbs rather than reflects light.

In the embodiment of FIG. 20, the patterned optical radiation to form the islets of treatment is generated by a specially designed laser system 420 and an output mirror 422. The laser system 420 and output mirror 422 can be contained in, for instance, a hand piece. In other embodiments, the laser system 420 can be contained in a base unit and the light passing through the holes in the mirror can be transported to the hand piece aperture through a coherent fiber optic cable. In still other embodiments, the laser can be mounted in the hand piece and beams from the laser can be directed to the skin with an optical system. In the illustrated embodiment, the laser system 420 comprises a pump source 426, which optically or electrically pumps the gain medium 428 or active laser medium. The gain medium 428 is in a laser cavity defined by rear mirror 430 and output mirror 422. Any type of EMR source, including coherent and non-coherent sources, can be used in this embodiment instead of the particular laser system 420 shown in FIG. 20.

According to one embodiment, the output mirror 422 includes highly reflective portions 432 that provide feedback (or reflection) into the laser cavity. The output mirror 422 also includes highly transmissive portions 434, which function to produce multiple beams of light that irradiate the surface 438 of the patient's skin 440. FIG. 20 depicts the highly transmissive portions 434 as being circular shapes, although other shapes, including, for example, rectangles, lines, or ovals, can also be used. The transmissive portions 434 can, in some embodiments, be holes in the mirror. In other examples, the transmissive portions 434 include partially transparent micro mirrors, uncoated openings, or openings in the mirror 422 with an antireflection coating. In these embodiments, the rest of the output mirror 422 is a solid mirror or an uncoated surface.

In one implementation, the output mirror 422 functions as a diffractive multi-spot sieve mirror. Such an output mirror 422 can also serve as a terminal or contact component of the optical system 420 to the surface 438 of the skin 440. In other embodiments, the output mirror 422 can be made from any reflective material.

Because of the higher refractive index of the illuminated tissue of the skin 440, divergence of the beams is reduced when it is coupled into the skin 440. In other embodiments, one or more optical elements (not shown) can be added to the mirror 422 in order to image the output of mirror 422 onto the surface of the skin 440. In this latter example, the output mirror 422 is usually held away from the skin surface 438 by a distance dictated by the imaging optical elements.

Proper choice of the laser cavity length L, operational wavelength λ of the source 426, the gain g of the laser media 428, dimensions or diameter D of the transmissive portions 434 (i.e., if circular) in the output mirror 422, and the output coupler (if used) can help to produce output beams 436 with optimal properties for creating islets of treatment. For example, when D2/4λL<1, effective output beam diameter is made considerably smaller than D, achieving a size close to the system's wavelength λ of operation. This regime can be used to produce any type of treatment islets.

Typically, the operational wavelength ranges from about 0.29 μm to 100 μm and the incident fluence is in the range from 1 mJ/cm² to 100 J/cm². The effective heating pulse width can be in the range of less than 100 times the thermal relaxation time of a patterned compound (e.g., from 100 fsec to 1 sec).

In other embodiments, the chromophore layer is not used. Instead the wavelength of light is selected to directly create the pathways.

In one example, the spectrum of the light is in the range of or around the absorption peaks for water. These include, for example, 970 nm, 1200 nm, 1470 nm, 1900 nm, 2940 nm, and/or any wavelength >1800 nm. In other examples, the spectrum is tuned close to the absorption peaks for lipids, such as 0.92 μm, 1.2 μm, 1.7 μm, and/or 2.3 μm, and wavelengths like 3.4 μm, and longer or absorption peaks for proteins, such as keratin, or other endogenous tissue chromophores contained in the SC.

The wavelength can also be selected from the range in which this absorption coefficient is higher than 1 cm⁻¹, such as higher than about 10 cm¹. Typically, the wavelength ranges from about 0.29 μm to 100 μm and the incident fluence is in the range from 1 mJ/cm² to 1000 J/cm². The effective heating pulse width is preferably less than 100× thermal relaxation time of the targeted chromophores (e.g., from 100 fsec to 1 sec).

The embodiment of FIG. 20 can be used to create islets of treatment in the stratum corneum. Controlling permeability of the stratum corneum can also be accomplished by absorption, scattering, or refractive coupling. Skin or topical cooling can be applied to prevent SC damage between the pathways and to control their size.

FIG. 21 depicts a second embodiment of a hand piece 450 that uses a mirror in order to reflect portions of EMR, while allowing certain patterns of the EMR to pass through holes in order to create islets of treatment. The embodiment of FIG. 21 includes a light source 452 and, in some embodiments, beam-shaping optics 454 and a waveguide 456. These components can be in a hand piece 450, such as those hand pieces set forth above. In other embodiments, the light source 452 can be in a base unit outside of the hand piece 450. The light source 452 can be a laser, a flashlamp, a halogen lamp, an LED, or another coherent or thermal source. In short, the light source 452 can be any type of EMR source as set forth above. The beam-shaping optics 454 can be reflective or refractive and can serve to direct EMR downward toward the output of the hand piece. The beam-shaping optics 454 can generally be disposed above and to the sides of the light source 452. The waveguide 456 can be used, for example, for homogenization of the beam 458.

The hand piece 150 of the embodiment of FIG. 21 can also include an output window 460 near the optical output from the hand piece 450. The output window 460 can be coated with a generally non-transparent coating. The coating can be, for instance, a reflective coating, such as a multi-layer dielectric coating. Such a dielectric coating can be selected to have a high reflectance over a spectral band defined by the EMR source 452. If selected to be highly reflective, such a dielectric coating will not absorb a large amount of light causing it to heat up. In addition, the window with the dielectric coating can be cooled if necessary for heat removal from the skin. Such a dielectric coating can be fabricated by vacuum deposition of one or, more likely, multiple dielectric layers. In some embodiments, the output window 460 can be made from a lattice of micro lenses that serves to provide spatial modulation of the power density in the lattice of optical islets.

The coating of the output window 460 can have a number of openings (or holes or transmissive portions) 462, which reshape the output beam into a plurality of beamlets 464. The openings 464 can be coated with anti-reflective coatings, or can contain Fresnel or refractive lenses for angular beam shaping. The openings 464 do not necessarily have to be of circular shape, as depicted in FIG. 21. The shape of the openings 464 can be adjusted depending on the skin condition to be treated. For example, the openings 464 can be circular, slits, rectangles, ovals, lines, or irregular shapes. In some embodiments, the shape of the openings 464 can be changed on demand (adaptively) depending on underlying skin conditions by using, for example, an electro-optical or thermo-optical effect.

The device can contain a cooling implement 466 to provide active contact cooling to the treatment area. The cooling implement 466 can be, for example, a sapphire cooling plate that is cooled by a water manifold or the like built into the hand piece, as set forth above. In addition, any other type of cooling implement 466, such as those set forth above, can be used.

The device of the embodiment of FIG. 21 can also include a device for monitoring the temperature of the waveguide 456 and/or the patient's skin 470. The temperature monitoring can be done, for example, using an optical device. In such an embodiment, a separate optical source 472 can be used to shine a probing beam 474 onto the output window 460. The reflected light is then detected with a detector 476. When the refractive indices of the layers in the multi-layer dielectric coating (or mirror or output window 460) change as a result of temperature change, the reflection coefficient of the coating changes as well. Thus, a temperature change can be deduced from the reflection measurements. A section 478 of the output window 460 can be optically separated from the skin 470 in order to reduce background parasitic signal from the skin 470 in measuring the temperature of the output window 460. The optical source 472 and the detector 476 can be built into the hand piece.

In some embodiments, the openings 462 in the output window 460 can be coated with phase-changing material, which changes its transparency as a result of temperature change. That is, the transparency of the openings 462 decreases when the temperature increases. Thus, overheating of skin 470 can be prevented by blocking the beamlets 474 with the decreased transparency of the openings 416.

In operation, the output window 460 is brought into contact with the treatment area 470 (i.e., the patient's skin). The light source 452 is then fired to output radiation from the hand piece. The openings 462 in the output window 462 form islets of treatment on the patient's skin 470.

The device of FIG. 21 can be used either in the stamping modes or the sliding modes. A stamping mode is a mode in which the device is placed on the skin and the radiation source is activated while the device remains stationary on the skin. In the sliding mode, the device can be moved over the skin while in contact with the skin. In the stamping modes, the resulting temperature in the skin (and, possibly, the damage profile) is completely determined by the geometry of the openings and the illumination/cooling parameters. In the sliding modes, an additional degree of control is available by varying the velocity of scanning.

The device of FIG. 21 can have an optical coating (i.e., on the treatment window 460) to provide light spatial modulation. Some embodiments can use technology similar to a gradient mirror, which is a mirror with variable transmission over its radius. An embodiment including a plurality of gradient mirrors could be beneficial for enhancement of parameters of the light source (such as the effect of photon recycling) and system cooling capabilities (very thin coating thickness).

In some embodiment, the coating, (such as, for example, a multilayer dielectric high reflective coating with lattice of transparent zones) can be coated directly on the contact cooling surface of a sapphire chilled bock. In such an embodiment, the entire sapphire block can be used as a cooling area, but the irradiated area is limited by the area of the transparent zones. Such an embodiment can be effective for a combination of LOI treatment with skin upper layer protection for deep dermal or fat treatments.

In another embodiment, where a laser source is used, the laser itself can have an output that is not uniform. For example, in such an embodiment, the laser itself can be surrounded by a reflector, which can be a high reflector. The reflector surrounding the laser, and in particular at the output end of the laser, can have areas that are less reflective than other areas. That is, the reflector in such an embodiment does not have uniform reflectivity. These areas can result in increased radiation output from the laser source in discrete areas (or holes). Thus, the reflector or mirror surrounding the laser can itself generated spatially modulated light as an output. The laser source can therefore be housed in a hand piece that has the laser source output close to the output from the hand piece. The hand piece can therefore be brought into close proximity to the skin and fired to create treatment islets.

F. Cooling Elements

As set forth above, the system 208 can also include a cooling element 215 to cool the surface of the skin 200 over treatment volume V. As shown in FIGS. 4A and 4B, a cooling element 215 can act on the optical system 212 to cool the portion of this system in contact with the patient's skin, and thus the portion of the patient's skin in contact with such element. In some embodiments intended for use on the stratum corneum, the cooling element 215 might not be used or, alternatively, might not be cooled during treatment (e.g., cooling only applied before and/or after treatment). In some embodiments, cooling can be applied fractionally on a portion of the skin surface (cooling islets), for example, between optical islets. In some embodiments, cooling of the skin is not required and a cooling element might not be present on the hand piece. In other embodiments, cooling may be applied only to the portions of tissue between the treatment islets in order to increase contrast.

The cooling element 215 can include a system for cooling the optical system (and hence the portion in contact with the skin) as well as a contact plate that touches the patient's skin when in use. The contact plate can be, for example, a flat plate, a series of conducting pipes, a sheathing blanket, or a series of channels for the passage of air, water, oil or other fluids or gases. Mixtures of these substances may also be used, such as a mixture of water and methanol. For example, in one embodiment, the cooling system can be a water-cooled contact plate. In another embodiment, the cooling mechanism may be a series of channels carrying a coolant fluid or a refrigerant fluid (for example, a cryogen), which channels are in contact with the patient's skin 200 or with a plate of the apparatus 208 that is in contact with the patient's skin. In yet another embodiment, the cooling system may comprise a water or refrigerant fluid (for example R134A) spray, a cool air spray or air flow across the surface of the patient's skin 200. In other embodiments, cooling may be accomplished through chemical reactions (for example, endothermic reactions), or through electronic cooling, such as Peltier cooling. In yet other embodiments, cooling mechanism 215 may have more than one type of coolant, or cooling mechanism 215 and/or contact plate may be absent, for example, in embodiments where the tissue is cooled passively or directly, for example, through a cryogenic or other suitable spray. Sensors or other monitoring devices may also be embedded in cooling mechanism 215 or other portions of the hand held device, for example, to monitor the temperature, or determine the degree of cooling required by the patient's skin 200, and may be manually or electronically controlled.

In certain cases, cooling mechanism 215 may be used to maintain the surface temperature of the patient's skin 200 at its normal temperature, which may be, for example, 37 or 32° C., depending on the type of tissue being heated. In other embodiments, cooling mechanism 215 may be used to decrease the temperature of the surface of the patient's skin 200 to a temperature below the normal temperature of that type of tissue. For example, cooling mechanism 215 may be able to decrease the surface temperature of tissue to, for example, a range between 25° C. and −5° C. In other embodiments, a plate can function as a heating plate in order to heat the patient's skin. Some embodiments can include a plate that can be used for cooling and heating.

A contact plate of the cooling element 215 may be made out of a suitable heat transfer material, and also, where the plate contacts the patient's skin 200, of a material having a good optical match with the tissue. Sapphire is an example of a suitable material for the contact plate. Where the contact plate has a high degree of thermal conductivity, it may allow cooling of the surface of the tissue by cooling mechanism 215. In other embodiments, contact plate may be an integral part of cooling mechanism 215, or may be absent. In some embodiments, such as shown in FIGS. 4A and 4B, energy from energy source 210 may pass through contact plate. In these configurations, contact plate may be constructed out of materials able to transmit at least a portion of energy, for example, glass, sapphire, or a clear plastic. In addition, the contact plate may be constructed in such a way as to allow only a portion of energy to pass through contact plate, for example, via a series of holes, passages, apertures in a mask, lenses, etc. within the contact plate. In other embodiments, energy may not be directed through the cooling mechanism 215.

In certain embodiments, various components of system 208 may require cooling. For example, in the embodiment shown in FIGS. 4A and 4B, energy source 210, optics 212, and filter may be cooled by a cooling mechanism (not shown). The design of cooling mechanism may be a function of the components used in the construction of the apparatus. The cooling element 215 for the patient's skin 200 and the cooling element for the components of the system 208 may be part of the same system, separate systems or one or both may be absent. Cooling mechanism for the components of the system 208 may be any suitable cooling mechanism known in the art. Cooling of the components may be accomplished through convective or conductive cooling. In some embodiments, the cooling element can prevent optics 212 from overheating due absorption of EMR.

Typically cooler 215 is activated before source 210 to pre-cool the patient's skin to a selected temperature below normal skin temperature, for example −5° C. to 10° C., to a depth of at least DE junction 206, and preferably to depth d to protect the entire skin region 220 above volume V. However, if pre-cooling extends for a period sufficient for the patient's skin to be cooled to a depth below the volume V, and in particular if cooling continues after the application of radiation begins, then heating will occur only in the radiated portions 214, each of which portions will be surrounded by cooled skin. Therefore, even if the duration of the applied radiation exceeds TRT for portions 214, heat from these portions will be contained and thermal damage will not occur beyond these portions. Further, while nerves may be stimulated in portions 214, the cooling of these nerves outside of portions 214 will, in addition to permitting tight control of damage volume, also block pain signals from being transmitted to the brain, thus permitting treatments to be effected with greater patient comfort, and in particular permitting radiation doses to be applied to effect a desired treatment which might not otherwise be possible because of the resulting pain experienced by the patient.

G. Other Devices for Producing a Multiplicity of Treated Islets

A number of different devices and structures can be used to spatially modulate and/or concentrate EMR in order to generate islets of treatment in the skin. For example, the devices can use reflection, refraction, interference, diffraction, and deflection of incident light to create treatment islets. A detailed explanation of such are provided in the related applications listed above that have been incorporated by reference in their entirety.

In other embodiments, spatially selective islets of treatment can be created by applying to the skin surface a desired pattern of a topical composition containing a preferentially absorbing exogenous chromophore. The chromophore can also be introduced into the tissue with a needle, for example, a micro needle as used for tattoos. In this case, the EMR energy may illuminate the entire skin surface where such pattern of topical composition has been applied. Upon application of appropriate EMR, the chromophores can heat up, thus creating islets of treatment in the skin. Alternatively, the EMR energy may be focused on the pattern of topical composition. A variety of substances can be used as chromophores including, but not limited to, carbon, metals (Au, Ag, Fe, etc.), organic dyes (Methylene Blue, Toluidine Blue, etc.), non-organic pigments, nanoparticles (such as fullerenes), nanoparticles with a shell, carbon fibers, etc. The desired pattern can be random and need not be regular or pre-determined. It can vary as a function of the skin condition at the desired treatment area and be generated ad hoc.

Some embodiments provide a film or substrate material with a lattice of dots, lines or other shapes, either on the surface of the film or embedded within the film, in which the dots, lines or other shapes include a chromophore appropriate to the EMR source. The dots, lines or other shapes may be the same or different sizes and different shapes may be included on the film.

The dots, lines or other shapes may be formed from a material that can be glued, welded or otherwise attached to the stratum corneum to create islets, and such attachment may be sufficient to allow the film to be removed from the skin while leaving the dots, lines or other shapes on the skin. For example, the dots, lines or other shapes may be formed of an ultraviolet curing compound such that when the film is applied to the skin and ultraviolet light is applied to the film, the dots, lines or other shapes are attached to the skin and the film may be removed prior to EMR energy being applied. In other cases, the dots, lines or other shapes may be formed of a suitable phase-changing material (e.g., albumin), which can be used for welding. In other cases, the film is not removed and the EMR energy is applied through the film.

In other methods, the dots, lines or other shapes may be manually applied to the skin individually or by spraying or other techniques. In other embodiments, the hand piece may apply the shapes to the skin prior to applying the EMR energy. As one example, the shapes may be contained in a lotion, gel, powder or other topical composition that is applied to the skin manually prior to using the hand piece to apply the EMR energy. Alternatively, the lotion is dispensed by the hand piece onto the skin prior to the hand piece delivering EMR energy. As another example, a film containing the shapes may be applied to the skin manually or by the hand held device (as for example a tape dispenser).

H. Controllers and Feedback Systems

Some embodiments can also include speed sensors, contact sensors, imaging arrays, and controllers to aid in various functions of applying EMR to the patient's skin. System 208 of FIG. 4A includes an optional detector 216, which may be, for example, a capacitive imaging array, a CCD camera, a photodetector, or other suitable detector for a selected characteristic of the patient's skin. The output from detector 216 can be applied to a controller 218, which is typically a suitably programmed microprocessor or other such circuitry, but may be special purpose hardware or a hybrid of hardware and software. Control 218 can, for example, control the turning on and turning off of the light source 210 or other mechanism for exposing the light to the skin (e.g., shutter), and control 218 may also control the power profile of the radiation. Controller 218 can also be used, for example, to control the focus depth for the optical system 212 and to control the portion or portions 214 to which radiation is focused/concentrated at any given time. Finally, controller 218 can be used to control the cooling element 215 to control both the skin temperature above the volume V and the cooling duration, both for pre-cooling and during irradiation.

I. Creation of Lattices Using Non-Optical EMR Sources

The lattices can also be produced using non-optical sources. For example, ultrasound, microwave, radio frequency and low frequency or DC EMR sources can be used as energy sources to create lattices of EMR-treated islets. In addition, for treating tissue surfaces, the tissue surface can be directly contacted with heating elements in the pattern of the desired lattice. Also, various optical and/or non-optical sources can be combined, such as visible light, acoustic energy, ultrasound, and shockwaves (e.g., formed by the application or heat, acoustic energy, ultrasound or other forms of energy). In addition, the sources can be combined with various mechanical stimuli, such as a vacuum or vibrating mechanism, to improve and facilitate the treatment of tissue.

J. Motion Sensors And Scanning Devices

A number of different devices and structures can be used to generate islets of treatment in the skin. FIG. 22 illustrates one system for producing the islets of treatment on the skin 280. An applicator 282 is provided with a handle so that its head 284 can be near or in contact with the skin 280 and scanned in a direction 286 over the skin 280. The applicator 282 can include an islet pattern generator 288 that produces a pattern of areas of enhanced permeability in the SC or arrangement 290 of islets particles 292 on the surface of the skin 280, which when treated with EMR from applicator 210 produces a pattern of enhanced permeability. In other embodiments, the generator 288 produces thermal, damage or photochemical islets into the epidermis or dermis.

In one embodiment, the applicator 282 includes a motion detector 294 that detects the scanning of the head 284 relative to the skin surface 296. This generated information is used by the islet pattern generator 288 to ensure that the desired fill factor or islet density and power is produced on the skin surface 296. For example, if the head 284 is scanned more quickly, the pattern generator responds by imprinting islets more quickly. The following description describes this embodiment, as well as other embodiments, in greater detail. Further, the following sections elaborate on the types of EMR sources that can be used with the applicator 282 and on the methods and structures that can be used to generate the islets of treatment.

According to one embodiment, an apparatus can include a light emitting assembly for applying optical energy to the target area of the patient's skin, a sensor for determining the speed of movement of the head portion across the target area of the patient's skin, and circuitry in communication with the sensor for controlling the optical energy in order to create islets of treatment. The circuitry can control, for example, pulsing of the optical energy source based on the speed of movement of the head portion across the skin in order to create islets of treatment. In another embodiment, the circuitry can control movement of the energy source within the apparatus based on the speed of movement of the head portion across the skin in order to treat certain areas of the skin, while not exposing other areas, in order to create islets of treatment.

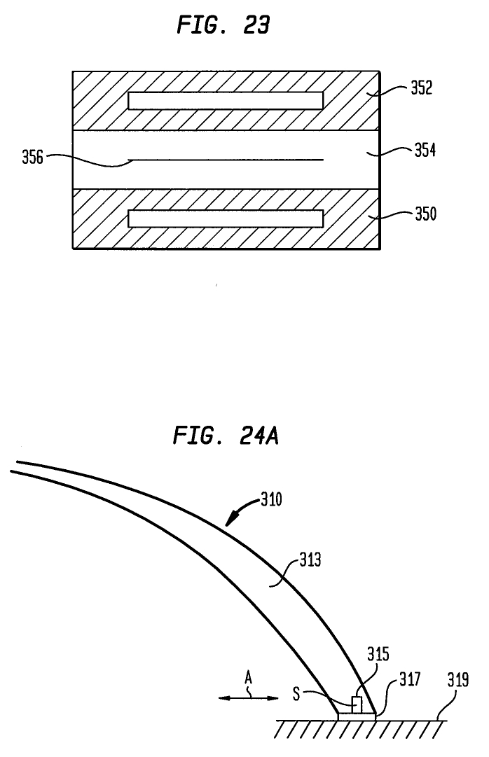

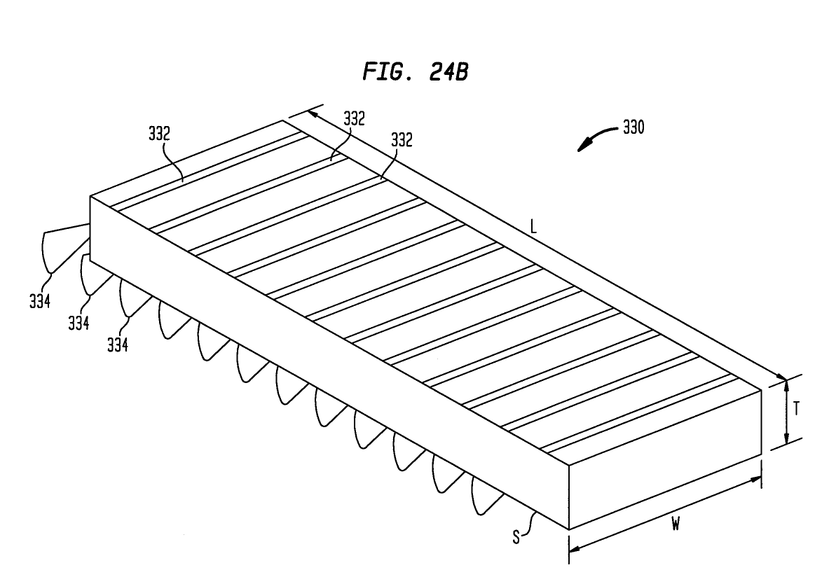

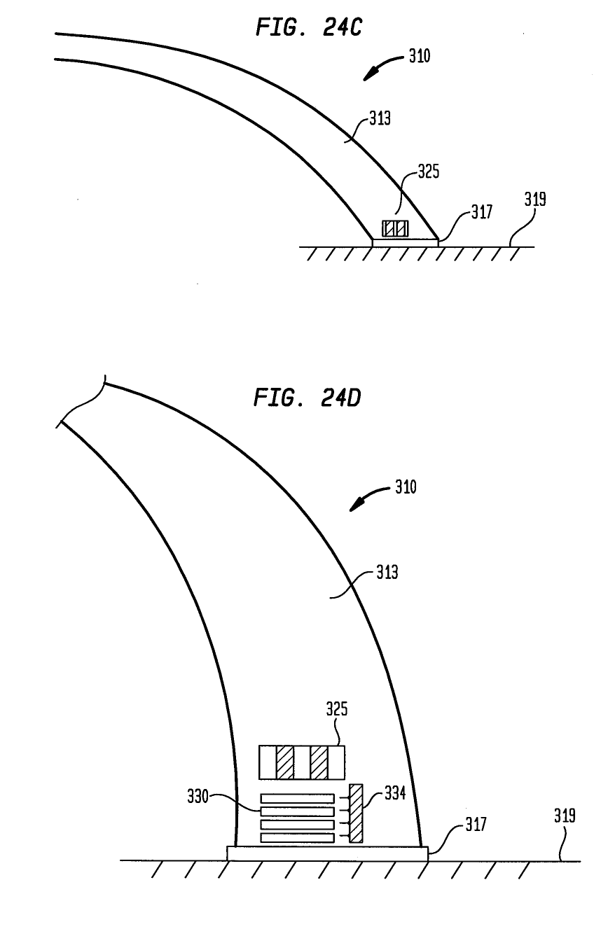

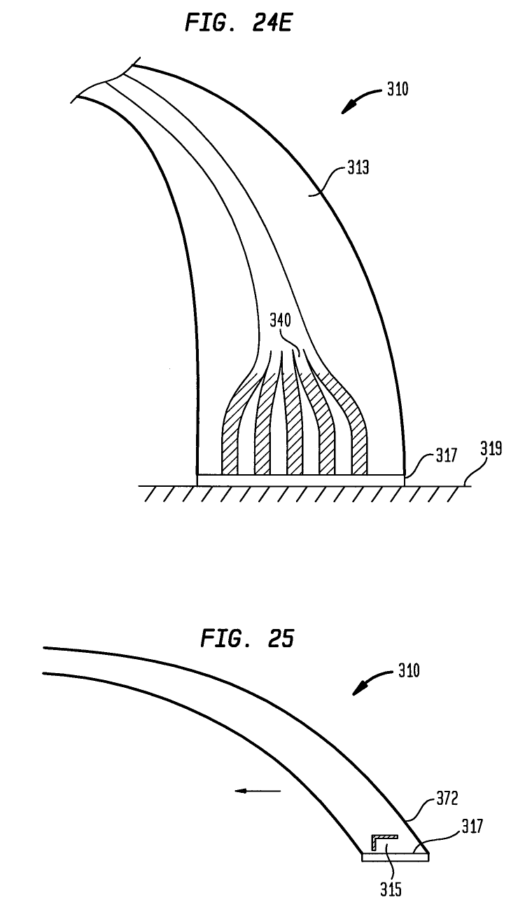

FIG. 23 is a bottom view of an embodiment that includes a speed sensor for measuring the speed of movement of the hand piece across the patient's skin. The embodiment of FIG. 23 can be used, for example, in the embodiment of FIG. 24A. That is, the hand piece 310 of FIG. 24A can include a housing 310, a diode laser bar 315 (or more than one diode laser bars as in FIG. 24C), and a plate 317. FIG. 23 shows a bottom view of a hand piece in which it is equipped with a speed sensor 350, 352.

A number of types of speed sensors can be used to measure the hand piece speed relative to the skin surface. For example, the speed sensor can be an optical mouse, a laser mouse, a wheel/optical encoder, or a capacitive imaging array combined with a flow algorithm similar to the one used in an optical mouse. A capacitive imaging array can be used to measure both hand piece speed and to create an image of the treated area. Capacitive imaging arrays are typically used for thumbprint authentication for security purposes. However, a capacitive imaging array can also be used to measure the hand piece speed across the skin surface. By acquiring capacitive images of the skin surface at a relatively high frame rate (for example, 100-2000 frames per second), a flow algorithm can be used to track the motion of certain features within the image and calculate speed.

In the embodiment of FIG. 23, two capacitive imaging arrays 350, 352 are located on the bottom of the hand piece, with one on each side of the treatment window 354. The diode laser bar 356 output is directed through the treatment window, that is, through a cooling plate or the like. The orientation of the capacitive imaging arrays 350, 352 can vary in different embodiments. As the device is moved, both capacitive imaging arrays 350, 352 measure the speed of the hand piece across the patient's skin. The configuration can include circuitry that is in communication with the capacitive imaging arrays 350, 352 to measure the speed and determine an appropriate rate for firing the light source (e.g., diode laser) based on that speed. The circuitry, therefore, can also be in communication with the laser in order to pulse the laser at an appropriate speed. The speed sensor incorporated in the hand piece, therefore, can provide feedback to the laser pulse generator. In some embodiments, after an initial pulse of radiation, the pulsing of the diode laser bar 356 might not be enabled until the capacitive imaging arrays 350, 352 sense movement of the hand piece over the skin. This circuitry can be located in the hand piece in some embodiments or, in other embodiments, in a base unit. When the diode laser bar 356 is enabled for firing by the user (for example by depressing a footswitch), a laser pulse generator for the laser fires the laser at a rate proportional to the hand piece speed.

In operation, the embodiment described above can be used to create a uniform matrix of treatment islets by manually moving a hand piece that includes a single diode laser bar (or multiple diode laser bars) across the skin surface and pulsing the laser at a rate proportional to the hand piece speed. For example, decreasing the time interval between laser pulses as the hand piece speed increases can be used to keep a constant matrix of lines of islets of treatment on the skin. Similarly, increasing the time interval between laser pulses as the hand piece speed decreases can be used to keep a constant matrix of lines of islets of treatment on the skin. The treatment head, including treatment window or light aperture of the hand piece, can be rotated to vary the spacing between islets of treatment in the direction orthogonal to hand piece movement.

In addition to measuring hand piece speed, the capacitive imaging arrays 350, 352 can also image the skin after the line of islets of treatment has been created in order to view the treatment results. Acquired images can be viewed in real time during treatment. The hand piece can include, for example, a display that shows the treatment area of the skin under the cooling plate. Alternatively, the acquired images can be stored in a computer for viewing after the treatment is complete. In some embodiments, the system can be configured to display images from both sensors, so that the hand piece can be moved either forward or backward.

In the configurations discussed above, the diode laser is used at a relatively low duty cycle because the laser is turned off in between islets of treatment. In some embodiments, the diode laser can be used more efficiently by keeping the diode laser on for a longer time, for example, if the of islets of treatment are lines instead of spots. FIG. 25 depicts an example of a hand piece 310 in which the diode laser bar 315 can be mounted on a miniature linear translator 372 inside the hand piece. The hand piece 310 of FIG. 25 can be largely the same as the embodiments set forth above. That is, it can include a diode laser bar 315 adjacent a plate 317 in a hand piece. This embodiment, however, also include a miniature linear translator 372 that can move the diode laser bar 315 in the forward or backward direction within the hand piece 310. Other suitable motors, such as, for example, a piezoelectric motor or any type of linear motor, can be used instead of the miniature linear translator 372. In alternative embodiments, the diode laser bar 315 can be mounted on a cylindrical shaft that can be rotated to accomplish the same function as the linear translator 372. A single-axis galvanometer-driven mirror can also be used.

In the embodiment of FIG. 25, as the hand piece 310 is moved forward (left in the Figure), the diode laser bar 315 would be moved backward (right in the Figure) within the hand piece at the same speed. After the diode laser bar 315 reaches the rear of the hand piece 310, it would be moved to the front of the hand piece, and the cycle would be repeated. The spacing between the lines of islets of treatment can be adjusted by varying the time required to move from the rear to the front of the hand piece 310. In this embodiment, for example, a speed sensor can measure the speed of movement of the hand piece 310 across the skin. This speed sensor can be similar to those described above. Such a speed sensor can be in communication with circuitry that moves the diode laser bar 315 (through the motor 372) based on the speed of the hand piece 310 across the skin. Thus, by appropriately moving the diode laser bar 315 within the hand piece 310, a matrix of treatment islets can be created on the patient's skin.

Another embodiment could include a speed sensor. In this embodiment, the hand piece is a non-coherent EMR source disposed within the housing of the hand piece. The non-coherent EMR source can be any of the types set forth above, including, for example, a linear flash lamp, an arc lamp, an incandescence lamp, or a halogen lamp. In one embodiment, the light source is a Xe-filled linear flash lamp. The hand piece can also include an optical reflector, one or more optical filters, and a light duct or concentrator. The optical reflector can serve to reflect and direct the light into the concentrator. The concentrator can be made from glass BK7, and can have a trapezoidal shape. In other embodiments, the concentrator can be made from different materials and its shape can vary. The concentrator can be used, for example, for homogenization of the beam. In some embodiments, the optical filter might not be used. If used, the filter can serve to filter out certain wavelengths of light from the EMR source. In addition, the optical reflector might not be used in some embodiments. In some embodiments, a cooling plate can be attached to the housing or at the end of the optical path in order to cool the patient's skin.

The housing can be equipped with a speed sensor. This speed sensor can measure the speed of movement of the housing with respect to the patient's skin. In the embodiment of, the housing of the hand piece is capable of movement independently from the light source within the housing. That is, when the housing moves with a speed V with respect to the patient's skin, the light source can move within the housing such that the light source remains fixed with respect to the patient's skin. That is, the speed v of the light source with respect to the patient's skin is approximately zero, which means that the light source would move relative to the housing and within the housing at a speed of −V. In this embodiment, the light source does not move and is held steady during application of radiation in order to guarantee the desired energy exposure. When treatment of the selected part of skin has been completed, the light source can move within the housing in order to reach its initial position. That is, the light source can move forward in a leap-frog manner with a speed v>V (where both v and V are measured relative to the patient's skin) for treatment of the next part of skin.

As set forth above, for synchronization of the speed V of the housing and the speed v of the light source, the housing is equipped with the speed sensor. The speed sensor can measure the movement of the housing with respect to the patient's skin and then move the light source within the housing at an appropriate speed in order to remain fixed with respect to the patient's skin. The hand piece or a base unit associated with the hand piece can include circuitry that receives the speed of movement of the housing and then sends a signal to a motor that moves the light source 404 within the housing 402 at an appropriate speed. The hand piece, therefore, can include a linear motor or linear translator, such as those set forth above, to move the light source within the housing.

The description above indicates that the light source 404 is moveable within the housing The reflector, the filter, and the concentrator, if used, can be connected to the light source in some embodiments in a manner so that these components move within the housing 402 along with the light source.

In some embodiments using a Xe-filled linear flash lamp, the spectral range of the EMR is 300-3000 nm, the energy exposure up to 1000 J/cm², the pulse duration is from about 0.1 ms to 10 s, and the fill factor is about 1% to 90%.

Another embodiment involves the use of imaging optics to image the patient's skin and use that information to determine medication application rates, application of EMR, or the like in order to optimize performance. For instance, some medical or cosmetic skin treatments require that the medication application rate be accurately measured and its effect be analyzed in real time. The skin surface imaging system can detect the size of reversible or irreversible holes created with techniques proposed in this specification for creating treatment islets in the stratum corneum. For this purpose, a capacitive imaging array can be used in combination with an image enhancing lotion and a specially optimized navigation/image processing algorithm to measure and control the application rate.

The use of a capacitive imaging array is set forth above in connection with FIG. 23. Such capacitive image arrays can be used, for example, within the applicator 282 of FIG. 22 according to this embodiment. As set forth above, in addition to measuring hand piece speed, the capacitive imaging arrays 350, 352 (FIG. 23) can also image the skin. Acquired images can be viewed in real time during treatment via a display window of the device.

One example of a suitable capacitive sensor for this embodiment is a sensor having an array of 8 image-sensing rows by 212 image-sensing columns. Due to inherent limitations of capacitive array technology, a typical capacitive array sensor is capable of processing about 2000 images per second. To allow for processing skin images in real time, an orientation of the sensor can be selected to aid in functionality. In one embodiment, for instance, the images are acquired and processed along the columns. This allows for accurate measurement of velocity up to about 200 mm/s.

For the sensor to function reliably and accurately, the skin surface can be treated with an appropriate lotion. In some embodiments, a properly selected lotion can improve the light-based skin treatment and navigation sensor operation. A lotion may be optically transparent to the selected wavelength, provide image enhancement to a sensor, and function as a friction reduction lubricant.

Circuitry containing a processing algorithm or the like can be in communication with the capacitive image sensor. The capacitive sensor and its associated processing algorithm are capable of determining a type of lotion and its effect on the skin surface. This can be performed in real time by sequentially analyzing the image spectral characteristics. The processing algorithm can also perform sensor calibration, image contrast enhancement, and filtering, as well as processing and control of images of the skin surface with navigation code to aid in various applications.

Real time acquired images can be used for statistical analysis of a marker concentration in a lotion. The markers are put in a lotion to function as identifiers of a treatment area. The marker can be a chromophore itself (i.e., a chromophore that heats up upon application of irradiation) or it can be a chemical that indicates the presence of the chromophore or medication in the lotion. As one example, the marker emits or reflects light proportional to the incident light to indicate the concentration of a chromophore or medication in the lotion. The capacitive sensor, therefore, can function to determine whether the marker concentration of a given lotion is at an appropriate level. The circuitry can, for instance, send a signal to the user of the concentration of the marker. Alternatively, the circuitry can determine if the marker concentration meets a preselected set point concentration level for a certain marker. If the set point is not met, the circuitry can communication to the user to let the user know that more (or perhaps less) lotion may be needed on the patient's skin. Selected markers with the right lotion pH level can also be used as an eye safety enhancement feature for light treatment on human body.

The sensor can also function as a contact sensor. This allows for real time determination of immediate contact of a hand piece with the patient's skin. The combination of hardware and software allows this determination within one image frame. The algorithm measures in real time a skin contact and navigation parameters (position, velocity and acceleration) along the x-axis and y-axis. This enhances the safety of light treatment on human skin by allowing for the control of the velocity and the quality of skin contact. The quality of contact can be a function of lotion type and pressure applied to the treatment device.

The capacitive sensor along with image processing and special lotion can be used for detecting a skin imperfection and measuring its size in real time. The resolution of the sensor will depend on pixel size, image processing and the sub pixel sampling.

The capacitive sensor and image processing allow for determination of whether the device is operating on biological skin or some form of other surface. It is possible under proper sampling conditions to extract the type of skin the device is moving across. This is accomplished by comparing real time processed images to a stored pattern or calculated set of parameters. In addition, the combination of the capacitive sensor and image pattern recognition, navigation algorithm, and special lotion, can be used to determine the presence of skin hair and provide statistical information about the density and size of the hair.

The capacitive sensor with a combination of two types of lotion, a calibrated skin penetration lotion and image enhancing lotion, can determine the effect of skin rejuvenation on skin over a large area. This analysis can be performed in real time by treating the skin with two lotions and then moving the capacitive sensor over the skin area of interest. The real time algorithm determines the effective area of treatment and the enhancement factor above the norm.

K. Hand piece with diode laser bar

Some embodiments use one or more diode laser bars as the EMR source. Because many photodermatology applications require a high-power light source, a standard 40-W, 1-cm-long, cw diode laser bar can be used in some embodiments. Any suitable diode laser bar can be used including, for example, 10-100 W diode laser bars. A number of types of diode lasers, such as those set forth above, can be used. Other sources (e.g., LEDs and diode lasers with SHG) can be substituted for the diode laser bar with suitable modifications to the optical and mechanical sub-systems.

FIG. 24A shows one embodiment using a diode laser bar. Many other embodiments can be used within the scope. In this embodiment, the hand piece 310 includes a housing 313, a diode laser bar 315, and a cooling or heating plate 317. The housing 313 supports the diode laser bar 315 and the cooling or heating plate 317, and the housing 313 can also support control features (not shown), such as a button to fire the diode laser bar 315. The housing 313 can be made from any suitable material, including, for example, plastics. The cooling plate, if used, can remove heat from the patient's skin. The heating plate, if used, can heat the patient's skin. The same plate can be used for heating or cooling, depending on whether a heat source or source of cooling is applied to the plate.

The diode laser bar 315 can be, in one embodiment, ten to fifty emitters (having widths of 50-to-150 μm in some embodiments or 100-to-150 μm in others) that are located along a 1-cm long diode bar with spacing of 50 to 900 μm. In other embodiments, greater than or less than fifty emitters can be located on the diode laser bar 315, the emitter spacing, and the length of the diode laser bar 315 can also vary. In addition, the width of the emitters can vary. The emitter spacing and the number of emitters can be customized during the manufacturing process.

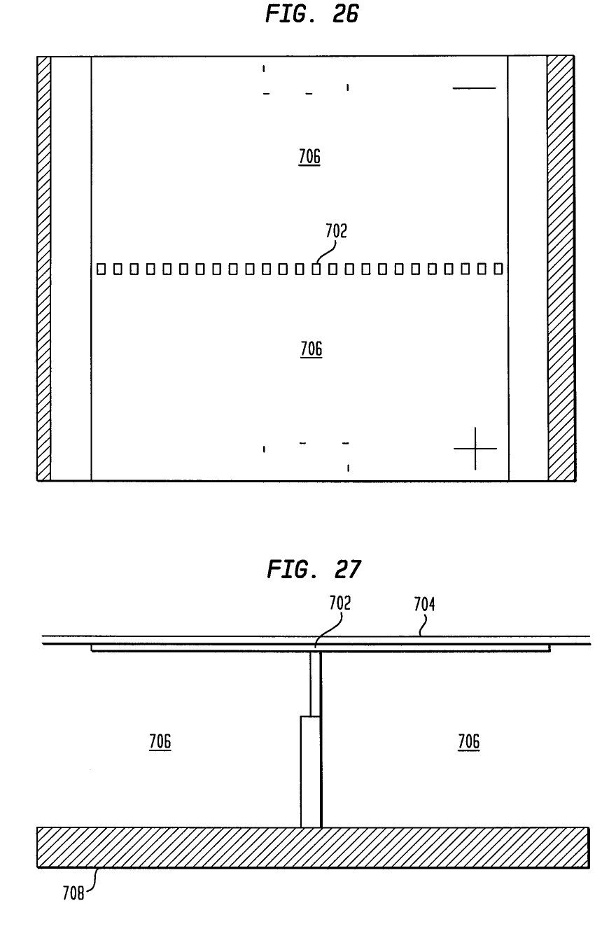

The diode laser bar 315 can be, in one embodiment, twenty-five 100-to-150 μm or 50-to-150 μm wide emitters that are located along a 1 cm long diode bar, each separated by around 50 to 900 microns in some embodiments, and approximately 500 microns in others. FIGS. 26 and 27 depict top and cross-sectional views, respectively, of such a diode laser bar assembly in this embodiment. In this embodiment, twenty-five emitters 702 are located directly beneath the surface plate 704 that is placed in contact with the skin during treatment. Two electrodes 706 are located to each side of the emitters 702. The bottom of the diode assembly contains a cooling agent 708 to control the diode laser and plate 704 temperatures.

In the embodiment of FIGS. 26 and 27, the divergence of the beam emanating from the emitters 702 is between 6 and 12 degrees along one axis (the slow axis) and between 60 and 90 degrees along the fast axis. The plate 704 may serve as either a cooling or a heating surface and also serves to locate the emitters 702 in close and fixed proximity to the surface of the tissue to be treated. The distance between the emitters 702 and the plate 704 can be between about 50 and 1000 micrometers, and more particularly between about 100 and 1000 micrometers in some embodiments, in order to minimize or prevent distortion effects on the laser beam without using any optics for low cost and simplicity of manufacture. During use, the distance between the emitters 702 and the patient's skin can be between about 50 and 1000 micrometers, and more particularly 100 and 1000 micrometers in some embodiments. In such embodiments, imaging optics are not needed, but other embodiments could include additional optics to image the emitter surfaces 702 directly onto the tissue surface. In other embodiments, greater than or less than twenty-five emitters can be located on the diode laser bar, and the length of the diode laser bar can also vary. In addition, the width of the emitters and light divergence can vary. The emitter spacing and the number of emitters can be customized during the manufacturing process.

FIG. 24B shows a perspective view of one embodiment of a diode laser bar 330 that can be used for the diode laser bar 315 in FIG. 24A. The diode laser bar 330 has length L of around 1 cm, a width W of around 1 mm, and a thickness T of around 0.0015 mm. The depiction of FIG. 24B shows 12 emitters 332, each of which emits radiation 334 as shown in FIG. 24B. The diode laser bar 330 can be placed within the device 310 of FIG. 24A so that the side S of the diode laser bar 315 is oriented as shown in FIG. 24A. The emitters, therefore, emit radiation downward toward the skin 319 in the embodiment of FIG. 24A.

Referring again to FIG. 24A, the plate 317 can be of any type, such as those set forth above, in which light from an EMR source can pass through the plate 317. In one embodiment, the plate 317 can be a thin sapphire plate. In other embodiments, other optical materials with good optical transparency and high thermal conductivity/diffusivity, such as, for example, diamond, can be used for the plate 317. The plate 317 can be used to separate the diode laser bar 315 from the patient's skin 319 during use. In addition, the plate 317 can provide cooling or heating to the patient's skin, if desired. The area in which the plate 317 touches the patient's skin can be referred to as the treatment window. The diode laser bar 315 can be disposed within the housing 313 such that the emitters are in close proximity to the plate 317, and therefore in close proximity to the patient's skin when in use.

In operation, one way to create islets of treatment is to place the housing 313, including the diode laser bar 315, in close proximity to the skin, and then fire the laser. Wavelengths near 1750-2000 nm and in the 1400-1600 nm range can be used for creating subsurface islets of treatment with minimal effect on the epidermis due to high water absorption. Wavelengths in the 290-10,000 can be used in some embodiments, while in other wavelengths in the 900-10,000 nm range can be used for creating surface and subsurface islets on the skin. Without moving the hand piece across the skin, a series of treatment islets along a line can be formed in the skin. FIG. 22 shows one possible arrangement 290 of islets on the surface of the skin 280 from the use of such a diode laser bar, where the diode laser bar 315 is pulsed as it moves over the skin in direction A of FIG. 24A.

In another embodiment, the user can simply place the hand piece in contact with the target skin area and move the hand piece over the skin while the diode laser is continuously fired to create a series of lines of treatment. For example, using the diode laser bar 330 of FIG. 24B, 12 lines of treatment would appear on the skin (one line for each emitter).

In another embodiment, an optical fiber can couple to the output of each emitter of the diode laser bar. In such an embodiment, the diode laser bar need not be as close to the skin during use. The optical fibers can, instead, couple the light from the emitters to the plate that will be in close proximity to the skin when in use.

FIG. 24C shows another embodiment, which uses multiple diode laser bars to create a matrix of islets of treatment. As shown in FIG. 24C, multiple diode laser bars can be arranged to form a stack of bars 325. In FIG. 24C, for example, the stack of bars 325 includes five diode laser bars. In a similar manner as set forth above in connection with FIG. 24A, the stack of bars 325 can be mounted in the housing 313 of a hand piece H101 with the emitters very close to a cooling plate 317.

In operation, the hand piece 310 of FIG. 24C can be brought close to the skin surface 319, such that the cooling plate 317 is in contact with the skin. The user can simply move the hand piece over the skin as the diode lasers are pulsed to create a matrix of islets of treatment in the skin. The emission wavelengths of the stacked bars need not be identical. In some embodiments, it may be advantageous to mix different wavelength bars in the same stack to achieve the desired treatment results. By selecting bars that emit at different wavelengths, the depth of penetration can be varied, and therefore the islets of treatment spot depth can also be varied. Thus, the lines or spots of islets of treatment created by the individual bars can be located at different depths.

During operation, the user of the hand piece 310 of FIG. 24A or 24C can place the treatment window of the hand piece in contact with a first location on the skin, fire the diode lasers in the first location, and then place the hand piece in contact with a second location on the skin and repeat firing.

In addition to the embodiments set forth above in which the diode laser bar(s) is located close to the skin surface to create islets of treatment, a variety of optical systems can be used to couple light from the diode laser bar to the skin. For example, with reference to FIGS. 24A and 24C, imaging optics can be used to re-image the emitters onto the skin surface, which allows space to be incorporated between the diode laser bar 315 (or the stack of bars 325) and the cooling plate 317. In another embodiment, a diffractive optic can be located between the diode laser bar 315 and the output window (i.e., the cooling plate 317) to create an arbitrary matrix of treatment spots. Numerous exemplary types of imaging optics and/or diffractive optics that can also be used in this embodiment are set forth in the section entitled Devices and Systems for Creation of Islets (Example 2) above.

Another embodiment is depicted in FIG. 24D. In this embodiment, the housing 313 of the hand piece 310 includes a stack 325 of diode laser bars and a plate 317 as in previous embodiments. This embodiment, however, also includes four diffractive optical elements 330 disposed between the stack 250 and the plate 317. In other embodiments, more or fewer than four diffractive optical elements 330 can be included. The diffractive optical elements 330 can diffract and/or focus the energy from the stack 325 to form a pattern of islets of treatment in the skin 319. In one embodiment, one or more motors 334 is included in the hand piece 310 in order to move the diffractive optical elements 330. The motor 334 can be any suitable motor, including, for example, a linear motor or a piezoelectric motor. In one embodiment, the motor 334 can move one or more of the diffractive optical elements 330 in a horizontal direction so that those elements 330 are no longer in the optical path, leaving only one (or perhaps more) of the diffractive optical elements 334 in the optical path. In another embodiment, the motor 334 can move one or more of the diffractive optical elements 330 in a vertical direction in order to change the focusing of the beams.

In operation, by incorporating more than one diffractive optics 330 in the hand piece 310 along with a motor 334 for moving the different diffractive optics 330 between the stack 325 of diode laser bars and the plate 317, the diffractive optics 330 can be moved in position between the stack 325 and the cooling plate 317 in order to focus the energy into different patterns. Thus, in such an embodiment, the user is able to choose from a number of different islets of treatment patterns in the skin through the use of the same hand piece 310. In order to use this embodiment, the user can manually place the hand piece 310 on the target area of the skin prior to firing, similar to the embodiments described earlier. In other embodiments, the hand piece aperture need not tough the skin. In such an embodiment, the hand piece may include a stand off mechanism (not shown) for establishing a predetermined distance between the hand piece aperture and the skin surface.

FIG. 24E shows another embodiment. In this embodiment, optical fibers 340 are used to couple light to the output/aperture of the hand piece 310. Therefore, the diode laser bar (or diode laser bar stacks or other light source) can be located in a base unit or in the hand piece 310 itself. In either case, the optical fibers couple the light to the output/aperture of the hand piece 310.

In the embodiment of FIG. 24E, the optical fibers 340 may be bonded to the treatment window or cooling plate 317 in a matrix arrangement with arbitrary or regular spacing between each of the optical fibers 340. FIG. 24E depicts five such optical fibers 340, although fewer or, more likely, more optical fibers 340 can be used in other embodiments. For example, a matrix arrangement of 30 by 10 optical fibers could be used in one exemplary embodiment. In the depicted embodiment, the diode laser bar (or diode laser bar stacks) is located in the base unit (which is not shown). The diode laser bar (or diode laser bar stacks) can also be kept in the hand piece. The use of optical fibers 340 allow the bar(s) to be located at an arbitrary position within the hand piece 310 or, alternatively, outside the hand piece 310.

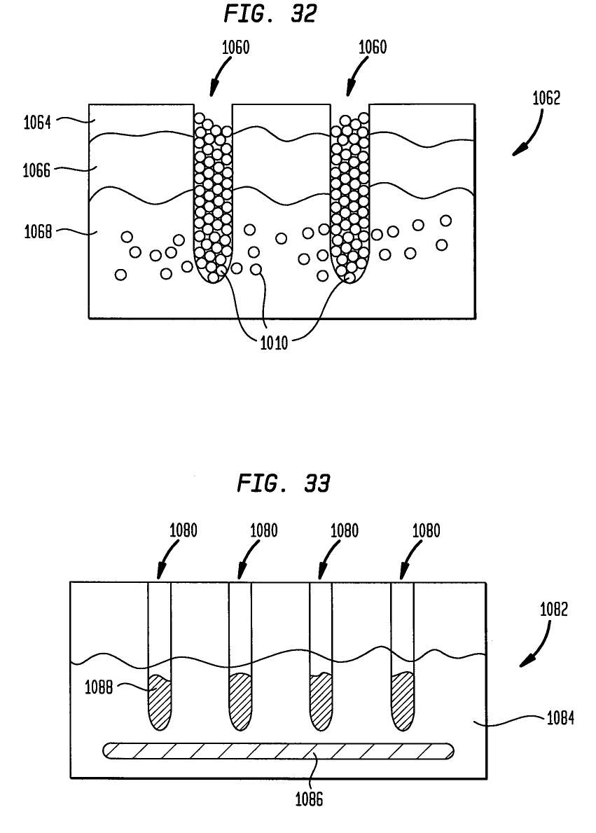

As an example of an application of a diode laser bar to create thermal damage zones in the epidermis of human skin, a diode laser bar assembly, as depicted in FIGS. 26 and 32, emitting at a wavelength λ=1.47 μm, was constructed and applied to human skin ex vivo at room temperature in a stamping mode (that is, in a mode where the assembly does not move across the skin during use). The diode bar assembly had a sapphire window, which was placed in contact with the skin and the laser was pulsed for about 10 ms. The treated skin was then sliced through the center of the laser-treated zones to reveal a cross-section of the stratum corneum, epidermis and dermis. The resulting thermal damage channels were approximately 100 μm in diameter and 125-150 μm in depth for the 10 mJ per channel treatments.

N. Solid State Laser Embodiments

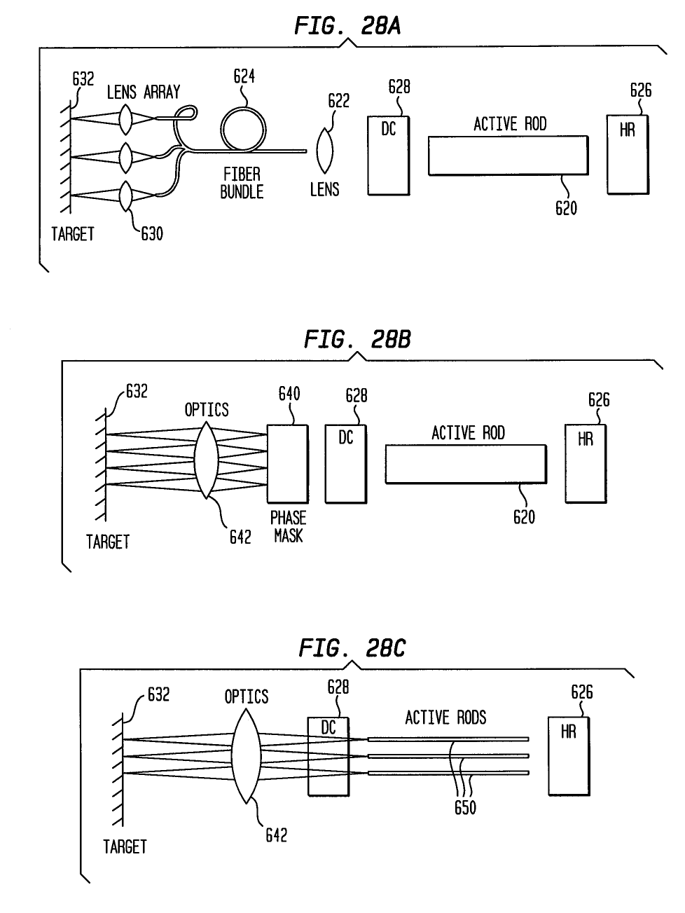

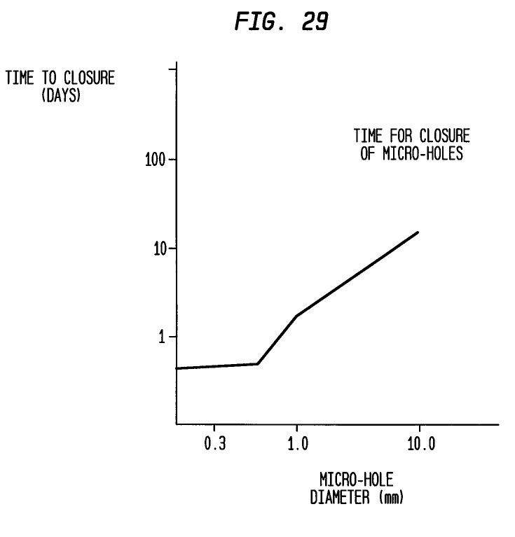

FIGS. 28A-C show additional embodiments. FIG. 28A shows an embodiment in which the apparatus includes a laser source 620, focusing optics (e.g., a lens) 622, and a fiber bundle 624. The laser source 620 can be any suitable source for this application, for example, a solid state laser, a fiber laser, a diode laser, or a dye laser. In one embodiment, the laser source 620 can be an active rod made from garnet doped with rare earth ions. The laser source 620 can be housed in a hand piece or in a separate base unit.

In the exemplary embodiment as in FIG. 28A, the laser source 620 is surrounded by a reflector 626 (which can be a high reflector HR) and an output coupler 628 (OC). In other embodiments, the reflector 626 and the coupler 628 are not used. Various types and geometries of reflectors can be used for reflector 626. The fiber bundle 624 is located optically downstream from the lens 622, so that the optical lens 622 directs and focuses light into the fiber bundle 624.

In one embodiment, an optical element 630, such as a lens array, can be used to direct and output the EMR from the fiber bundle 624 in order to focus the EMR onto the patient's skin 632. The optical element 630 can be any suitable element or an array of elements (such as lenses or micro lenses) for focusing EMR. In the embodiment of FIG. 28A, the optical element 630 is a micro lens array. In other embodiments, an optical element 630 might not be used. In such an embodiment, the outputs of the fibers in the fiber bundle 624 can be connected to one side of a treatment window (such as a cooling plate of the apparatus), where the other side of the treatment window is in contact with the patient's skin 632.

In operation, the laser source 620 generates EMR and the reflector 626 reflects some of it back toward the output coupler 628. The EMR then passes through the output coupler 628 to the optical lens 622, which directs and focuses the EMR into the fiber bundle 624. The micro lens array 630 at the end of the fiber bundle 624 focuses the EMR onto the patient's skin 632.

FIG. 28B shows another embodiment. In this embodiment, the apparatus includes a laser source 620 and a phase mask 640. The laser source 620 can be any type of laser source and can be housed in a hand piece or in a separate base unit, such as in the embodiment of FIG. 28A. In one embodiment, the laser source 620 can be an active rod made from garnet doped with rare earth ions. Also like the embodiment of FIG. 28A, the laser source 620 can be surrounded by a reflector 626 (which can be a high reflector HR) and can output EMR into an output coupler 628 (OC).

The embodiment of FIG. 28B includes a phase mask 640 that is located between the output coupler 628 and an optical element 642. The phase mask 640 can include a set of apertures that spatially modulate the EMR. Various types of phase masks can be used in order to spatially modulate the EMR in order to form islets of treatment on the patient's skin 632. The optical element 642 can be any suitable element or an array of elements (such as lenses or micro lenses) that focuses the EMR radiation onto the patient's skin 632. In embodiment of FIG. 28B, the optical element 642 is a lens.

In operation, the laser source 620 generates EMR and the reflector 626 reflects some of it back toward the output coupler 628. The EMR then passes through the output coupler 628 to the phase mask 640, which spatially modulates the radiation. The optical element 642, which is optically downstream from the phase mask 640 so that it receives output EMR from the phase mask 640, generates an image of the apertures on the patient's skin.

FIG. 28C shows another embodiment. In this embodiment, the apparatus includes multiple laser sources 650 and optics to focus the EMR onto the patient's skin 632. The multiple laser sources 650 can be any suitable sources for this application, for example, diode lasers or fiber lasers. For example, the laser sources 650 can be a bundle of active rods made from garnet doped with rare earth ions. The laser sources 650 can optionally be surrounded by a reflector and/or an output coupler, similar to the embodiments of FIGS. 28A and 28B.

In the embodiment of FIG. 28C, an optical element 642 can be used for focusing the EMR onto the patient's skin 632. Any suitable element or an array of elements (such as lenses or micro lenses) can be used for the optical element 642. The optical element, for example, can be a lens 642.

In operation, the bundle of lasers 650 generate EMR. The EMR is spatially modulated by spacing apart the laser sources 650 as shown in FIG. 28C. The EMR that is output from the laser sources 650, therefore, is spatially modulated. This EMR passes through the output coupler 628 to the optical element 642, which focuses the EMR onto the patient's skin 632 to form islets of treatment.

In the exemplary embodiment of FIGS. 28A-C, which each use a garnet laser rod doped with rare earth ions, the spectral range of electromagnetic radiation is about 400-3000 nm, the energy exposure is up to about 1000 J/cm², the laser pulse duration is from about 10 ps to 10 s, and the fill factor is from about 1% to 90%.

O. Consumer-Oriented Products and Methods

Other embodiments can be used in consumer devices as well as professional devices, depending on the application.

### IV. Applications For The Use of Micro-Holes And Other Micro-Structures In Tissue

A. Applications Generally

When a micro-hole is created in tissue in vivo, healing processes will cause the micro-holes to heal and, if open through the surface, close. If the micro-hole extends from the surface of skin tissue and into the tissue, the time required to close the micro-hole is roughly proportional to the diameter of the opening of the micro-hole at the surface. A smaller opening will heal more quickly, and a larger hole will take longer to heal. FIG. 29 illustrates a general approximation of the time it takes for micro-holes of varying sizes to close at the surface of skin tissue.

The closure of the micro-holes provides benefits such as protection from infection. Thus, a quickly closed hole can help reduce the chances of infection as compared to a larger hole that is open longer. A treatment that employs relatively smaller holes, therefore, can provide safety benefits over similar procedures using larger holes. If the hole is small enough, a fairly aggressive ablative skin-rejuvenation or other procedure (for example, a procedure having a high density of micro-holes or deep micro-holes or both) can be performed on a person with minimal risk of infection, because, as demonstrated in FIG. 29, micro-holes having diameters on the order of approximately 3.0-30 μm will be closed in approximately a half day or less.

Generally, by using smaller micro-holes during treatments, the overall healing time is reduced. This has many potential treatment benefits, such as allowing the person treated to return for additional rounds of treatment sooner, and completing a course of treatment more quickly. Unlike currently available treatments, the faster closure of the micro-holes also allows the person treated to resume regular activities such as applying cosmetics or swimming, in some cases within less than a day.

In some embodiments, micro-structures that result in a sterile or semi-sterile environment are possible. For example, micro-holes that are too small to pass certain foreign substances are possible. Additionally, in some embodiments, the ablative process may result in heat transfer to tissue surrounding the micro-structure or forming the wall of the micro-structure, and that tissue may shrink as a result of the heating, further decreasing the size of the micro-structure and contributing to the fast healing time.

Similarly, in other embodiments, the micro-structures may result in a bloodless wound or may restrict blood loss. For example, micro-holes may be created that are too small for blood to escape or are so small that blood loss is minimal.

Additionally, micro-holes can be used in many other applications, including without limitation:
1. to treat the apocrine gland, sebaceous glands or other glands;
2. to provide skin rejuvenation;
3. to provide skin resurfacing;
4. to irradiate tissue with optical radiation, other EMR, or other forms of energy, following the formation of micro-holes in the tissue;
5. to treat various conditions, such as acne;
6. to treat or reduce fat;
7. to provide permanent or temporary hair removal;
8. to deliver a chromophore that is subsequently heated with EMR or other energy, for example, to remove hair, treat sebaceous glands;
9. to increase the permeability of tissue such as the stratum cornea;
10. to provide tattoos;
11. to provide permanent or semi-permanent cosmetics;
12. to provide permanent or semi-permanent protection from ultraviolet light;
13. to deliver drugs, medications, vitamins, and/or other substances;
14. to deliver fillers, such as, for example, collagen, silicon or fat used in cosmetic surgeries;
15. to deliver chemically active substances;
16. to deliver chemically or biologically inert substances; and
17. to deliver a clearing agent, such as glycerol, that causes the tissue to have increased translucence and/or transparency;
18. to treat tissue with extra-cellular matrix (ECM)
19. to treat tissue with stem cells
20. to treat tissue with proteins, for example, Wnt proteins, or to stimulate pathways mediated by proteins;
21. to treat tissue with β-catenin and/or stimulate β-catenin activity;
22. to generate new tissue; and
23. to generate new structures within tissue, such as hair follicles.

Many other applications and uses are possible. The following sections provide additional detailed description of several exemplary applications.

B. Skin Rejuvenation and Tightening Using Micro-Grooves.

By forming an array of micro-grooves in the skin, skin can be tightened, rejuvenated, and wrinkles (both deep and superficial), fine lines and rhytides can be eliminated from the skin. By removing a percentage of the tissue in a treatment area (e.g., 30%-40% of the tissue measured by volume or surface area), significantly less tissue remains in the treatment area after ablation than prior to ablation. Thus, tissue can be tightened or reshaped by at least two methods. First, the natural healing processes associated with the tissue, such as skin tissue. Second, additional mechanical manipulation of the tissue. Furthermore, the process can be used to improve the micro-texture of the tissue.

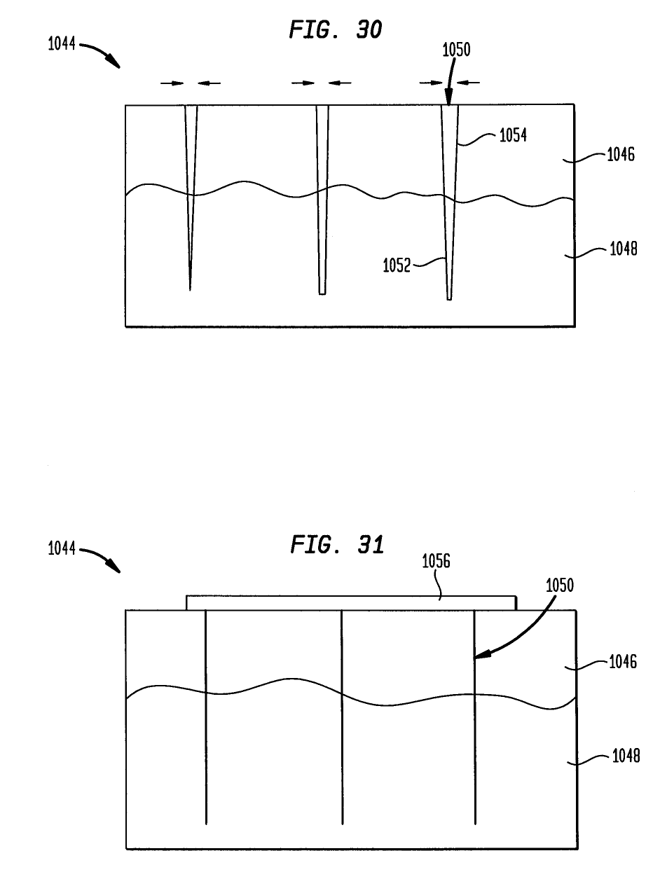

For example, referring to FIGS. 30 and 31, tissue can be removed from a groove 1050 and the walls 1052 and 1054 of the grooves subsequently pushed together to form a tissue surface having a reduced area. In FIG. 30, the walls 1052 and 1054 of groove 1050 have been partially pressed together to remove the void that originally formed groove 1050. Groove 1050 was originally formed in a tissue volume 1044 and extended though an epidermal layer 1046 of the tissue and into a dermal layer 1048. Referring to FIG. 31, when the walls of the grooves 1050 are pushed together, they may be held in place by many different methods. For example, an adhesive surgical film 1056 or surgical tape can be applied after manually compressing the walls of the grooves together. Alternatively, a film can be applied that adheres to the tissue and subsequently shrinks to compress the walls of the grooves together. Additionally, a spray can be applied that shrinks and fixes the tissue in place.

The treated areas of tissue can then be reshaped. For example, for skin tissue, the treated areas can be manipulated to tighten the skin or lift the skin or otherwise reshape the skin. Ablated grooves can also be used to reduce the area of skin tissue following various invasive procedures, such as liposuction.

Using arrays of micro-grooves to tighten tissue has several advantages in some applications over both ablative and non-ablative fractional techniques. For example, for wrinkle removal, forming EMR-treatment islets in the form of an array of circular islands of damage does not alter the structural integrity of the tissue. Thus, such methods rely on the healing response alone to remove the wrinkle. By ablating grooves of tissue from the skin, however, the healing response is still achieved, and the integrity of the tissue in which the wrinkle resides is altered such that it can be mechanically altered to better remove the wrinkle. Further, in the case of micro-grooves or similar micro-structures, there is no bulk damage to any portion of the tissue but a portion of the epidermis is damaged, which may improve the results when compared to non-ablative, non-fractional techniques for wrinkle removal.

Additional methods may combine the use of micro-grooves or other micro-structures with the use of an injected muscle management substance such as Botulinum Toxin Type A (e.g., Botox®), or other similar substances. Using micro-grooves in combination with the application of such substances increases the length of the effect of the treatment when compared to the application of Botox® alone. Additionally, such muscle management substances can decrease the stress and/or tension on the treated skin tissue during the healing process to produce a better result.

In addition to mechanically manipulating tissue, a subject being treated can be positioned to allow gravity to stretch the skin prior to treatment, such as lying face up or with the top of the head tilted at a downward angle when treating the face and/or neck.

C. Ablation Islets For Skin Rejuvenation and Wrinkle Removal

Skin rejuvenation as well as the removal of wrinkles, fine lines and rhytides can be accomplished by other embodiments in addition to the embodiments involving grooves above. For example, the healing process resulting from an array of ablated micro-holes will produce rejuvenated skin, such as skin with fewer age spots or other pigmented lesions and skin with smoother texture. The healing process will also reduce the number and degree of wrinkles, fine lines and rhytides.

Fractional ablative methods may have one or more advantages over existing non-ablative skin rejuvenation and wrinkle removal techniques, including, without limitation, less pain, shorter down time, higher safety margins, deeper treatments, and improved results. Exemplary treatments of the eyelids, upper lip, acne scarring and peri-orbital wrinkles are performed using an Er:YSGG laser at 2790 nm with a pulse width of 2 or 5 ms and a fluence of 6-9 mJ per beam. Alternatively, an Er:YAG laser with a wavelengths of 2940 nm, a pulsewidth of 300 μm, and a fluence of 3-6 mJ per beam can be used. (See Table B for additional associated parameters.) Using the parameters of Table B above, multiple passes may be preferable, e.g., 6 passes with passes 1-2 at a fluence of 5 mJ/beam, and passes 3-6 at a reduced fluence 3 mJ/beam. Many other combinations of parameters are possible for skin rejuvenation, wrinkle removal, and other applications.

Additionally, skin rejuvenation and wrinkle removal can be achieved by the targeted stimulation of hyaluronic acid in skin tissue. The creation of lattices of micro-holes can result in the promotion of production of hyaluronic acid as a result of the healing response of tissues to thermal stress or thermal shock (short- to medium-term effect). Repeating treatments in regular intervals can maintain the level of hyaluronic acid and as a result maintain improved skin appearance.

In some embodiments, skin rejuvenation may result from the introduction of certain types of fillers that enhance the mechanical and optical properties of the tissue. These embodiments are discussed in greater detail below.

D. Delivery, Absorption and Extraction of Substances Through Micro-Structures

Substances can be extracted or delivered using various methods, including absorption, vibration, other mechanical stimulation (such as massaging of the tissue or applying positive or negative pressure), applying electrical or magnetic fields, application of a jet spray and application of acoustic energy such as ultrasound. For example, a magnetic field can be applied to magnetized particles that are then forced into the micro-holes or that or pulled from the micro-holes. Additionally, a chromophore in the micro-holes or delivered via the micro-holes can be heated using the magnetic field or other energy rather than using EMR. Substances can be delivered as solids, liquids, and particulates and crystals applied as part of a jet spray system. The substances can be elements, compounds, mixtures, compositions, suspensions, and may include components in different phases, such as small solid particulates in a liquid. When introduced into a cavity of a micro-structure, the substance can remain in the micro-structure or disburse into the tissue, e.g., by dissolving, transportation across membranes in the tissue, or other means.

1. Tissue Permeability

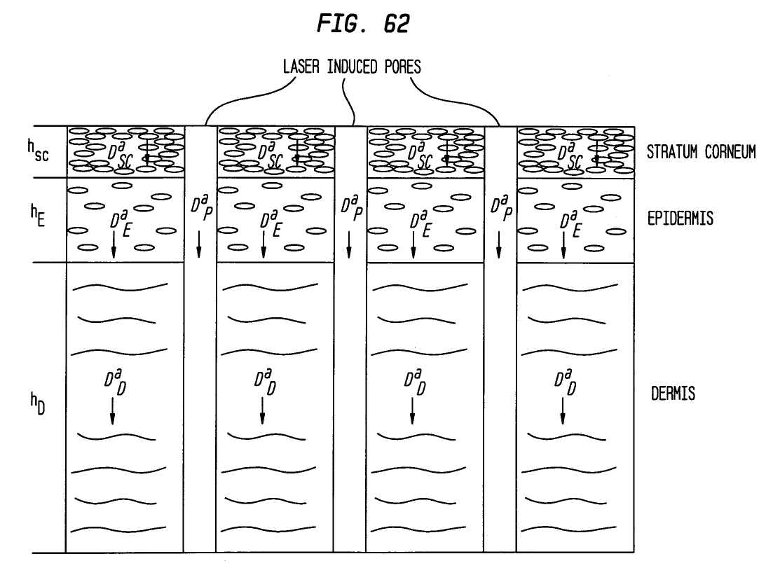

Referring to FIG. 62, a model of skin tissue with regularly-spaces micro-holes extending from the skin surface to the dermis, demonstrates that the micro-holes act as pores that facilitate absorption. The model demonstrates that the total permeability coefficient, PT, for an agent diffusing through the skin is defined as

$$\frac{1}{{P}_{T}} = \frac{1}{{P}_{SC}} + \frac{1}{{P}_{E}} + \frac{1}{{P}_{D}} , \qquad ( 3 )$$

where P_{SC}, P_{E}, and P_{D} are the permeability coefficients for the agent diffusing through the sin respectively for SC, epidermis, dermis.

Because all three skin layers are perforated by an erbium laser, all three permeation coefficients may be presented in the form assuming that each permeability coefficient is the summation of a normal pathway (0) and a pore pathway (p) weighted by the pores filling factor fᵢ

P_{SC}=(1−f_{SC})P_{SC}⁰+f_{SC}P_{SC}^{p}, (6)

P_{E}=(1−f_{E})P_{E}⁰+f_{E}P_{E}^{p}, (7)

P_{D}=(1−f_{D})P_{D}⁰+f_{D}P_{D}^{p}, (8)

where

$$\begin{matrix}{P}_{j}^{i} = \frac{{D}_{j}^{i}}{{h}_{j}} , & ( 9 ) \\ {f}_{j} = \frac{π {d}_{j}^{2}}{4 S} {N}_{j} & ( 10 )\end{matrix}$$

i=0, p and j=SC, E, D: Dⱼⁱ for i=0 is the diffusion coefficient of an agent in the corresponding intact part of tissue layer, and for i=p is the diffusion coefficient of this agent in the corresponding damaged part of tissue layer; hⱼ is the thickness of the skin layer; dⱼ is the diameter of a circular micro-structure (in this case a micro-hole); Nⱼ is the number of such micro-structures with a skin surface area S. Thus, to estimate skin permeation when perforated with an erbium laser, it follows that:

$${P}_{T} = \frac{{P}_{SC} {P}_{E} {P}_{D}}{{P}_{E} {P}_{D} + {P}_{SC} {P}_{D} + {P}_{SC} {P}_{E}} \qquad ( 11 )$$

For simplicity, all micro-holes are presumed to be of the equal diameter and running without change of their diameter through all three skin layers, i.e., fᵢ=f; and the diffusion coefficient of the agent (α) along a micro-hole crossing all skin layers is equal to its diffusion in water Dⱼ^{p}=D_{w}^{α}. Thus, f for laser damaging should be in the range of 0.01-0.2. The permeability of intact stratum corneum is a few orders less than water for any agent. For example for small molecules, such as glycerol, propylene glycol, diffusion coefficient in stratum corneum is close to water diffusivity in the stratum corneum, i.e., D_{α}=3×10⁻¹⁰ cm²/s. For living epidermis the typical diffusivity of a number of agents is of D_{α}=3×10⁻⁸ cm²/s. Two orders higher diffusivity of the living epidermis in comparison with the stratum corneum is due to a more permeation ability of epidermal cell membrane, which is similar to permeability of membranes of other epithelial cells. For dermis the diffusivity is approximately equal to diffusivity of any fibrous tissue, D_{α}=3×10⁻⁶ cm²/s, that is close to diffusivity of small molecules in water.

Accounting for above estimations, permeability is approximated by the following equations:

$$\begin{matrix}{P}_{SC} \approx f \frac{{D}_{w}^{a}}{{h}_{SC}} , & ( 12 ) \\ {P}_{E} \approx 2 f \frac{{D}_{w}^{a}}{{h}_{E}} , & ( 13 ) \\ {P}_{D} \approx \frac{{D}_{w}^{a}}{{h}_{D}} , & ( 14 )\end{matrix}$$

Substituting these approximate equations into the equation (11) above gives the following:

$${P}_{T} \approx \frac{2 {fD}_{w}^{a}}{{h}_{SC} + {h}_{E} + 2 {fh}_{D}} . \qquad ( 15 )$$

The typical values of the human skin layers thicknesses are the following h_{SC}≈10-20 μm, h_{E}≈100-200 μm, and h_{D}≈1000-2000 μm. Thus, for thick skin perforated with a high filling factor, not less than 0.1, the total skin permeability is defined by dermis only. For the small filling factors, of 0.01 and less, and rather thin dermis layer, the total skin permeation is proportional to the filling factor and depends inversely on thicknesses of stratum corneum and epidermis. This formula qualitatively describes the experimental fact that permeation of laser ablated skin can be saturated when the percentage of the ablated area is approximately 13%.

As an example, using a low molecular weight compound, the total permeability significantly increases for skin containing micro-holes in comparison with intact skin: 54 fold for thin skin; 43 fold for medium thickness skin, and 31 fold for thick skin models when filling factor changes from 0 to 0.01. Because dermal thickness dominates for all skin models and agent's diffusivity in intact dermis is only one order less than in water the total permeability increases approximately equally, 10.7-11.5-fold, with a fill factor increase from 0.01 to 1.

Based on the above analysis, all of the methods of physical deliver described herein, such as iontophoresis, sonophoresis, electroosmosis, laser-induced pressure waves, and topical application of alcohol, and other chemical permeation enhancers can be used in combination with the formation of micro-structures in the skin. Similarly, the existence of a micro-hole or other micro-structure in the skin will allow various physical techniques such as mechanical compression, stretching, and/or fast flow sprays may, to be used to deliver particles, suspensions of particles, and other substances and compositions into the skin.

Following delivery of a substance, including fillers, chromophores, drugs and other substances, an occlusive bandage, or other barrier, can be fixed to the tissue to retain the substance within the micro-voids and/or to reduce or prevent vapor exchange through the tissue.

### 2. Diffusion and Percolation

Transport of chemical agents into skin for improvement of its appearance (smooth reflectivity) can be described by the mathematical model based on passive agent diffusion. Assuming: 1) concentration diffusion, i.e. exchange flux of the agent into skin and water from tissue is proportional to the gradient of concentration of osmotic active agent in the point; and 2) diffusion coefficient is constant over the entire sample volume.

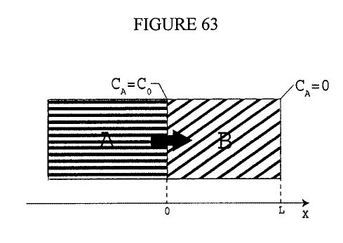

The diffusion process is considered in a bounded semi-infinite space. At start time t=0 concentration of the agent A in the object is equal to 0. Concentration of the agent A on the boundary of the object at the same time is C₀ and do not change with time. Sample layer B has a limited thickness L, for x>L concentration of the agent A is equal to 0 at any time (FIG. 63).

We will find a distribution function of the concentration of the agent A depending on coordinate and time scale. Sample is considered as a flat-parallel of thickness L. For the simplicity, we will solve a one-dimensional problem, basing on the solution of the Fick's second law:

$$\frac{\partial C ( x ; t )}{\partial t} = D \frac{{\partial }^{2} C ( x ; t )}{\partial {x}^{2}} \qquad ( 16 )$$

where C(x; t) is the concentration of the index-matching or mismatching agent in the skin, g/ml; D is the diffusion coefficient, cm²/s; t is the time of diffusion process, s; and x is the spatial coordinate.

The boundary and initial conditions for Eq. (16) are the following:

$$\{ \begin{matrix}\frac{\partial C ( x ; t )}{\partial t} = D \frac{{\partial }^{2} C ( x ; t )}{\partial {x}^{2}} \\ C ( 0 ; t ) = {C}_{0} \\ C ( L ; t ) = 0 \\ C ( x ; 0 ) = 0 , x > 0\end{matrix} \qquad ( 17 )$$

Solution of this problem we will find in the form of sum of two functions:

C(x;t)=v(x;t)+α(x) (18)

Here v(x; t) describes time-dependent agent distribution, and α(x) is the time-independent agent distribution, α(x)=κx+b, where coefficients k and b can be found from the boundary conditions:

$$\begin{matrix}α ( 0 ) = k 0 + b = {C}_{0} ⇒ b = {C}_{0} ; & ( 19 ) \\ α ( L ) = kL + {C}_{0} = 0 ⇒ k = - \frac{{C}_{0}}{L} . & \end{matrix}$$

### Thus:

$$α ( x ) = - \frac{{C}_{0}}{L} x + {C}_{0} \qquad ( 20 )$$

We will evaluate function v(x; t):

v(x;t)=C(x;t)−α(x),

v(x,0)=C(x;0)+C₀x/L−C₀=0+C₀x/L−C₀,

v(0;t)=0,v(L;t)=0 (21)

The expression for function v(x; t) can be written as:

$$\{ \begin{matrix}\frac{\partial v ( x ; t )}{\partial t} = D \frac{{\partial }^{2} v ( x ; t )}{\partial {x}^{2}} ; \\ v ( x ; 0 ) = \frac{{C}_{0}}{L} x - {C}_{0} ; \\ v ( 0 ; t ) = 0 ; \\ v ( L ; t ) = 0.\end{matrix} \qquad ( 22 )$$

Solution of Eq. 16 can be written in the form:

v(x,t)=X(x)·T(t) (23)

Using Eqs. (22) and (23), it is possible to find:

$$\frac{1}{D} \frac{{T}^{′}}{T} = \frac{{X}^{″}}{X} = - λ ,$$

where λ=const, since left part of equation depends on t only, and right part depends on x. It follows that:

X″+λX=0

T′+DλT=0

The boundary conditions give:

X(0)=0,X(L)=0

Hence, in order to defining X(x) we have characteristic constants problem:

X″+λX=0,X(0)=0,X(L)=0 (24)

This problem has nontrivial solutions

$${X}_{n} ( x ) = sin \frac{n π x}{L}$$

only for

$${λ}_{n} = {( \frac{n π}{L} )}^{2} ,$$

(n=1; 2; 3 . . . ). The values of λₙ are corresponding to Tₙ(t)=Cₙe^{-Dλ}^{ₙ}^{t}, where Cₙ—are indefinite coefficients. Functions

$${v}_{n} ( x ; t ) = {X}_{n} ( x ) {T}_{n} ( t ) = {C}_{n} {e}^{- D {λ}_{n} t} sin \frac{n π x}{L}$$

are particular solutions of differential equation with zero boundary conditions. The presented above expression can be replaced by the series:

$$v ( x ; t ) = {\sum }_{n = 1}^{∞} {C}_{n} {e}^{- ( \frac{n π}{L} ) Dt} \qquad ( 25 )$$

All terms of series satisfy the boundary conditions, hence function v(x; t) also satisfies these conditions. The realization of initial conditions gives:

$$v ( x ; 0 ) = \frac{{C}_{0}}{L} x - {C}_{0} = {\sum }_{n = 1}^{∞} {C}_{n} sin \frac{n π x}{L} , \qquad ( 26 )$$

where Cₙ are Fourier coefficients of function v(x;0) being expanded in sins on the interval (0; 1):

$${C}_{n} = \frac{2}{L} {\int }_{0}^{L} ( \frac{{C}_{0}}{L} x - {C}_{0} ) sin \frac{n π x}{L} d x \qquad ( 27 )$$

Calculations give:

$${C}_{n} = - \frac{2 {C}_{0}}{π n} .$$

The initial problem assumes the following distribution function of concentration:

$$C ( x ; t ) = v ( x ; t ) + α ( x ) = - \frac{2 {C}_{0}}{π} {\sum }_{n = 1}^{∞} \frac{1}{n} {e}^{- {( \frac{π n}{L} )}^{2} Dt} sin \frac{n π x}{L} - \frac{{C}_{0}}{L} x + {C}_{0} \qquad ( 28 )$$

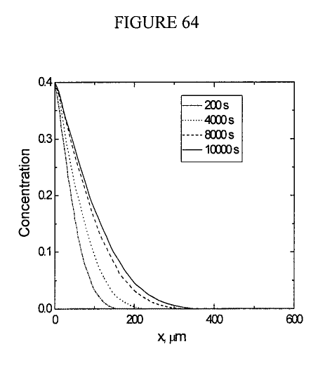

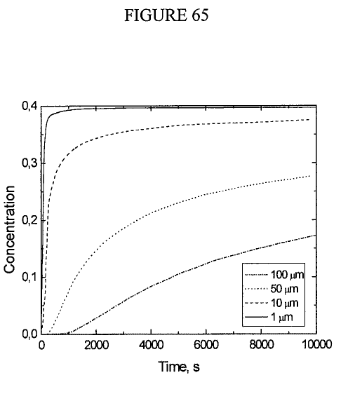

FIG. 64 illustrates distribution of glycerol concentration along the sample depth. The temporal distributions of glycerol concentration are presented in FIG. 65 All dependences were obtained using Eq. (28) for the sample of thickness 1 mm, diffusion coefficient D=0.8×10⁻⁸ cm²/s, C₀=0.4.
Integration of Eq. (28) over x with limits of integration from 0 to L accounting for C(x;t)=v(x;t)+α(x), gives:

$${\int }_{0}^{L} C ( x ; t ) d x = {\int }_{0}^{L} v ( x ; t ) d x + {\int }_{0}^{L} α ( x ) d x .$$

### Thus:

$$\begin{matrix}{\int }_{0}^{L} v ( x ; t ) d x = \frac{2 {C}_{0} L}{{π}^{2}} {\sum }_{n = 1}^{∞} \frac{1}{{n}^{2}} {e}^{- {( \frac{π \cdot n}{L} )}^{2} D \cdot t} ( cos n π - 1 ) & ( 29 ) \\ {\int }_{0}^{L} α ( x ) d x = \frac{1}{2} {C}_{0} L & ( 30 ) \\ {\int }_{0}^{L} C ( x ; t ) d x = \frac{2 {C}_{0} L}{{π}^{2}} {\sum }_{n = 1}^{∞} \frac{1}{{n}^{2}} {e}^{- {( \frac{π \cdot n}{L} )}^{2} D \cdot t} ( cos n π - 1 ) + \frac{1}{2} {C}_{0} L & ( 31 )\end{matrix}$$

The penetration of the agent in the skin leads to increasing of the refractive index of the interstitial space that may improve skin appearance (smoothness and intensity of from-depth reflectance). Estimation of refractive index value of the interstitial space in dependence on diffusion time can be made on the basis of the law of Gladstone and Dale. According to this law, the refractive index of non-interacting liquids is the sum of the products of refractive indices of the separated components weighted by their volume fractions:

$${n}_{Σ} = {\sum }_{i}^{ } {n}_{i} {C}_{i}$$

where n_{Σ} is the refractive index of the multi-component composition of non-interacting liquids, nᵢ and Cᵢ are the refractive index and the volume fraction of each component. In the case of bi-phase solutions (in our case—interstitial fluid and glycerol), the law of Gladstone and Dale can be written as:

$${n}_{i} ( t ) = ( 1 - C ( t ) ) {n}_{inter} + C ( t ) {n}_{a} , {\sum }_{i}^{ } {C}_{i} = 1 , \qquad ( 32 )$$

where n_{int er} is the refractive index of interstitial fluid in the initial time and n_{α} is the refractive index of the diffusing agent (glycerol).

### From Eq. (32):

$$C ( t ) = \frac{{n}_{i} ( t ) - {n}_{inter}}{{n}_{a} - {n}_{inter}} , \qquad ( 33 )$$

On the other hand:

$$C ( t ) = \frac{2 {C}_{0}}{{π}^{2}} {\sum }_{n = 1}^{∞} \frac{1}{{n}^{2}} {e}^{- {( \frac{π \cdot n}{L} )}^{2} D \cdot t} ( cos n π - 1 ) + \frac{1}{2} {C}_{0} , \qquad ( 34 )$$

hence:

$$\frac{{n}_{i} ( t ) - {n}_{int er}}{{n}_{a} - {n}_{int er}} = \frac{2 {C}_{0}}{{π}^{2}} {\sum }_{n = 1}^{∞} \frac{1}{{n}^{2}} {e}^{- {( \frac{π \cdot n}{L} )}^{2} D \cdot t} ( cos n π - 1 ) + \frac{1}{2} {C}_{0} \qquad ( 35 )$$

The value of nᵢ(t) we can define by solving Eq. (35).

Data for calculation of refractive index and diffusion coefficient of the agent can be taken from measurements of transmitted or back reflected intensity of non-polarized incident light for a particular wavelength. For that a computer program was designed which calculates the intensity of transmitted light as the sum of the directly transmitted light I_{T} and the scattered light I_{scα} using Mie theory for various values of refractive index of interstitial matter of skin in the range n=1.35-1.47.

$$\begin{matrix}{I}_{T} = {I}_{0} {e}^{- ρσ L} & ( 36 ) \\ {I}_{sca} = 2 {\sum }_{i = 1}^{12} I ( {ϕ}_{i} ) Δϕ & ( 37 ) \\ I = {I}_{T} + {I}_{sca} & ( 38 )\end{matrix}$$

where ρ is the amount of scatterers per unit area, σ is the effective cross-section of scattering, L is the sample thickness, φ is the angle of the detected scattered light, and Δφ is the angle of view of the detector.

The computer program selects corresponding refractive index of the base matter of skin by comparing calculated and experimental intensities with the independently measured sample extinction coefficient ρσ. Then the diffusion coefficient is calculated using Eq. (35).

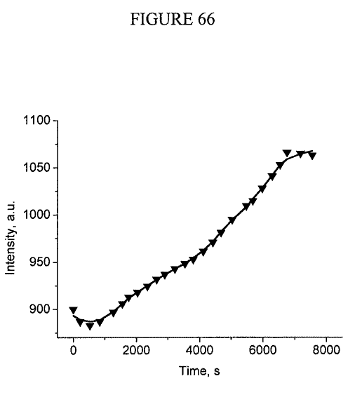

The data, presented in FIGS. 64 and 65, were calculated on the basis of experimental data for skin sample transmittance at 633 nm (FIG. 66) received for ex vivo pig skin sample treated using technology of the lattice of limited thermal damage islets in epidermis only (V. V. Tuchin, G. B. Altshuler, A. A. Gavrilova, A. B. Pravdin, D. Tabatadze, J. Childs, I. V. Yaroslavsky, Optical clearing of skin using flashlamp-induced enhancement of epidermal permeability, Lasers in Surgery and Medicine, vol. 38, issue 9, pp. 824-836, 2006). A special mask with dark spots was placed on the cleaned area, and skin in the projection of mask received by 2 flashlamp pulses of 27 J/cm², under the condition of intimate contact between skin, mask, and sapphire window of LuxRs handpiece. Experiments were carried out for a 40%-glycerol solution as a chemical agent applied after SC damaging.

The evaluated on the basis of experimental data in FIG. 66 mean value of diffusion coefficient of glycerol in the pig skin sample with perforated SC is equal to (0.84±0.08)×10⁻⁸ cm²/s, which is more than one order higher than these molecules diffusivity in intact SC, D_{α}=3×10⁻¹⁰ cm²/s, and is very close to their diffusivity in the intact living epidermis D_{α}=3×10⁻⁸ cm²/s (V. V. Tuchin, G. B. Altshuler, A. A. Gavrilova, A. B. Pravdin, D. Tabatadze, J. Childs, I. V. Yaroslavsky, Optical clearing of skin using flashlamp-induced enhancement of epidermal permeability, Lasers in Surgery and Medicine, vol. 38, issue 9, pp. 824-836, 2006).

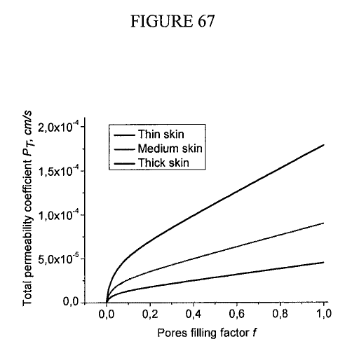

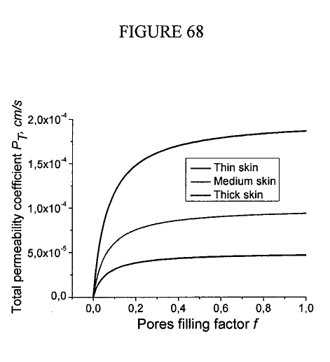

Results of modeling for the total permeability coefficient for a small molecular agent (filling) such as glycerol or glucose with the diffusivity for intact skin layers as D_{SC}⁰=3×10⁻¹⁰ cm²/s for stratum corneum, D_{E}⁰=3×10⁻⁸ cm²/s for epidermis, and D_{D}⁰=3×10⁻⁶ cm²/s for dermis are presented in Table D and FIGS. 67 and 68. The calculations were done using exact [Eq. (11)] and approximate [Eq. (15)] formulas. The diffusivity through laser drilled channels was taken equal to diffusion of the agent (filling) in water, D_{SC}^{p}=D_{E}^{p}=D_{D}^{p}=D_{W}^{a}=10⁻⁵ cm²/s. Drilled channel diameters (fill factors) were supposed to be equal for each layer. Calculations were done for different values of fill factor in the wide range attainable in experiment for three skin models with different thicknesses: thin, medium and thick.

TABLE D
Calculations of the total permeability coefficient for an agent
(filling) with the diffusivity for intact skin layers equal to
D_{SC}⁰ = 3 × 10⁻¹⁰ cm²/s, D_{E}⁰ = 3 × 10⁻⁸ cm²/s,
D_{D}⁰ = 3 × 10⁻⁶ cm²/s and for laser drilled channels as for
diffusion of the agent (filling) in water D_{SC}^{p} = D_{E}^{p} = D_{D}^{p} ≡
D_{W}^{a} = 10⁻⁵ cm²/s for models of skin with different thicknesses:
thin, medium and thick

| | The total permeability coefficient P_{T} (×10⁵) | | | | | |
|---|---|---|---|---|---|---|
| | Thin skin | | Medium skin | | Thick skin | |
| | h_{SC} = 0.001 cm | | h_{SC} = 0.0015 cm | | h_{SC} = 0.002 cm | |
| | h_{E} = 0.005 cm | | h_{E} = 0.01 cm | | h_{E} = 0.02 cm | |
| Pores filling | h_{D} = 0.05 cm | | h_{D} = 0.1 cm | | h_{D} = 0.2 cm | |
| factor f | Eq. (11) | Eq. (15) | Eq. (11) | Eq. (15) | Eq. (11) | Eq. (15) |
| 0 | 0.03 | 0 | 0.02 | 0 | 0.01 | 0 |
| 0.01 | 1.54 | 2.50 | 0.80 | 1.33 | 0.42 | 0.71 |
| 0.05 | 3.79 | 8.33 | 1.93 | 4.35 | 0.98 | 2.27 |
| 0.1 | 5.16 | 11.76 | 2.61 | 6.06 | 1.32 | 3.12 |
| 0.2 | 6.98 | 14.81 | 3.52 | 7.55 | 1.77 | 3.85 |
| 0.3 | 8.48 | 16.22 | 4.27 | 8.22 | 2.15 | 4.17 |
| 0.4 | 9.89 | 17.02 | 4.97 | 8.60 | 2.50 | 4.35 |
| 0.5 | 11.25 | 17.54 | 5.66 | 8.85 | 2.84 | 4.46 |
| 0.6 | 12.59 | 17.91 | 6.33 | 9.02 | 3.18 | 4.54 |
| 0.7 | 13.92 | 18.18 | 6.99 | 9.15 | 3.51 | 4.61 |
| 0.8 | 15.24 | 18.39 | 7.65 | 9.25 | 3.85 | 4.65 |
| 0.9 | 16.55 | 18.56 | 8.31 | 9.33 | 4.18 | 4.69 |
| 1 | 17.86 | 18.69 | 8.97 | 9.39 | 4.50 | 4.72 |

It follows from Table D and FIGS. 67 and 68 that approximate Eq. (15) predicts rather well total permeability of low molecule agent through different skin models for high and very small filling factors of laser drilled channels. The total permeability significantly increases for drilled skin in comparison with intact in 54-fold for thin skin, 43-fold for medium thickness skin, and 31 for thick skin models when filling factor changes from 0 to 0.01. Because dermal thickness dominates for all skin models and agent's diffusivity in intact dermis is only one order less than in water the total permeability increases approximately equally, 10.7-11.5-fold, with filling factor increase from 0.01 to 1 for different skin models.

E. Delivery of Chromophores

As noted above, the micro-holes can be used to deliver a chromophore into tissue. Subsequently, the chromophore is selectively heated using EMR or other energy. As a result, the tissue, organ, gland or other structure adjacent to the chromophore can be ablated, damaged or otherwise altered. Use of chromophores delivered through micro-holes may have several advantages over selective photothermolysis at it is presently practiced or other present treatments and methods. For example, by delivering a chromophore into the tissue, a chromophore that has a very high contrast with the surrounding tissue can be chosen, such that the chromophore absorbs EMR at a given wavelength far more readily than a chromophore that may already be present in the tissue. Thus, the chromophore will require much less energy to absorb the same amount of heat as, for example, a naturally occurring chromophore. Therefore, less energy will be required to achieve the same result. Thus, the treatment may be less painful, and may be capable of being performed without cooling. The contrast between the applied chromophore and the tissue can be further accentuated by first increasing the translucence of the tissue by infusing a substance such as glycerol into the micro-holes (or into a different set of micro-holes). Generally, a higher contrast in the degree of absorption of energy at a given wavelength or wavelengths by the chromophore as compared to the treated tissue, will allow the tissue to be successfully treated using relatively less energy.

In other embodiments, several different chromophores could be applied after the creation of a set of micro-holes. If each chromophore had complimentary coefficients of absorption and/or were preferentially absorbed (or not absorbed) by various tissues, a first chromophore could be irradiated with a wavelength(s) of EMR that was not readily absorbed by the second chromophore and that did not disturb the second chromophore. Thus, several successive treatments could be performed without the need to retreat the tissue to create a new set of micro-holes to introduce the second chromophore at a later time. Similarly, in some embodiments, two different tissue, tissue structures or tissue organs could be treated using different chromophores. Many other various of such types of treatments are possible.

By way of example, chromophores can be introduced into skin tissue to treat sebaceous glands (e.g., to treat acne) or to treat subcutaneous tissue (e.g., for fat reduction or to treat cellulite). In the case of acne, micro-holes can be formed to a depth of approximately 0.5-1.0 mm in the surface of affected skin tissue. A chromophore (e.g., carbon particles, can be placed in the micro-holes. Subsequently, EMR is applied to the chromophore. It is preferable, but not essential, that the EMR have a wavelength corresponding approximately to a high or maximum coefficient of absorption of the chromophore and a low or minimum coefficient of absorption of the surrounding tissue. For example, EMR having one or more wavelengths in the range of 800 nm-1200 nm could be used.

To treat subcutaneous tissue, micro-holes can be formed to a depth of approximately 3.0 mm from the surface and into the affected tissue. Referring to FIG. 32, micro-holes 1060 are formed in from a surface of a tissue volume 1062. The micro-holes 1060 extend through an epidermal layer 1064 and a dermal layer 1066 and into a subcutaneous fat layer 1068 of tissue volume 1062. A chromophore 1070 (e.g., carbon particles) can be placed in the micro-holes. The micro-holes can deliver the chromophore to the fat tissue, some of which then exits the micro-hole and spreads through the fat tissue. Subsequently, EMR can be applied to the chromophore. Again, it is preferable, but not essential, that the EMR have a wavelength corresponding approximately to a high or maximum coefficient of absorption of the chromophore and a low or minimum coefficient of absorption of the surrounding tissue. For example, EMR having one or more wavelengths in the range of 800 nm-1200 nm could be used.

In other embodiments, a chromophore can be delivered throughout an area of the dermis for a particular treatment, for example, hair removal or permanent hair reduction by delivering energy to the chromophore to destroy or impair the function of a hair follicle. Alternatively, the chromophore could be delivered locally within the dermis to treat a particular volume or structure. For example, micro-holes could be created in the area of a pigmented or vascular lesion to the depth of the lesion, preferably to the depth of the lower boundary of the lesion. A chromophore such as carbon can then be delivered through the holes. The chromophore can remain within the holes or, in other embodiments, the chromophore (or a composition containing the chromophore) could be allowed to diffuse into the lesion or other structure being treated. Subsequently, the volume of tissue containing the chromophore is irradiated to heat the chromophore and cause localized tissue damage to the lesion, thereby removing the lesion during the healing process.

The above descriptions are exemplary only. Many other embodiments are possible.

F. Delivery of Fillers and Non-Drugs

In addition to drugs, micro-islets and other micro-structures can be used to deliver bio-inert materials such as fillers. For example, micro-holes, micro-cavities, micro-grooves and other micro-structures can be used to apply a filler to, for example, alter a physical or mechanical property of the skin.

By controlling the depth and fill factor of the micro-holes, micro-channels or other micro-structures, the fillers and other bio-inert materials can be introduced evenly across and throughout the skin tissue as desired. Such procedures allow for the delivery of substances to precise depths, which are not typically possible using other methods, such as delivery of substances with needles. Such fillers can, for example, be applied using pressure applied from a gun or other handpiece to saturate the treated columns or fill any holes or pits.

Substances that are not readily absorbed into the body and/or that are not metabolized or eliminated can also be used. Examples of such substances include tattoo ink, cosmetics, and substances capable of providing ultraviolet (“UV”) protection. Such substances may remain embedded indefinitely and, therefore, provide essentially a permanent or semi-permanent tattoo, cosmetic or UV protection. Other permanent, semi-permanent or temporary substances can be embedded in the micro-holes. Such fillers can further include organic materials such as fat. Exemplary substances that can be used include titanium oxide, aluminum oxide (sapphire), silicon oxide, diamond, quartz, silica, zirconium oxide, hydroxylanitite, apatite, silver, gold, polymethyl methacrylate, other acrylics, other glasses, carbon black, magnetic nanoparticles, nanoshells, fullerens, astrolens, porous silicon, and hyaluronic acid fillers (such as Perlane and Restylane) can be used to alter the optical and mechanical properties of the skin. Many other substances are possible.

Fillers can be used to change the appearance of the skin and to increase scattering or absorption properties, for example to alter the skin's luminescence and reflectance. Fillers can also be used to alter the elasticity and tightness of the skin and can be used to plump certain tissues. In some embodiments, particles or compositions of particles having a refractive index of between 1.5 and 3.0 can be delivered into skin tissue to alter the optical properties of the skin. For example, sapphire has an index of refraction of approximately 2.4 which is much higher than that of skin. Thus, sapphire may be used cosmetically to alter the overall refraction of skin tissue. Additionally, skin whitening and volumetric brightening (improvement of skin albedo) can be achieved by delivering substances having a relatively high refractive index.

In another embodiment, referring to FIG. 33, fillers can be used to increase scattering in the surrounding tissue to block an existing tattoo. For example, micro-holes 1080 are formed in skin tissue 1082. The micro-holes have a width of approximately 100 μm, a extending into the dermis 1084 but above the depth of the tattoo 1086, which is typically 1/64 to 1/16 of an inch in depth, and a fill factor of 30-70%. A filler 1088 is chosen to increase the scattering properties of the skin to obscure the tattoo. The filler chosen will depend on the optical characteristics of the tattoo, primarily the color. Although this method can be used obscure an existing tattoo completely, it may be particularly useful in obscuring the remnants of a tattoo that has been treated to remove it due to the resistant nature of some colors of tattoo inks to removal using current techniques. It may be preferable to provide an even distribution of micro-islets to ensure even application of a filler or other substance.

Similar principles can be applied to block other visible structures in the skin, such as lesions or variations in skin tone. Generally, by increasing the scattering, reflectance, and/or the fluorescence of the tissue, the radiancy of the tissue will be increased and structures in the tissue can be obscured and/or smoothed. Conversely, decreasing the scattering, reflectance, and/or the fluorescence can cause the tissue to become more translucent. In the later case, skin that is more translucent and/or transmissive to some or all wavelengths of EMR can be useful for diagnostic purposes. For example, by greatly reducing the scattering of the tissue, the tissue may be imaged or EMR can otherwise be applied for diagnostic purposes and much deeper layers of tissue can be effectively accessed for such purposes.

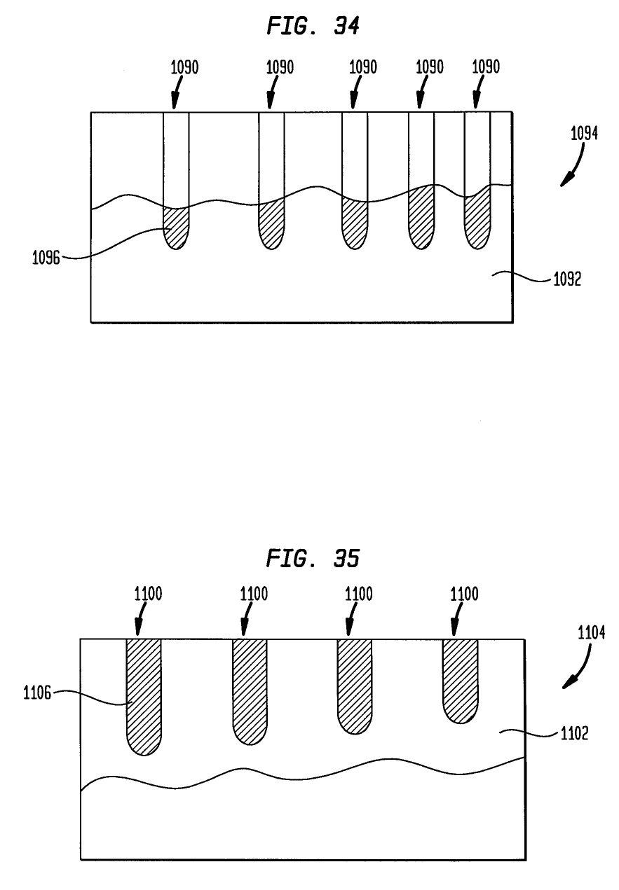

Micro-islets can be used to deliver a permanent or semi-permanent sunscreen. For example, referring to FIG. 34, an array of micro-holes 1090 can be used to create a permanent sunscreen in a dermis layer 1092 of skin tissue 1094. The micro-holes 1090 are approximately 100 um in diameter, extend into the upper layers of the dermis (approximately 0.4 μm, but dependent on the thickness of the epidermis), and have a fill factor of approximately 40%. The micro-holes are filled with a filler 1096, in this case zinc oxide or titanium oxide, to provide protection from ultraviolet radiation.

Referring to FIG. 35, an alternate embodiment provides for semi-permanent protection from ultraviolet radiation. An array of micro-holes 1100 can be used to create a semi-permanent sunscreen in a epidermis layer 1102 of skin tissue 1104. The micro-holes AA are approximately 100 um in diameter, extend into the upper layers of the dermis (approximately 0.1-0.4 μm, but dependent on the thickness of the epidermis), and have a fill factor of approximately 40%. The micro-holes also are filled with a filler 1106, in this case zinc oxide or titanium oxide, to provide protection from ultraviolet radiation. However, because the epidermis is continually regenerated and old layers are replaced over time, any filler delivered to the epidermis will not remain their permanently. However, because the filler is applied near the surface, the method provides a degree of protection over a long period of time without requiring the continued reapplication of a topical substance. Thus, the method is particularly useful for treating sun-exposed portions of the body prior to a vacation or other trip or seasonal coverage or extended but limited period of sun-exposure.

The permanent and semi-permanent sunscreens are not applied to the entire area of the skin tissue, and thus, do not provide complete protection to the tissue. However, the protection may be superior to currently used topical lotions and sprays due to the very thin layers of protection (several microns in thickness) provided by topical sunscreens. Furthermore, topically applied sunscreens may not adequately protect skin tissue due to 1) reduction of light scattering in the stratum corneum due to optical immersion and 2) inhomogeneous distribution of the topically applied substances. (See J. Lademann, A. Rudolph, U. Jacobi, H.-J. Weigmann, H. Schaefer, W. Sterry, and M. Meinke “Influence of Nonhomogeneous Distribution of Topically Applied UV Filters on Sun Protection Factors,” J. Biomed. Opt., vol. 9, 2004, pp. 1358-1362). Both effects lead to reduction in the efficacy of topical sunscreen, because there are fewer interactions of migrating photons in skin with sunscreen material when there is less scattering, and also because areas free or nearly free of sunscreen do not block ultraviolet radiation.

Thus, although the protection is not completely applied across the entire skin surface, it provides an added degree of protection that may be superior to topicals, due to, among other things, the increase in scattering that promotes absorption by the sunscreen filler material. Additionally, these methods may be combined with standard application of topicals, to provide even greater protection, while still providing protection when a topical has not been applied.

In other embodiments, nanoparticles can be delivered into the tissue to allow the particles to be used within the tissue. The nanoparticles can be tuned to be responsive to particular wavelengths.

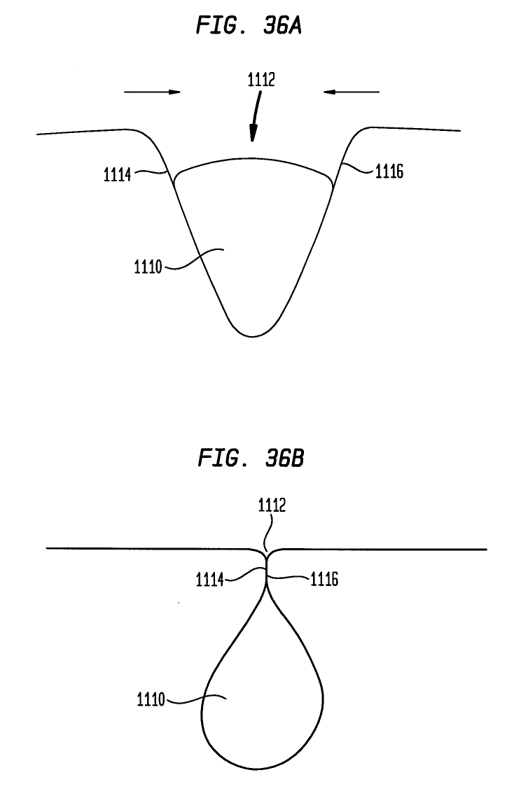

Referring to FIGS. 36A and 36B, a filler 1110 can be delivered into a micro-groove 1112 to provide desired optical and/or mechanical properties in the tissue being treated. When the walls 1114 and 1116 of microgroove 1112 are pressed together, they encompass the filler 1110, which retains a significant portion of the ablated volume while still reducing the skin area. Thus, the tissue is tightened but remains plump.

The substances that are delivered can be used for skin rejuvenation, hydration and similar treatments. For example, delivery of antioxidant preparations (alfa-hydroxy acids) that leads to additional skin hydration can provide enhanced dead keratinocyte exfoliation, and, thus, to improvement of mechanical properties of skin (elasticity and softness) and smooth profile. Similarly, cosmetic hydration fillings for keratin and collagen hydration can improve mechanical properties of skin (elasticity and softness). Macromolecular fillings (e.g., collagen, elastin, protoglycans, and etc.) can also improve mechanical properties of skin (elasticity and softness).

In other embodiments, other substances can be applied for different purposes. For example, skin color can be improved by delivering skin lightening complexes, such as Bright Idea™ Artistry lightening complex. Collagen growth can be stimulated using internal cosmetics such as Rejuva™. Skin moisture and elasticity can be enhanced by delivering chondrotin sulfate to maintain skin moisture and elasticity.

An ink can be delivered to form a permanent or temporary tattoo. The lattices can also be created to control tattooing of tissue. For examples, holes bearing different color pigments can be created. Similarly, a reversible tattoo can be created using magnetized particles is possible.

Another application of laser skin perforation (drilling) is stem cells stimulation and delivery for the cosmetics purposes and skin disease treatment. According to the most generally accepted definition, stem cells are a special group of undifferentiated cells possessing two fundamental properties: they are capable of self-maintenance and differentiation into specialized tissue-forming cells. According to their origin, stem cells are divided into embryonic and somatic ones.

Successful long-term restoration of continuously self-renewing of the skin depends on the use of extensively self-renewing stem cells (Laura Alonso and Elaine Fuchs, “Stem cells of the skin epithelium,” PNAS, vol. 100, Suppl. 1, pp. 11830-11835, 2003). Tissue stem cells form the cellular base for organ homeostasis and repair. Stem cells have the unusual ability to renew themselves over the lifetime of the organ while producing daughter cells that differentiate into one or multiple lineages. The mammalian skin epithelium exhibits remarkable turnover, punctuated by periods of even more rapid production after injury due to burn or wounding.

Perhaps the most universally accepted criteria for keratinocyte stem cells are that they are normally slow-cycling (or, perhaps more accurately, rarely cycling) in vivo; that they can self-renew and are responsible for the long-term maintenance of the tissue (see FIGS. 26-28); that they can be activated by injuring to proliferate and to regenerate the tissue; and that they have a high proliferative potential. Injure healing involves local cutaneous cells for reconstituting the epidermis at the same time distant bone marrow-derived cells (circulating in blood) and the adjacent uninjured dermal mesenchymal cells take part in reconstituting the dermal fibroblast population (Fathke C., Wilson L., Hutter J., Kapoor V., Smith A., Hocking A. & Isik F., “Contribution of bone marrow-derived cells to skin: collagen deposition and wound repair,” Stem Cells 22, pp. 812-822, 2004).

Therefore, laser skin perforation (drilling) that provides in depth controllable injure of the skin may stimulate stem cells to activate processes of skin rejuvenation due to reconstituting of the epidermis by activating of local cutaneous cells as well as reconstituting the dermal collagen and fibroblast population by activating of distant bone marrow-derived cells (circulating in blood) and dermal mesenchymal cells.

One significant aspect of epidermal differentiation is the regulation of lineage commitment and switching of differentiation lineages of epidermal stem cells between hair follicles, an adipose tissue, sebaceous glands and interfollicular epidermis, that provides the prospect of engineering the epidermis to produce more hair follicles for balding men, to banish excess sebum production for teenagers and to ensure smooth, hair-free legs for women.

Several types of somatic stem cells have the remarkable capacity to differentiate into other types of tissues. Stem cells from the skin have the capacity to form multiple cell lineages during development. Furthermore, the injected stem cells altered their epidermal phenotype and expressed the appropriate proteins for the tissues into which they developed, demonstrating that somatic epidermal stem cells have the ability to produce cells of different lineages during development. These data suggest that somatic epidermal stem cells may show a generalized plasticity expected only of embryonic stem cells and that environmental (extrinsic) factors may influence the lineage pathway for somatic stem cells. Thus, the skin could be a source of easily accessible stem cells that are able to be reprogrammed to form multiple cell lineages (Liang L. & Bickenbach J. R., “Somatic epidermal stem cells can produce multiple cell lineages during development,” Stem Cells 20, pp. 21-31, 2002).

The multipotent precursor cells can be isolated from mammalian dermis (Toma J. G., Akhavan M., Fernandes K. J., Barnabe-Heider F., Sadikot A., Kaplan D. R. & Miller F. D., “Isolation of multipotent adult stem cells from the dermis of mammalian skin,” Nat Cell Biol 3, pp. 778-784, 2001) and neonatal human foreskin tissue (Toma J. G., McKenzie I. A., Bagli D. & Miller F. D., “Isolation and characterization of multipotent skin-derived precursors from human skin,” Stem Cells 23, pp. 727-737, 2005). These cells were expanded, characterized, and termed as skin-derived precursors (SKPs). The SKPs can differentiate into both neural and mesodermal progeny. Human SKPs grew in suspension as spheres in the presence of the mitogens fibroblast growth factor 2 and epidermal growth factor and expressed nestin (a type VI intermediate filament (IF) protein), fibronectin (a high-molecular-weight glycoprotein containing about 5% carbohydrate that binds to receptor proteins that span the cell's membrane, called integrins), vimentin (a member of the IF family of proteins, IF are an important structural feature of eukaryotic cells, they, along with microtubules and actin microfilaments, make up the cytoskeleton), and characteristic embryonic transcription factors. Human SKPs could be maintained in culture for long periods of time and would still differentiate into neurons and smooth muscle cells, including cells with the phenotype of peripheral neurons and Schwann cells (a variety of neuroglia that mainly provide myelin insulation to axons in the peripheral nervous system). SKPs are an endogenous multipotent precursor cell present in human skin that can be isolated and expanded and differentiate into both neural and mesodermal cell types (Toma J. G., McKenzie I. A., Bagli D. & Miller F. D., “Isolation and characterization of multipotent skin-derived precursors from human skin,” Stem Cells 23, pp. 727-737, 2005; Fernandes K. J., McKenzie I. A., Mill P., Smith K. M., Akhavan M., Bamabe-Heider F., Biernaskie J., Junek A., Kobayashi N. R., Toma J. G., Kaplan D. R., Labosky P. A., Rafuse V., Hui C. C. & Miller F. D., “A dermal niche for multipotent adult skin-derived precursor cells,” Nat Cell Biol 6, pp. 1082-1093, 2004).

Multipotent precursor cells isolated from mammalian dermis can be used as exogenous stem cells that being inserted into aged or pathological skin may differentiate into smooth muscle cells (and other types of mesodermal cells) and can improve peripheral nervous system, and finally can lead to skin rejuvenation. Skin porosity produced by laser can be considered as a prospective tool for exogenous stem cell implanting. Laser drilled holes (caverns) in the skin and underlying tissue can serve as an excellent natural bioreactor, where tissue environment works as a natural biochemical infrastructure that, of course, if needed can be modified (modulated) by inserting accompany biochemical ingredients via the same drilled holes. In such natural bioreactor besides biochemical environment, other aspects of cell division and modification, such as cell adhesion, tension, and sterile conditions can be realized automatically.

Most cells in tissues contact an extracellular matrix on at least one surface. These complex mixtures of interacting proteins provide structural support and biological signals that regulate cell differentiation and may affect stem cell differentiation. Individual components of the extracellular matrix, such as laminin-1 (laminins are the major non-collagenous component of the basal lamina, such as those on which cells of an epithelium sit; they are a family of glycoproteins that are an integral part of the structural scaffolding of basement membranes in almost every animal tissue; laminins are secreted and incorporated into cell-associated extracellular matrices) or collagen I (a matrix protein that is a widely distributed high molecular weight glycoprotein found in most extracellular matrices and blood), do not influence the growth or morphology of the cells. In contrast, a basement membrane extract, Matrigel (the trade name for a gelatinous protein mixture secreted by mouse tumor cells and marketed by BD Biosciences, this mixture resembles the complex extracellular environment found in many tissues and is used by cell biologists as a substrate for cell culture), containing multiple extracellular matrix components, induces the cells within 4 days to form immature glandular- and tubular-like structures, many of which contain a lumen with polarized epithelium and microvilli. Such structures were seen in vitro when the cells were grown in the bioreactor and when the cells were injected into mice. These tubular- and glandular-like structures were polarized epithelia based on immunostaining for laminin and cytokeratin (Philp D., Chen S. S., Fitzgerald W., Orenstein J., Margolis L. & Kleinman H. K., “Complex extracellular matrices promote tissue-specific stem cell differentiation,” Stem Cells 23, pp. 288-296, 2005).

Typically in tissue engineering the following parameters are under monitoring and control: cell behavior on the scaffolds, cell proliferation, location, phenotype, and activity, scaffold degradation and matrix production, tissue formation and characterization of growth, implant behaviour in the patient (Alicia El Haj, “Stem cell therapy: Challenges for growth control and delivery,” SPIE Photonics West Symposium 2007). Typical scaffold based on poly-L-lactide acid (PLLA) has a 90%-porosity and 300 μm-pores size (see FIG. 29). Channels in porous chitosan scaffold are well seen in FIG. 30. Chitosan is a linear polysaccharide composed of randomly distributed β-(1-4)-linked D-glucosamine (deacetylated unit) and N-acetyl-D-glucosamine (acetylated unit). It has a number of commercial and possible biomedical uses. Chitosan is produced commercially by deacetylation of chitin, which is the structural element in the exoskeleton of crustaceans (crabs, shrimp, etc.).

Parameters for efficient tissue engineering, such as porosity, channels diameter and depth, can be easily provided in in vivo tissue using laser drilling technique. Porosity typically will change with cell seeding and tissue turnover.

Such multipotent stem cells as epithelial/mesenchymal cells when seeded into porosity scaffold may produce a skin equivalent, in particular, dermal mesenchymal cells take part in reconstituting the dermal fibroblast population (Fathke C., Wilson L., Hutter J., Kapoor V., Smith A., Hocking A. & Isik F., “Contribution of bone marrow-derived cells to skin: collagen deposition and wound repair,” Stem Cells 22, pp. 812-822, 2004).

Chemical stimulators, e.g. growth factor, agonist (a substance that binds to a specific receptor and triggers a response in the cell, it mimics the action of an endogenous ligand (such as hormone or neurotransmitter) that binds to the same receptor) provide controllable cell growing and tissue rejuvenation. Laser drilling technology can be useful in supplying (perfusion) of such chemical stimulators and other biochemical components if needed.

Mechanical compression and seeded cell adhesion for tissue matrix and other tissue components production can be provided within laser drilled channels due to skin and subcutaneous tissue intrinsic tension, as well as can be easily provided externally in any direction.

Methods of physical influence on stem cells behavior, such as cyclic compressive loading of bone-marrow mesenchymal stem cells (Huang C. Y., Hagar K. L., Frost L. E., Sun Y. & Cheung H. S., “Effects of cyclic compressive loading on chondrogenesis of rabbit bone-marrow derived mesenchymal stem cells,” Stem Cells 22, pp. 313-323, 2004; Huang C. Y., Reuben P. M. & Cheung H. S., “Temporal expression patterns and corresponding protein inductions of early responsive genes in rabbit bone marrow-derived mesenchymal stem cells under cyclic compressive loading,” Stem Cells 23, pp. 1113-1121, 2005)), cell orientation, adhesion, and migration induced by exogenous electrical stimulus (Sun S. & Cho M., “Human fibroblast migration in three-dimensional collagen gel in response to noninvasive electrical stimulus. II. Identification of electrocoupling molecular mechanisms,” Tissue Eng 10, pp. 1558-1565, 2004; Sun S., Wise J. & Cho M., “Human fibroblast migration in three-dimensional collagen gel in response to noninvasive electrical stimulus. I. Characterization of induced three-dimensional cell movement,” Tissue Eng 10, pp. 1548-1557, 2004) can have their reliability on the level of biochemical ones.

Therefore, electrophoresis, sonophoresis, and magnetophoresis techniques can be useful in in vivo tissue engineering of laser drilled skin. In particular, magnetic tagging of stem cells delivery, cell guiding to sites of repair and switch on/of their activation can be used. In vitro transduction of magnetic particles into C12S cells encoding GFP in the presence of a magnetic field was shown. Magnets implanted in a dermal wound nude mouse model (Luciferase reporter system) is also reported. The mother and daughter cells are dipole cells (sources of electric field). If the disposition of mother cells is unidirectional and they predominate over daughter cells, the latter cells transform into other cells, i.e. are differentiated (Yavisheva T. M., Shcherbakov S. D., Golubeva I. S., Sharafutdinov G. Z. & Savluchinskaya L. A., “Structural organization of the basal layer and morphofunctional characteristics of mouse epidermal cambial cells,” Bull Exp Biol Med 137, pp. 513-516, 2004).

G. Absorption and Delivery of Drugs

As in the embodiments that create micro-holes in nail tissue, micro-holes can similarly be used to facilitate the delivery of drugs or other substances through the skin or other soft tissues. For example, a mixture containing a drug (or drugs) and/or other substances having low absorption rates can be applied to the surface of the skin in an area that has been treated with EMR to create and array of micro-holes. Treatments according to this embodiment may involve treatments of one or more different anatomical sites of human body, such arms, legs, forehead, axilla, etc., and multiple target sites or tissue types can be treated simultaneously.

Presently, many potential therapies for the treatment of skin or some other superficial organ diseases are declined due to the toxic effect of drugs taken orally, by injection or intravenously. Similarly, many approved painkillers are also taken orally, by injection, intravenously, or superficially on a daily (or even hourly) basis for the treatment of skin or other superficial organ pain. Applying the treatment substance having a low dissolving rate inside a human body has been successfully used for the treatment of long lasting pain or for preventative purposes. In most such cases, the substance is a matrix of tablets, which dissolve slowly and release embedded medicine to maintain the necessary concentration locally.

Such treatment substances having a low dissolving rate can be applied to micro-holes, such as micro channels, for the treatment of human skin and other diseases. The uptake of the treatment substance can be enhanced by embedding the substance within the micro-holes using chemical enhancers (e.g., polar solvents (such as decylmethylsulfoxide) and polyenic antibiotics (to enhance membrane permeability), mechanical or other energy, for example, positive and/or negative pressure, magnetic fields applied to magnetic substances, electric fields applied to electrically charged substances (e.g., iontophoresis), local skin heating, massage or other mechanical manipulation of the tissue, sprays (e.g., high pressure sprays with small droplets) light waves or other EMR-induced stress, acoustic waves including sonophoresis and other forms of ultrasound. The treatment method may involve (but would not necessarily be limited to) one or more steps of treatment with single wavelengths, and may also be applied in the course of two or more repetitions of the treatment procedure in one or more treatment sessions. Multiple wavelengths may also be used, depending on the application, which may be applied using the same or different light sources.

Many substances can be used, including, for example, pure substances, mixtures containing one or more active compounds; and compounds in an active or inactive matrix. The substance applied can be in various forms, including, without limitation, liquid, solid, gel or aerosol forms.

Drugs or other substances having high absorption rates can also be applied, but the mechanism is presently thought to work more beneficially with drugs having a low absorption rate. Furthermore, in other embodiments, a substance normally having a high dissolving rate can be applied slowly, because the dissolving rate can be dictated by the active ingredients and/or inactive ingredients. Thus, a mixture having a low dissolving rate can be manufactured to include an ingredient that normally has a high dissolving rate.

In some embodiments, the treatments involve three steps. First, micro-holes are created in the tissue, such as human skin. The micro-holes are created at the selected anatomical location using a device similar to device 500 as shown in FIGS. 7-9.

Second, the substance is embedded in the micro-holes. This step can be performed by various methods, including, without limitation, simple diffusion, vesicle/particle transporters, physical mechanisms, chemicals, or electrical mechanisms, electroporation, iontoporation, sonophoresis, magnetophoresis, photomechanical waves, niosomes, and transfersomes.

Third, the substance is sealed within the micro hole. This can be accomplished by various methods, including, without limitation, natural healing, healing creams, covering with, e.g., tapes or strips, and sutures.

The process may need to be repeated several times depending on the application.

Many embodiments are possible, including variations of parameters used. In some embodiments, the drugs can be encapsulated. For example, the drug can be encapsulated in borate-phosphate glass soluble in tissue, liposomes, magnetic particles, polymer, or biodegradable polymer. Furthermore, the substance embedded in the micro hole need not be a drug.

To examine the efficacy of using micro-holes to embed substances within tissue, several experiments were performed which demonstrate the successful application of substances into micro-holes in tissue.

1. Experiment 1—Creating an Open Hole in the Human Skin In Vivo

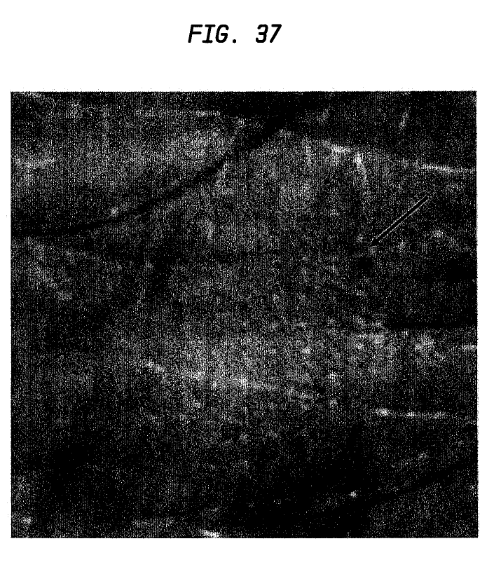

Less energy is required to create micro-holes in the human skin than is required to make similarly sized micro-holes in nail tissue. Approximately, 5 mj per beam is enough energy to make holes traversing through the epidermis. In this case, the treatment was performed on the subject's right arm in vivo. The treatment parameters were: a wavelength of 2940 nm at 5.5 milijoules per beam, using a single pulse of 200 microseconds. As a result of the treatment, the subject experienced a similar sensation after applying a 10% ammonia solution as that described in conjunction with Experiment 4 above. The burning sensation indicates that hole went through the stratum corneum. Referring to FIG. 37, the appearance of blood was observed (delineated by an arrow in FIG. 37) in some micro-holes, which indicates that the hole went through at least the epidermis.

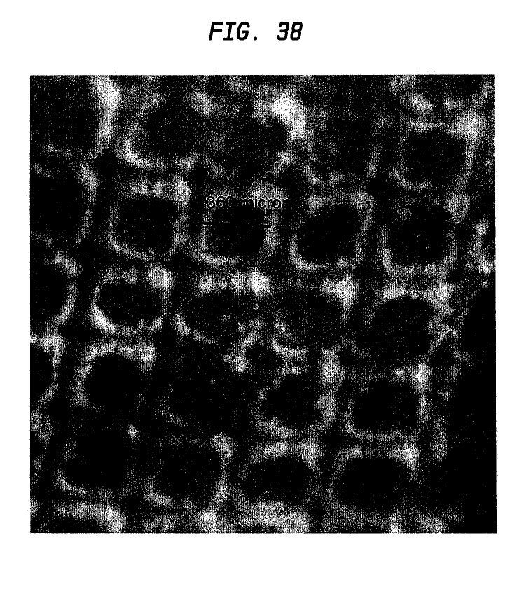

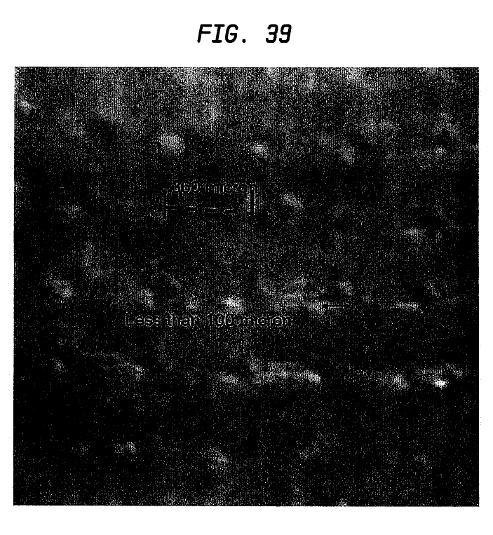

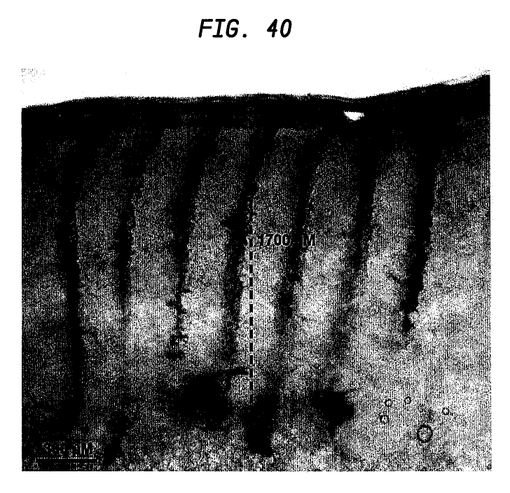

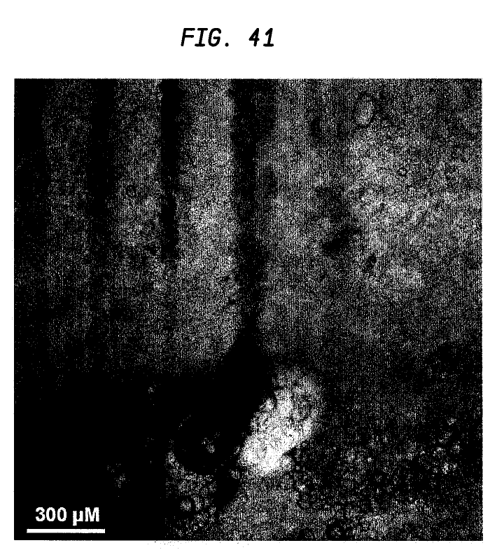

FIGS. 38 and 39 are close up views of the micro-holes of FIG. 37. FIG. 38 shows the treatment area prior to washing. FIG. 39 shows the treatment area after washing. The pitch of the micro-holes was 360 micrometers. The diameter of the micro-holes is less than approximately 100 microns.

2. Experiment 2—Treatment of Tissue Using 2940 nm and a Pitch of 330 μm

In the following experiment, a sample of Yucatan black pig skin was treated in vitro using a device similar to device 500 of FIGS. 7-9. The device applied EMR at a wavelength of 2940 nm using a pitch of 30 micrometers to form the EMR islets. The skin was stored at −20C for approximately 3 months. The skin was defrosted prior to testing and warmed to room temperature. The skin was marked with a marking pen and treated with the EMR. The skin was then stretched and pinned down on a flat surface. A drop of black tattoo ink was placed on the treated area and massaged into the micro-holes. (In another test, red organic molecules in water (Eosin) were applied to the micro-holes in a method similar to the procedure described for tattoo ink.) The skin was released, and a 6 mm biopsy was obtained from the treated area. The biopsy was frozen and manually cut into 100-300 micron sections. The segments were examined with a BH2 light microscope (Olympus) using a CoolPix-8400 photo camera (Nikon).

The treatment parameters are shown in Table E.

TABLE E

| | | Energy per | # of | Samples | Temperature |
|---|---|---|---|---|---|
| # | Screen of tip | MB, mJ | pulse | collected | of skin, ° C. |
| 1 | with lenses | 1.5 | 1 | 4 to 6 | RT |
| 2 | | 3 | | samples | |
| 3 | | 6 | | for | |
| 4 | | 9 | | formalin | |
| 5 | | 12 | | | |

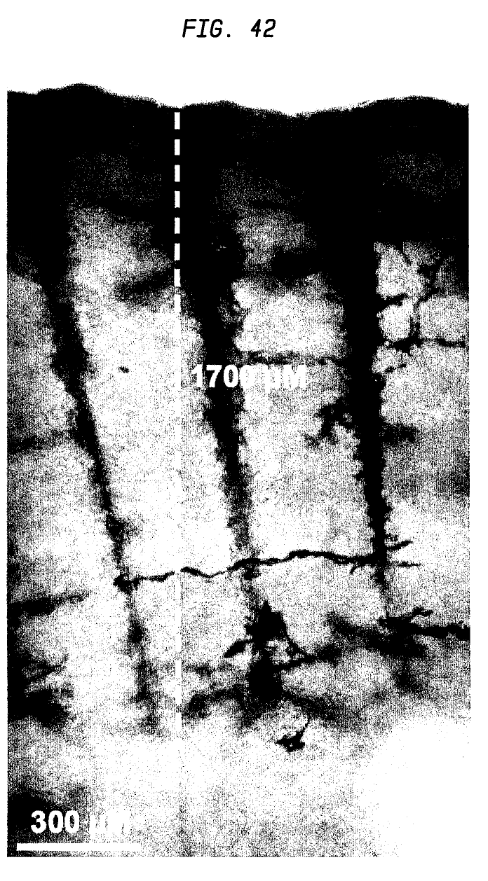

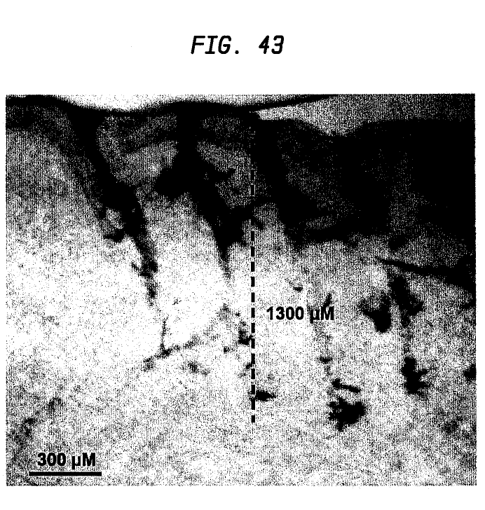

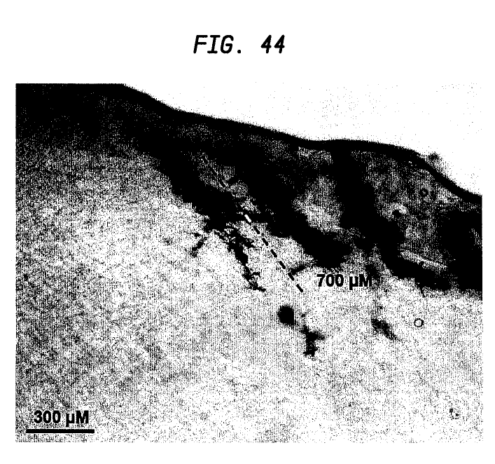

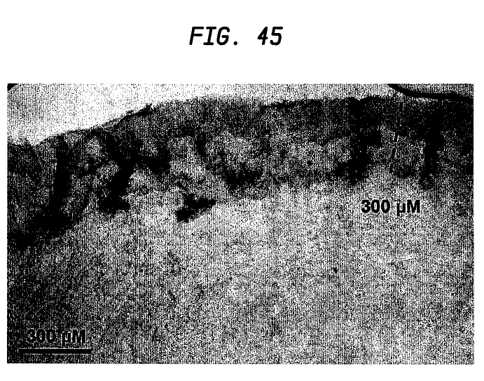

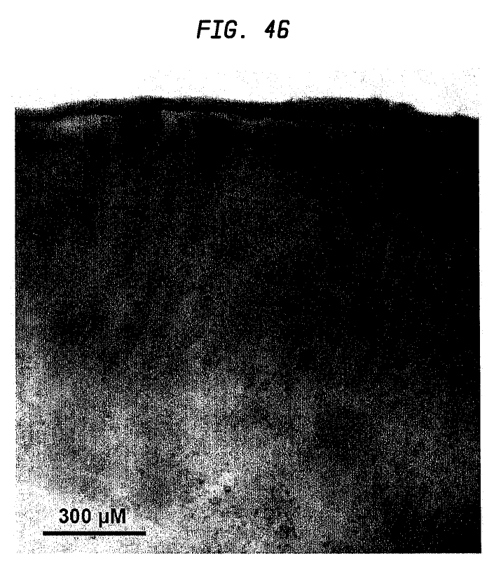

The results of the experiment are shown in FIGS. 40-47. Referring to FIG. 40, the skin was treated with one pulse of approximately 12 mj per beam. The micro-holes traverse through the epidermis and dermis. Referring to FIG. 41, the skin was treated with one pulse of approximately 12 mj per beam. Leakage of ink is observed in the hypodermis through the MC traverses through the epidermis and dermis. Referring to FIG. 42, the skin was treated with approximately 9 mj per beam. The micro-holes traverse through the epidermis and dermis. Referring to FIG. 43, the skin was treated with one pulse of approximately 6 mj per beam. The micro-holes traverse through the epidermis and larger part of dermis. Referring to FIG. 44, a section of pig skin was treated using a device similar to device 500, applying a wavelength of 2940 nm to the tissue in vitro. The treatment parameters were: one pulse of approximately 3 mj per beam. The micro-holes traverse through the epidermis and partially into the dermis. Referring to FIG. 45, a section of pig skin was treated using a device similar to device 500, applying a wavelength of 2940 nm to the tissue in vitro. The treatment parameters were: one pulse of approximately 1.5 mj per beam. The micro-holes traverse through the epidermis and papillary dermis. Referring to FIG. 46, a section of pig skin was treated using a device similar to device 500, applying a wavelength of 2940 nm to the tissue in vitro. The treatment parameters were: one pulse of approximately 12 mj per beam. Referring to FIG. 46, tissue was treated with red organic dye, and FIG. 46 shows the penetration of the dye through the tissue.

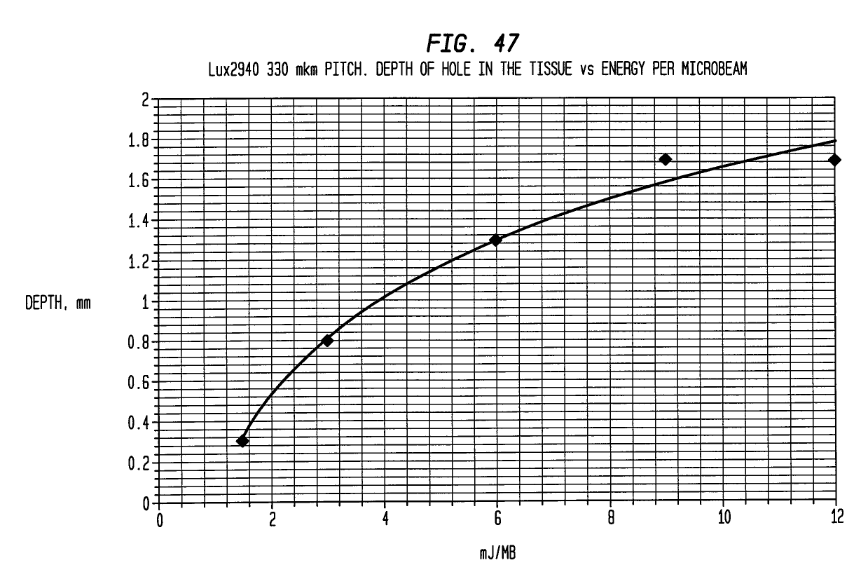

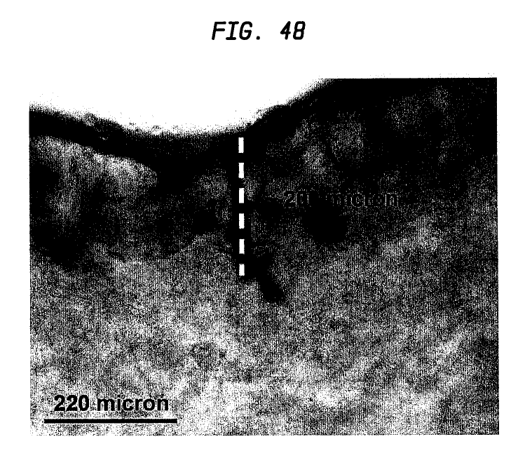

Referring to FIG. 47, the relationship between the depth of micro-holes and the energy used per beam at a wavelength of 2940 nm is shown. Generally, the depth of the micro-holes increases proportionally to the increasing of energy per beam. At the highest energy, the micro-holes traversed from the epidermis and through the hypodermis.

3. Experiment 3—Treatment of Tissue Using 2940 nm and a Pitch of 220 μm

In this experiment, tissue from a Yucatan black pig in vitro was treated with a device similar to device 500 having beam spaced by 220 micrometers, and that irradiated the tissue at a wavelength of 2940 nm. The skin was defrosted prior to testing and warmed to room temperature. The skin was marked with a marking pen and treated with the EMR. The applied energy was verified after every shot of EMR. The glass window of the tip of the applicator was cleaned after each treatment. The energy readings varied by less than 5%.

The skin was then stretched and pinned down on a flat surface. A drop of black tattoo ink was placed on the treated area and massaged into the micro-holes. (In another test, red organic molecules in water (Eosin) were applied to the micro-holes in a method similar to the procedure described for tattoo ink.) The skin was released, and a 6 mm biopsy was obtained from the treated area. The biopsy was frozen and manually cut into 100-300 micron sections. The sections were examined with a BH2 light microscope (Olympus) using a CoolPix-8400 photo camera (Nikon). The skin specimen was treated according to tattoo ink particles trapping method inside of micro channels. The treatment parameters are shown in Table F.

TABLE F

| | | Energy per | # of | Samples | Temperature |
|---|---|---|---|---|---|
| # | Screen of tip | MB, mJ | pulse | collected | of skin, ° C. |
| 1 | with lenses | 0.5 | 1 | 2 to 3 | RT |
| 2 | | 1.5 | | samples | |
| 3 | | 3 | | for | |
| 4 | | 6 | | formalin | |

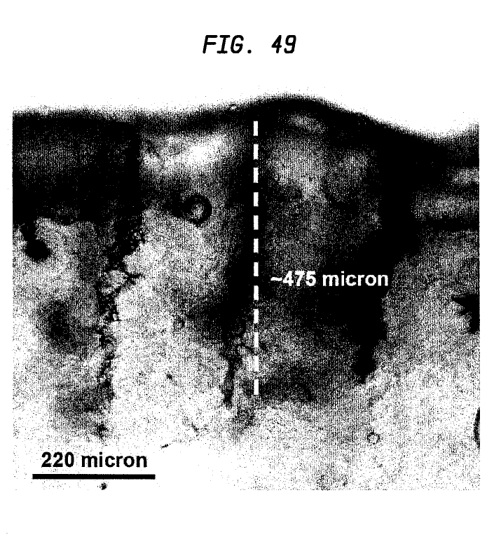

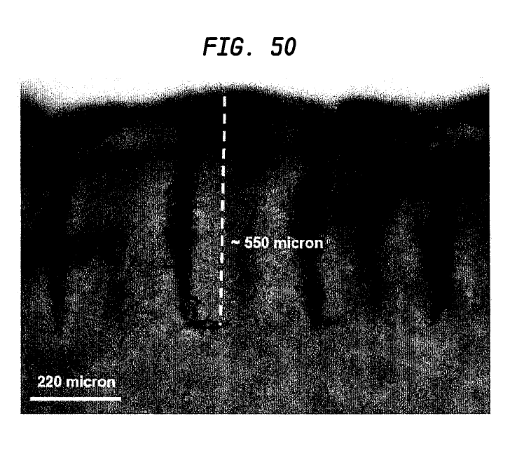

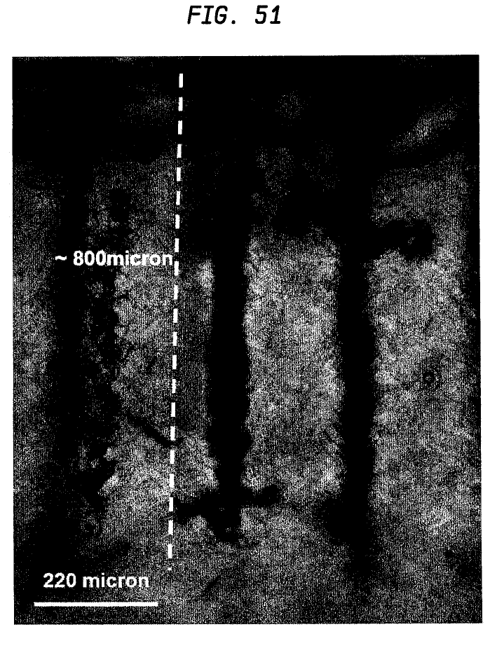

Referring to FIG. 48, a section of pig skin was treated using a device similar to device 500, applying a wavelength of 2940 nm to the tissue in vitro. The treatment parameters were: one pulse of approximately 0.5 mj per beam. The micro-holes traverse through the epidermis and papillary dermis. Referring to FIG. 49, a section of pig skin was treated using a device similar to device 500, applying a wavelength of 2940 nm to the tissue in vitro. The treatment parameters were: one pulse of approximately 1.5 mj per beam. The micro-holes traverse through the epidermis and partially into the dermis. Referring to FIG. 50, a section of pig skin was treated using a device similar to device 500, applying a wavelength of 2940 nm to the tissue in vitro. The treatment parameters were: one pulse of approximately 3.0 mj per beam. The micro-holes traverse through the epidermis and partially into the dermis. Referring to FIG. 51, the skin was treated with one pulse of approximately 6 mj per beam. The micro-holes traverse through the epidermis and larger part of dermis.

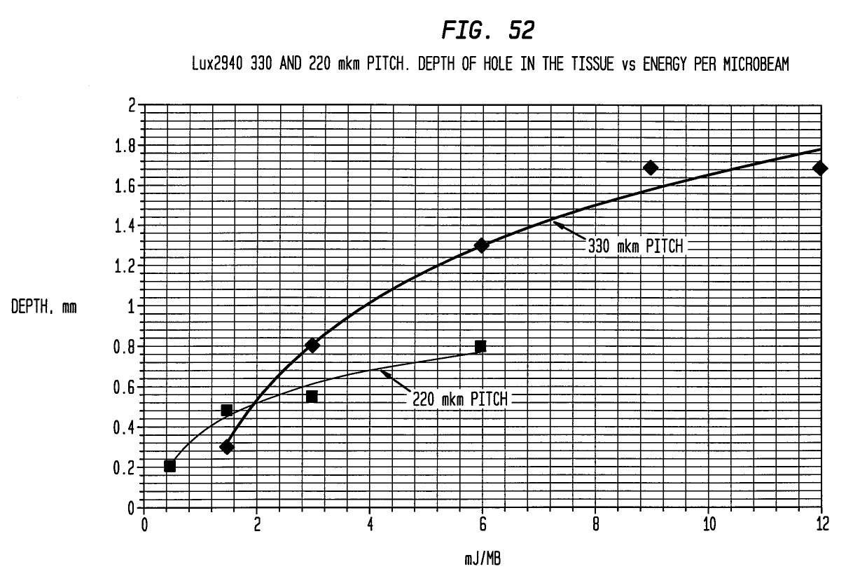

Referring to FIG. 52, the relationship between the depth of micro-holes and the energy used per beam at a wavelength of 2940 nm is shown, where the beams had pitches of 220 and 330 μm respectively. Generally, the depth of the micro-holes increases proportionally to the increasing of energy per beam. At the highest energy, the micro-holes traversed from the epidermis and through the hypodermis. The maximum depth for the 220 μm pitch was approximately 800 μm. The maximum depth for the 330 μm pitch was approximately 1300 μm. These experiments demonstrate, among other things, that the micro-holes can be used for incorporation of drugs and/or other substances, into skin or other tissue in vivo. For example, a drug or other substance having a low absorption rate can be placed in a set of micro-holes for incorporation into the body over a period of time, such as one or more months. Such substances could include, for example, birth control drugs, medications, or a nicotine-containing substance for use by persons in the process of quitting smoking. Many other substances are possible. For example, the substance that can be used depend on the particular medical or cosmetic goal, as well as the setting in which the method is performed and the parameters of the method itself. Non-limiting exemplary substances that can be used with the methods of the present invention, include, for example, topical anesthetics, such as lidocaine, prilocaine; refractive-index-matching compounds, such as glycerol, propylene glycol, glucose solution in water, saline solution for reducing optical scattering (so-called optical clearance) and for better safety and efficacy of subsequent laser/light treatment procedures (for example, hair removal, treatment of vascular and pigmented disorders, skin texture and wrinkle treatment, tattoo removal, cellulite reduction, etc.); chromophores; compounds that promote and facilitate skin rejuvenation and generation of new collagen (for example, alpha hydroxy acids); skin fillers, for example, hyaluronic acid; tattoo or melanin-bleaching compounds; acne-treatment compositions, such as antibiotics, retinoids, benzoyl peroxide; cellulite- and fat-reducing compounds, such as retinol (vitamin A), yohimbine, aminophylline; photosensitizers or photosenistizer-inducing compounds, such as ALA, for subsequent photodynamic therapy (PDT); systemic drugs, nutrients, vitamins, anti-viral drugs, anti-fungal drugs, vaccines; cosmetic dyes, pigments and the like to modify visual appearance of the skin; compounds with high-refractive-index particles as sapphire; compounds with dye such as food dye; compounds with capsulated drugs; compounds with high diffusion coefficient in liquid lipid; topical compositions, such as hydrophilic compounds for facilitating diffusion beyond the stratum corneum, exogenous chromophores, photosensitizers, skin nutrients, and herbal extracts.

By way of example, the tattoo ink that was used in the forgoing experiments do not penetrate the tissue and provide a profile of the resulting channel when imaged. On the other hand, the organic ink molecules (Eosin B) do penetrate through the tissue, and no channel profiles were seen. Thus, given that the molecular weight of the Eosin B is more than 600, substances having a molecular weight less than or equal to 600 likely will penetrate tissue. Thus, the channels can be used to deliver drugs and other substances, preferably having an atomic weight of approximately 600 or less. Further, as a general guide, particles having a diameter of approximately 0.05 μm to 100 μm will likely remain in a micro-hole and not diffuse, be absorbed, or otherwise be incorporated into the tissue. Particles having a diameter of less than approximately 0.05 μm, will likely diffuse, be absorbed or otherwise be incorporated into the tissue.

However, one skilled in the art will appreciate that many other factors will affect whether and to what degree a substance will penetrate into tissue. Thus, in other embodiments, substances having a molecular weight greater than 600 may be used. Similarly, some substances having molecular weights less than 600 may not effectively penetrate into the tissue from the micro-holes due to other factors such as the type of tissue, the size of the micro hole, and the chemical structure and nature of the substance. Similarly, particles having a diameter greater than approximately 0.05 μm may diffuse, be absorbed or otherwise be incorporated into the tissue, and particles having a diameter less than approximately 0.05 μm may not diffuse, be absorbed or otherwise be incorporated into the tissue. Thus, many different embodiments are possible.

H. Delivery of Substances For Absorption Into Tissue To Increase Optical Clearance of the Tissue.

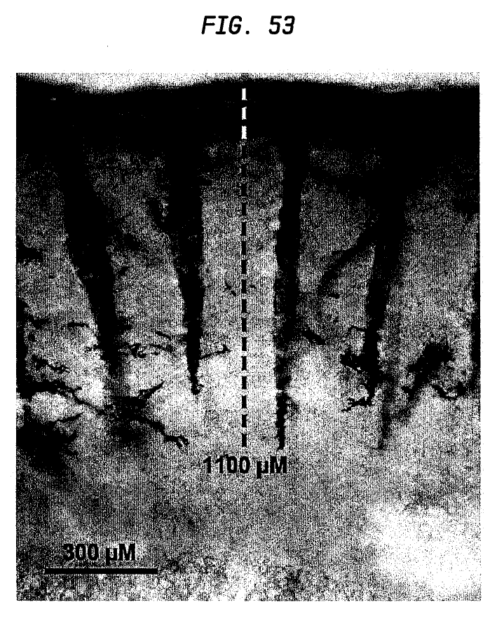

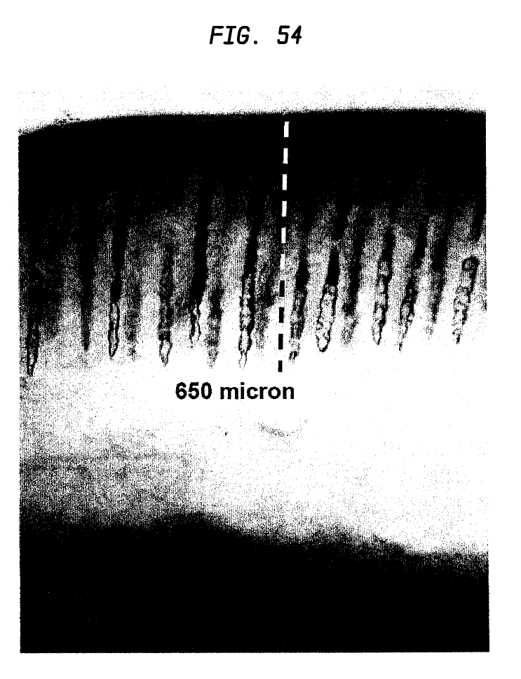

Micro-holes can also be used as channels to inject a clearing compound, such as, for example, glycerol. Referring to FIGS. 46, 53 and 54, the ability to do so was demonstrated in an experiment in which micro-holes were formed using a device similar to device 500 as shown in FIGS. 7-9, and using a wavelength of 2940 nm. Following creation of the micro-channels, glycerol was introduced into the tissue through the micro-holes. Subsequently, ink pigments were encapsulated into the micro-holes using a device that irradiated the tissue using EMR at a wavelength of 1540 nm. The treatment parameters were 2 mj per beam, using 1 pulse. The refraction coefficient of glycerol (˜1.4) is close to refraction coefficient of the dermis of the skin. Therefore, penetration of glycerol into the skin resulted in optical clearance of tissue, and increased transparency.

As shown in FIG. 61, in a second trial, the tissue exhibited diffusion of Eosin Y molecules through the tissue following treatment at 2940 nm. The treatment parameters were also 2 mj per beam using a single pulse. The substance applied was a 2.0% solution of Eosin Y in water. Optical clearance of the tissue was achieved by glycerol/ink pigment mixture. The suspension of ink and glycerol penetrated into the micro-holes. The experiment demonstrated good penetration of the ink pigments, and further demonstrated that the substances can be introduced into the tissue that increase clearance of the tissue. Specifically, the tissue cleared such that multiple rows of micro-holes are visible through the tissue.

The ability to introduce substance that can then diffuse into the tissue to alter the translucence, transparency, and/or opacity of the tissue has many practical applications. For example, such clearing substances can be used to increase the transparency of the skin to provide increased contrast between the tissue and a chromophore prior to irradiating the chromophore with EMR or other energy. This will decrease the amount of energy absorbed by the tissue surrounding the chromophore, increasing the relative selectivity of the chromophore, decreasing the energy required for, e.g., selective thermolysis, reducing or eliminating the need for cooling, and reducing or eliminating pain. Similarly, increasing the transparency of the tissue allows for improved imaging of structures within the tissue, and may allow imaging of some tissues that would otherwise be too opaque to be viewed.

I. The Use of Micro-Holes to Treat Nail Fungus

In one embodiment, using device 500 as shown in FIGS. 7-9, extremely small holes can be created in nail tissue to treat diseases of the nail such as (for instance onychomycosis). Currently such diseases are treated by surgically removing infected nail tissue followed by treatment with antibiotics and other medicines for several weeks. This method is highly effective but complicated, because it involves surgical procedures and the many complications and inconveniences associated with such procedures. Other treatments involve directly applying medicines or other substances to the infected nail. Typically, these treatments are not highly effective because the penetration of medicine and other substances through the nails is low, likely due to the low permeability of nail tissue.

However, EMR-treated islets can be created to treat diseases of the nails, such as onychomycosis and other infectious diseases at the human nails and their surrounding anatomical sites.

The tissue can be treated directly with EMR. Additionally, photodynamic treatment of the tissue by direct photo activation of endogenous photosensitizers can be used by applying one or more wavelengths of light to the photosensitizer.

Another mechanism for the treatment is to enhance penetration of drugs, other substances or other exogenous photosensitizers through the infected nails. One such mechanism is the use of EMR to create an array of traverse micro-holes in a nail. To create an array of micro-holes, several mechanisms may be used, including, without limitation, single or multiple wavelength light, microwave or ultrasound devices. Dimensions and orientations of the micro-holes could be controlled to suit the application. For example, diameters of holes could be 50-75 micron or greater. Similarly, the micro-holes may be perpendicular to the nail surface or at an angle, depending on the treatment requirement.

The depth of the micro-holes also may be controlled. Depths are dependant on the treatment settings, and the depth can be controlled, for example, by applying one or more pulses of EMR, each successively deepening or enlarging the hole. The treatment method may involve sequential treatment with photosensitizers, chromophores, medicines, or washing techniques in any possible order.

Several exemplary approaches for using micro-holes to treat diseases of the nails have been tested. (Other approaches, which have not been tested, are possible, however.) These approaches are: (1) application of an exogenous chromophore to the micro-holes that is activated with EMR following application; (2) the application of drugs or other substances to the micro-holes that is not activated with EMR following application; and (3) washing the affected tissue from underneath the nail using an antiseptic solution.

1. Application of an Exogenous Chromophore

In this embodiment, a suspension or some other formulation of desirable exogenous chromophores (photosensitizers) are applied to the openings of the micro-holes. The chromophores penetrate the nail through the holes, for example by simple diffusion or by employing other approaches, such as vesicle/particle transporters, by physical, chemical or electrical manipulations (for example, electroporation, iontoporation, sonophoresis, magnetophoresis, photomechanical waves, niosomes, transfersomes etc.).

When the chromophore that is applied reaches the targeted areas (such as areas infected with fungus) different wavelengths of light sources can be used for the photodynamic therapy. The wavelength(s) used for the photodynamic therapy will depend on various factors such as the absorption properties of the active compound. Several applications of the active compound and several treatments with EMR may be required. In some embodiments, different EMR sources and different chromophores (photosensitizers) can be used.

2. Application of Drugs and/or Other Substances

In this embodiment, a drug and/or other substance is applied in a manner that is similar to that of Approach One. However, the drug or other substance that is applied is not photoactivated following application. Micro-holes with desirable properties are created. Drugs and/or other substances, such as a topical cream, solution, suspension etc., are applied to the openings of micro-holes in the surface of the nail. The substances applied penetrate the nail by an appropriate method such as vesicle/particle transportation, or by other physical, chemical or electrical methods. In some cases, natural diffusion of the active ingredients of the medicine through the hole may be the most efficient delivery mechanism. Several applications of the active compound and several treatments with EMR may be required in some treatments. The combination of two or more biologically active ingredients can also be used in appropriate circumstances.

3. Washing of an Infected Area

In this embodiment, parasites are washed out from the affected space under the nail. A washing antiseptic solution can be pumped under the nail through the micro-holes by applying of pressure. In some embodiments, the solution can be extracted by creation of a vacuum. One or more multi-cycle pump in/pump out steps could be used to wash out of parasites from the treated area at the desirable level. The removal of parasites at the desirable level may also be achieved by a mechanical wash or with a mechanical removal of the infected matter, which may be followed by, or accomplished in parallel with, the application of a disinfectant in the treated area.

Wash out and disinfection steps could be accomplished with one solution as a one step treatment or sequentially with two solutions. A one step treatment solution could contain antiseptic compound(s) and compound(s) which will enhance detachment of parasites from the treatment area. The entire affected area could be treated as one target region or it could be divided as an independent segments that are treated at different times or using different regimens. The approach could employ one or more treatment cycles or applications.

4. Exemplary Experiments

Several in vitro and in vivo experiments were performed using a device providing an array of beams at a wavelength of 2940 nm. The device was essentially the same as the embodiment described in conjunction with device 500 of 7-9. For each experiment, direct and indirect evidence of the formation of micro-holes was obtained. Micro-holes were created in wet and dry paper, in vivo humans and ex vivo pig skin and in vivo and in vitro human nails. The creation of holes in vitro was verified by observation of the treated items under a microscope. The creation of holes in vivo was verified by observation and by applying a solution of 10% ammonia on the treated spot (on both skin and nail). The experiments demonstrated that EMR can be employed to successfully ablate tissue and create traverse holes in the tissue, including human nails and skin.

a. Experiment 1—Treatment of dry paper

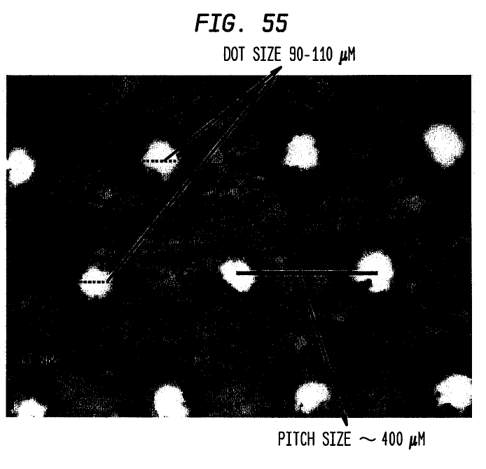

A sheet of paper was treated with EMR at a wavelength of 2940 nm. The treatment parameters were 8-10 mj per beam and a pulse width of 200 microseconds. As is shown in FIG. 55, micro-holes were created having a diameter of 90-110 micrometers and a pitch of 400 micrometers.

b. Experiment 2—Treatment of Wet Paper

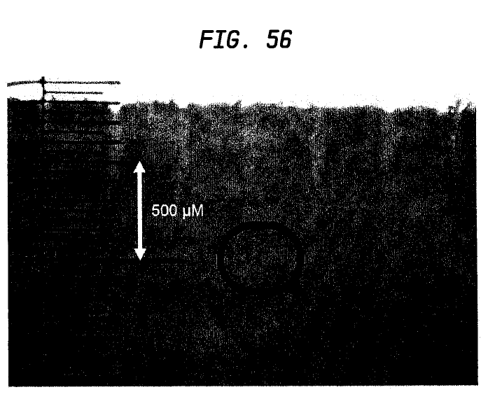

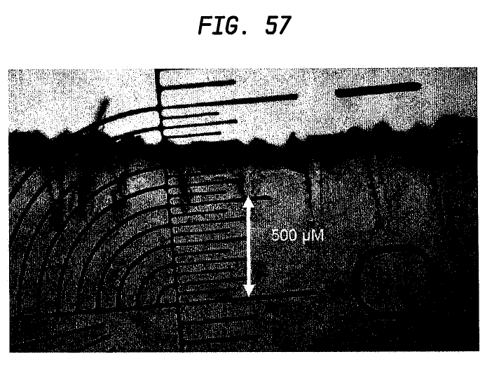

A sheet of paper was wetted and trapped between two glass slides. The slides were oriented parallel to beam trajectory in the same plane. The distance between the paper and device was 1-3 mm. The paper was irradiated with EMR having a wavelength of 2940 nm, at 18-20 mj per beam and a pulse width of 200 microseconds. As it is shown in FIG. 56, the approximate depth of the resulting columns/islets was 350-400 micrometers, while the diameters of the resulting micro-holes were approximately 50-70 micrometers.

c. Experiment 3—Treatment of Slice of Ex Vivo Pig Skin

A thin slice of fresh pig skin was trapped between two glass slides and treated similarly to the wet paper described in Experiment 2, using the same treatment parameters. As it is shown in FIG. 57, the depth of the resulting micro-holes was approximately 350-400 micrometers and the diameter of the micro-holes was approximately 50-75 micrometers.

d. Experiment 4—Traverse Micro-Holes in the Human Nail In Vivo.

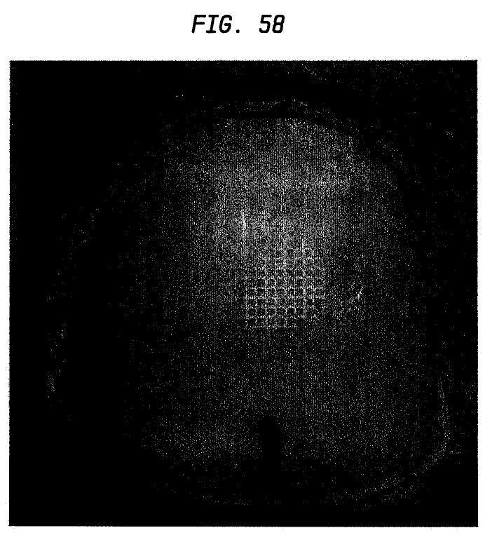

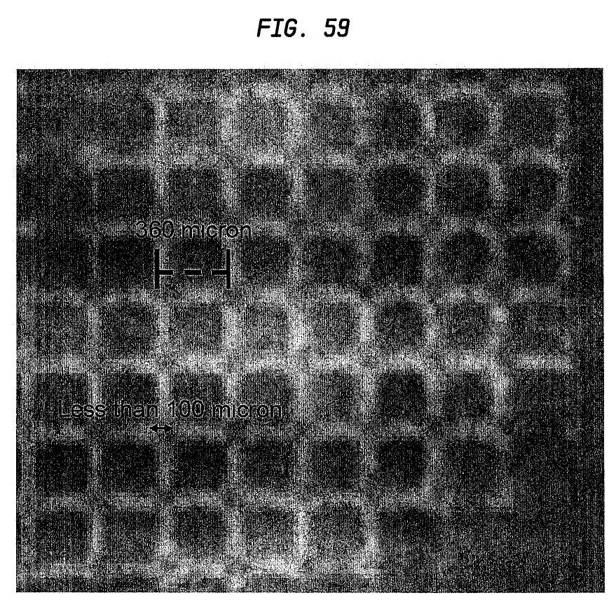

EMR-treated islets were created generally perpendicular to the surface of a human finger nail. The parameters employed in this experiment were the same as those described in Experiments 2 and 3. However, in this case, the laser was fired twice, while it was fired once in Experiments 2 and 3. As a result of the treatment, the subject had a tingling sensation after the second firing but did not experience pain from the treatment. A burning sensation was felt after applying a 10% ammonia solution, which was very similar to the sensation experienced when ammonia contacts broken skin. Referring to FIG. 58, the nail is shown immediately following treatment.

Referring also to FIG. 59, which is a close up view of the same nail, the diameter of the resulting holes is less than approximately 100 micrometers, while the pitch is approximately 360 micrometers. The depth of the holes was difficult to measure. However, because the 10% ammonia was felt through the nail, it is assumed that the depth of the holes was deeper than the cornfield layer of nail.

e. Experiment 5—Traverse Hole in the Human Nail In Vitro.

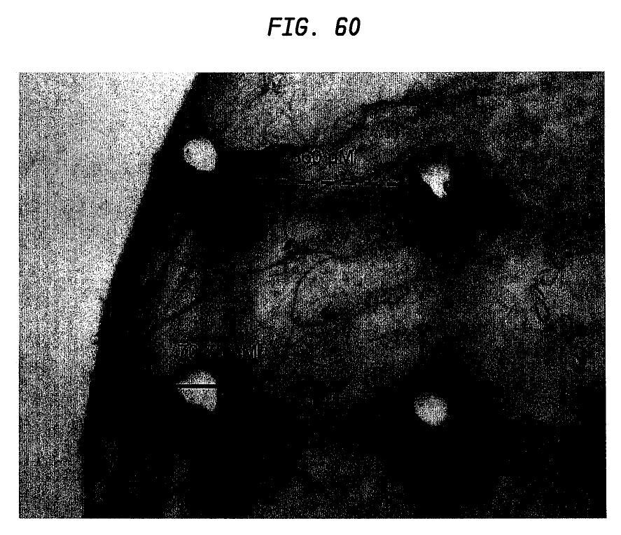

Referring to FIG. 60, a portion of a nail was removed from a subject's thumb and incubated in PBS for approximately 5 minutes. The nail was flattened and located perpendicular to the EMR beam. The nail was greater than approximately 0.4 mm thick. The treatment parameters used were: EMR having a wavelength of 2940; 6-18 mj per beam; and two pulses of 200 microseconds each. The resulting micro-holes had a pitch of 360 micrometers. The micro-holes had diameters of less than approximately 100 micrometers.

The results indicate that EMR at a wavelength of 2940 nm can be used successfully to create micro-holes that extend through human nail tissue and therefore could be used to create channels for the delivery of different pharmaceutical compounds through the human nails.

J. Additional Applications

EMR-treated islets can be used in a variety of applications in a variety of different organs and tissues. For example, EMR treatments can be applied to tissues including, but not limited to, skin, mucosal tissues (e.g., oral mucosa, gastrointestinal mucosa), ophthalmic tissues (e.g., conjunctiva, cornea, retina), and glandular tissues (e.g., lachrymal, prostate glands). As a general matter, the methods can be used to treat conditions including, but not limited to, lesions (e.g., sores, ulcers), acne, rosacea, undesired hair, undesired blood vessels, hyperplastic growths (e.g., tumors, polyps, benign prostatic hyperplasia), hypertrophic growths (e.g., benign prostatic hypertrophy), neovascularization (e.g., tumor-associated angiogenesis), arterial or venous malformations (e.g., hemangiomas, nevus flammeus), and undesired pigmentation (e.g., pigmented birthmarks, tattoos), sebaceous glands, disorders of sebaceous glands, sweat glands (e.g., for permanent reduction of perspiration).

In another embodiment, skin oils, especially on the face, can be reduced by killing or reducing the activity of sebaceous glands. More effective delivery of oil secretion suppressors into skin can also be achieved to control oil levels on the skin surface and reduce oil-induced skin surface brightness (reflectance).

The lattices can be used post-treatment to, for example, facilitate the application and/or absorption of medication to the treated tissue to aid the healing process. Various types of medication can be applied, including topical substances intended to have an immediate effect or capsulated drugs intended to be released slowly. An example of the latter is Vitamin A, which can be applied to be released over and extended period of time (e.g., one month) to further enhance the healing process. Additionally, combinations of medication can be applied. Similarly, antibiotics can be applied to prevent infection, or a film can be applied across the surface of the tissue to prevent infection, such as a polymeric film released or applied across the surface of the tissue following treatment.

### EQUIVALENTS

While only certain embodiments have been described, it will be understood by those skilled in the art that various changes in form and details may be made therein without departing from the spirit and scope as defined by the appended claims. Those skilled in the art will recognize, or be able to ascertain using no more than routine experimentation, many equivalents to the specific embodiments described specifically herein. Such equivalents are intended to be encompassed in the scope of the appended claims.

### References and Definitions

The patent, scientific and medical publications referred to herein establish knowledge that was available to those of ordinary skill in the art. The entire disclosures of the issued U.S. patents, published and pending patent applications, and other references cited herein are hereby incorporated by reference.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the claimed subject matter, the following definitions are provided for certain terms which are used in the specification and appended claims.

### Numerical Ranges

As used herein, the recitation of a numerical range for a variable is intended to convey that the embodiments may be practiced using any of the values within that range, including the bounds of the range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous. Finally, the variable can take multiple values in the range, including any sub-range of values within the cited range.

Or. As used herein, unless specifically indicated otherwise, the word “or” is used in the inclusive sense of “and/or” and not the exclusive sense of “either/or.”

As used herein, EMR includes the range of wavelengths approximately between 200 nm and 10 mm. Optical radiation, i.e., EMR in the spectrum having wavelengths in the range between approximately 200 nm and 100 μm, is preferably employed in some of the embodiments described above, but, also as discussed above, many other wavelengths of energy can be used alone or in combination. Also as discussed, wavelengths in the higher ranges of approximately 2500-3100 nm may be preferable for creating micro-holes using ablative techniques. The term “narrow-band” refers to the electromagnetic radiation spectrum, having a single peak or multiple peaks with FWHM (full width at half maximum) of each peak typically not exceeding 10% of the central wavelength of the respective peak. The actual spectrum may also include broad-band components, either providing additional treatment benefits or having no effect on treatment. Additionally, the term optical (when used in a term other than term “optical radiation”) applies to the entire EMR spectrum. For example, as used herein, the term “optical path” is a path suitable for EMR radiation other than “optical radiation.”

It should be noted, however, that other energy may be used to for treatment islets in similar fashion. For example, sources such as ultrasound, photo-acoustic and other sources of energy may also be used to form treatment islets. Thus, although the embodiments described herein are described with regard to the use of EMR to form the islets, other forms of energy to form the islets are within the scope of the invention and the claims.

We claim:

1. A method of trans-dermal delivery of substances comprising:
ablating a portion of the tissue with electromagnetic radiation to form a plurality of channels extending to a depth below a surface of the tissue;
delivering a substance into one or more of the channels; and
sealing said one or more channels, so as to embed the substance within the channels.

2. The method of claim 1, wherein the step of delivering a substance into the one or more channels further comprises using a delivery technique selected from the group consisting of diffusion, vesicle/particle transporters, physical mechanisms, chemical mechanisms, electrical mechanisms, electroporation, iontoporation, sonophoresis, magnetophoresis, photomechanical waves, niosomes, and transfersomes.

3. The method of claim 1, wherein the step of sealing said one or more channels further comprises using a technique selected from the group consisting of applying a healing substance to said portion of tissue and covering said portion of tissue.

4. The method of claim 3, wherein said covering said portion of tissue further comprises at least one of applying tape or applying sutures to said portion.

5. The method of claim 1, wherein said substance is a drug.

6. The method of claim 1, wherein the channels extend to a depth in a range of approximately 5 micrometers to 5 millimeters.

7. The method of claim 1, wherein the tissue is skin tissue and wherein the channels extend from the surface of the skin tissue to the epidermis.

8. The method of claim 1, wherein the tissue is skin tissue and wherein the channels extend from the surface of the skin tissue to the dermis.

9. The method of claim 1, wherein the tissue is skin tissue and wherein the channels extend from the surface of the skin tissue to below the dermis.

10. The method of claim 1, wherein each of the channels has a width in a range of approximately 1 micrometer to 1 millimeters.

11. The method of claim 1, wherein each of the channels has a width of approximately 50 micrometers to 100 micrometers.

12. The method of claim 1, wherein the fill-factor of the channels in a cross-sectional area is between approximately 1 and 90 percent.

13. The method of claim 1, wherein the fill-factor of the channels in a cross-sectional area in a plane extending through the channels is between approximately 25 and 40 percent.

14. The method of claim 1, wherein the center-to-center distance between adjacent channels is between approximately 10 micrometers and 5 millimeters.

15. The method of claim 1, wherein the tissue is ablated with electromagnetic radiation having one or more wavelengths of between approximately 190 to 350 nanometers.

16. The method of claim 1, wherein the tissue is ablated with electromagnetic radiation having one or more wavelengths of between approximately 1.3 and 12 micrometers.

17. The method of claim 1, further comprising ablating a second portion of the tissue with electromagnetic radiation to form at least one additional channel extending to a depth from a surface of the tissue, wherein the at least one additional channel has dimensions different than those of the plurality of channels.

18. The method of claim 1, wherein the plurality of channels is a first plurality and the method further comprising ablating a second portion of the tissue with electromagnetic radiation to form a second plurality of channels extending to a depth from a surface of the tissue, wherein the second plurality of channels have dimensions different than those of the first plurality of channels.
wherein the plurality of channels is a first plurality, the method further comprising creating a second plurality of channels.

19. The method of claim 1, wherein the plurality of channels includes at least first and second sets of channels, the channels of the first set having dimensions different than the channels of the second set.

20. The method of claim 19, wherein the channels of the first set extend to a first depth and the channels of the second set extend to a second depth that is deeper than the first depth.
